SEQUENCE LISTING (141 sequences)

SEQ ID NO: 1 - DNA, 8 bp, Artificial Sequence
  Description of Artificial Sequence Synthetic oligonucleotide
tataaaag

SEQ ID NO: 2 - DNA, 76 bp, Artificial Sequence
  Description of Artificial Sequence Synthetic oligonucleotide
caacaaaatgtcgtaacaagggcggtaggcgtgtacggtgggaggtctatataagcagagctcgtttagtgaaccg

SEQ ID NO: 3 - DNA, 203 bp, Artificial Sequence
  Description of Artificial Sequence Synthetic polynucleotide
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctt

SEQ ID NO: 4 - DNA, 63 bp, Artificial Sequence
  Description of Artificial Sequence Synthetic oligonucleotide
ttcgcatattaaggtgacgcgtgtggcctcgaacaccgagcgaccctgcagcgacccgcttaa

SEQ ID NO: 5 - DNA, 41 bp, Artificial Sequence
  Description of Artificial Sequence Synthetic oligonucleotide
ggggggctataaaagggggtgggggcgttcgtcctcactct

SEQ ID NO: 6 - DNA, 178 bp, Artificial Sequence
  Description of Artificial Sequence Synthetic polynucleotide
ctgacaaattcagtataaaagcttggggctggggccgagcactggggactttgagggtggccaggccagcgtaggaggccagcgtaggatcctgctgggagcggggaactgagggaagcgacgccgagaaagcaggcgtaccacggagggagagaaaagctccggaagcccagcagcg

SEQ ID NO: 7 - DNA, 47 bp, Artificial Sequence
  Description of Artificial Sequence Synthetic oligonucleotide
agacactagagggtatataatggaagctcgacttccagcttggcaat

SEQ ID NO: 8 - DNA, 44 bp, Artificial Sequence
  Description of Artificial Sequence Synthetic oligonucleotide
tctagagggtttataatgggggccactagtctactactcagaaa

SEQ ID NO: 9 - DNA, 41 bp, Artificial Sequence
  Description of Artificial Sequence Synthetic oligonucleotide
tctttcttttcggttgtcaaaatttcgagcggagcggtcgc

SEQ ID NO: 10 - DNA, 44 bp, Artificial Sequence
  Description of Artificial Sequence Synthetic oligonucleotide
tctagagggtagataatgggggccactagtctactacgagaaag

SEQ ID NO: 11 - DNA, 44 bp, Artificial Sequence
  Description of Artificial Sequence Synthetic oligonucleotide
tctagagggtatataataataaccactagtctactaccagaaag

SEQ ID NO: 12 - DNA, 25 bp, Artificial Sequence
  Description of Artificial Sequence Synthetic oligonucleotide
tctagagggtatataatgggggcca

SEQ ID NO: 13 - DNA, 44 bp, Artificial Sequence
  Description of Artificial Sequence Synthetic oligonucleotide
tctagagggtataaaaggcgggccactagtctactatgtggaag

SEQ ID NO: 14 - DNA, 44 bp, Artificial Sequence
  Description of Artificial Sequence Synthetic oligonucleotide
tctagagggtatataatgggggccactagtctactacgggaaag

SEQ ID NO: 15 - DNA, 45 bp, Artificial Sequence
  Description of Artificial Sequence Synthetic oligonucleotide
tctagagggtatataatgggggccactagtctaactatcagtcag

SEQ ID NO: 16 - DNA, 44 bp, Artificial Sequence
  Description of Artificial Sequence Synthetic oligonucleotide
tctagagggtatataatgggggccactagtctactatcagtcag

SEQ ID NO: 17 - DNA, 44 bp, Artificial Sequence
  Description of Artificial Sequence Synthetic oligonucleotide
tctagagggtataaaaggcgggccactagtctactaccagaaag

SEQ ID NO: 18 - DNA, 44 bp, Artificial Sequence
  Description of Artificial Sequence Synthetic oligonucleotide
tctagagggtatataatgggggccaaagactagtctactaccag

SEQ ID NO: 19 - DNA, 44 bp, Artificial Sequence
  Description of Artificial Sequence Synthetic oligonucleotide
ctagagggtatataatgggggccactagtctactccgtcagatc

SEQ ID NO: 20 - DNA, 44 bp, Artificial Sequence
  Description of Artificial Sequence Synthetic oligonucleotide
tctagagggtataaaaggcgggccactagtctactatcagtcag

SEQ ID NO: 21 - DNA, 44 bp, Artificial Sequence
  Description of Artificial Sequence Synthetic oligonucleotide
tctagagggtatataatgggggccactagtctactaccagaaag

SEQ ID NO: 22 - DNA, 44 bp, Artificial Sequence
  Description of Artificial Sequence Synthetic oligonucleotide
tctagagggcttaaaatgggggccactagtctactaccagaaag

SEQ ID NO: 23 - DNA, 59 bp, Artificial Sequence
  Description of Artificial Sequence Synthetic oligonucleotide
gtaggcgtgtacggtgggaggtctatataagcagagctcgtttagtgaaccgtcagatc

SEQ ID NO: 24 - DNA, 59 bp, Artificial Sequence
  Description of Artificial Sequence Synthetic oligonucleotide
actagagggtatataatggaagctcgacttccagcttggcaatccggtactgttatggc

SEQ ID NO: 25 - DNA, 59 bp, Artificial Sequence
  Description of Artificial Sequence Synthetic oligonucleotide
atgctctagagggtatataatgggggccactagtctactaccagaaagcttggtaccga

SEQ ID NO: 26 - DNA, 108 bp, Artificial Sequence
  Description of Artificial Sequence Synthetic polynucleotide
accgacggacggggcagcgggtacccgctggggcagtgccgcagcggcaggaatacggagggcagcgggtacccgctggggcagtgccgcagcggcaggaatacggag

SEQ ID NO: 27 - DNA, 108 bp, Artificial Sequence
  Description of Artificial Sequence Synthetic polynucleotide
caccagggaaatgagcgtgcgggcagaaggcggaagcaaccatggttacgatatgtatttgttagggcagaaggcggaagcaaccatggttacgatatgtatttgtta

SEQ ID NO: 28 - DNA, 110 bp, Artificial Sequence
  Description of Artificial Sequence Synthetic polynucleotide
agctggatcaaataacaagctggatcaaataacacgctctgaaatagcgctctgaaatagtggggcgggtatggggcgggtatataaatcaataagtataaatcaataag

SEQ ID NO: 29 - DNA, 110 bp, Artificial Sequence
  Description of Artificial Sequence Synthetic polynucleotide
tcttgaagcactccctggaagaggtgggggacaaaatgggtgattgggggacaagttcgggcactgtgggggacaagttcgggcactgtgggggacaagttcgggcactg

SEQ ID NO: 30 - DNA, 110 bp, Artificial Sequence
  Description of Artificial Sequence Synthetic polynucleotide
aactggggcaaaggtcaaactggggcaaaggtcaccctttgatgtggccctttgatgtggaggggcggggcaggggcggggcaaaaaatcgataataaaaaatcgataat

SEQ ID NO: 31 - DNA, 101 bp, Artificial Sequence
  Description of Artificial Sequence Synthetic polynucleotide
gggagatgggggagatgggggtgttaaagggtgttaaatgggggacatgggggacactgagccggtaccctgttgcctgtttcccccgcgctctgaaatag

SEQ ID NO: 32 - DNA, 110 bp, Artificial Sequence
  Description of Artificial Sequence Synthetic polynucleotide
cttggctccctgccaagcttggctccctgccaagttggcacggtgccaattggcacggtgccaaccctttgatgtggccctttgatgtggaggtgtgaaaaggtgtgaaa

SEQ ID NO: 33 - DNA, 108 bp, Artificial Sequence
  Description of Artificial Sequence Synthetic polynucleotide
aaaagaggaagtgaaagtaagagcaggaagtgagcgttgccatggcaacgagggggatcgatgggagcaggaagtgagcgttgccatggcaacgagggggatcgatgg

SEQ ID NO: 34 - DNA, 108 bp, Artificial Sequence
  Description of Artificial Sequence Synthetic polynucleotide
tgggggacatgggggacatgagtgatccgtgagtgatccgcgctctgaaatagcgctctgaaatagtgagtgatccgcgctctgaaatagtgggggacatgggggaca

SEQ ID NO: 35 - DNA, 110 bp, Artificial Sequence
  Description of Artificial Sequence Synthetic polynucleotide
gttgcctgtttcccccggttgcctgtttcccccgctgagccggtaccctctgagccggtaccctcgctctgaaatagcgctctgaaataggggtgttaaagggtgttaaa

SEQ ID NO: 36 - DNA, 110 bp, Artificial Sequence
  Description of Artificial Sequence Synthetic polynucleotide
tcagggaaggcttcctggaggaggtcaaggtcaaattaggtcactcaaggtcaaactaggtcagggtcaaggtcaaactaggtcagggtcaaggtcaaactaggtcaggg

SEQ ID NO: 37 - DNA, 104 bp, Artificial Sequence
  Description of Artificial Sequence Synthetic polynucleotide
tacaagctttgtcaccggtgttgcctgtttcccccggcaaccccctcctgggggacacgctctgaaataggcaaccccctcctgggggacacgctctgaaatag

SEQ ID NO: 38 - DNA, 108 bp, Artificial Sequence
  Description of Artificial Sequence Synthetic polynucleotide
gccgacggagggggcagaggagacccgctccggcattgccgcagcgcgtgcaataaggagggcagaggagacccgctccggcattgccgcagcgcgtgcaataaggag

SEQ ID NO: 39 - DNA, 101 bp, Artificial Sequence
  Description of Artificial Sequence Synthetic polynucleotide
aacagatggaacagatggaggtgtgaaaaggtgtgaaatcaaggtcatcaaggtcattggcacggtgccaacttggctccctgccaagccctttgatgtgg

SEQ ID NO: 40 - DNA, 104 bp, Artificial Sequence
  Description of Artificial Sequence Synthetic polynucleotide
agggaagtgggtcagaaggcttggctccctgccaaggcgaccaccgaatcaaggtcaccctttgatgtgggcgaccaccgaatcaaggtcaccctttgatgtgg

SEQ ID NO: 41 - DNA, 108 bp, Artificial Sequence
  Description of Artificial Sequence Synthetic polynucleotide
tcaaggtcatcaaggtcatgattgacaggtgattgacaggccctttgatgtggccctttgatgtggtgattgacaggccctttgatgtggtcaaggtcatcaaggtca

SEQ ID NO: 42 - DNA, 77 bp, Artificial Sequence
  Description of Artificial Sequence Synthetic oligonucleotide
catatgctctagagggtatataatgggggccactagtctactaccagaaagcttggtaccgagctcggatccagcca

SEQ ID NO: 43 - DNA, 84 bp, Artificial Sequence
  Description of Artificial Sequence Synthetic oligonucleotide
gagatccagtttggacactagagggtatataatggaagctcgacttccagcttggcaatccggtactgttatggcccagtccaa

SEQ ID NO: 44 - DNA, 170 bp, Artificial Sequence
  Description of Artificial Sequence Synthetic polynucleotide
agaagaacaaccgtacgccactaacgatcgaagcttgatcaattgaagaataatagtggaccagccggtatccacagtctcaagagagaggacaggccggtatcgactcaagcgacaggacctacttaattgaggtaatattcgttgtcgagtagaattattcctatacc

SEQ ID NO: 45 - DNA, 14 bp, Unknown
  Description of Unknown Transcription factor binding motif sequence
agggggatcgatgg

SEQ ID NO: 46 - DNA, 14 bp, Unknown
  Description of Unknown Transcription factor binding motif sequence
atatgtatttgtta

SEQ ID NO: 47 - DNA, 9 bp, Unknown
  Description of Unknown Transcription factor binding motif sequence
tcaaggtca

SEQ ID NO: 48 - DNA, 9 bp, Unknown
  Description of Unknown Transcription factor binding motif sequence
tgggggaca

SEQ ID NO: 49 - DNA, 14 bp, Unknown
  Description of Unknown Transcription factor binding motif sequence
gagcaggaagtgag

SEQ ID NO: 50 - DNA, 14 bp, Unknown
  Description of Unknown Transcription factor binding motif sequence
gggcagaaggcgga

SEQ ID NO: 51 - DNA, 20 bp, Unknown
  Description of Unknown Transcription factor binding motif sequence
aaaagaggaagtgaaagtaa

SEQ ID NO: 52 - DNA, 20 bp, Unknown
  Description of Unknown Transcription factor binding motif sequence
caccagggaaatgagcgtgc

SEQ ID NO: 53 - DNA, 11 bp, Unknown
  Description of Unknown Transcription factor binding motif sequence
aggggcggggc

SEQ ID NO: 54 - DNA, 11 bp, Unknown
  Description of Unknown Transcription factor binding motif sequence
tggggcgggta

SEQ ID NO: 55 - DNA, 12 bp, Unknown
  Description of Unknown Transcription factor binding motif sequence
ggcagcgggtac

SEQ ID NO: 56 - DNA, 12 bp, Unknown
  Description of Unknown Transcription factor binding motif sequence
ggcagaggagac

SEQ ID NO: 57 - DNA, 12 bp, Unknown
  Description of Unknown Transcription factor binding motif sequence
tgccgcagcggc

SEQ ID NO: 58 - DNA, 12 bp, Unknown
  Description of Unknown Transcription factor binding motif sequence
tgccgcagcgcg

SEQ ID NO: 59 - DNA, 12 bp, Unknown
  Description of Unknown Transcription factor binding motif sequence
ccgctggggcag

SEQ ID NO: 60 - DNA, 12 bp, Unknown
  Description of Unknown Transcription factor binding motif sequence
ccgctccggcat

SEQ ID NO: 61 - DNA, 12 bp, Unknown
  Description of Unknown Transcription factor binding motif sequence
aggaatacggag

SEQ ID NO: 62 - DNA, 12 bp, Unknown
  Description of Unknown Transcription factor binding motif sequence
tgcaataaggag

SEQ ID NO: 63 - DNA, 12 bp, Unknown
  Description of Unknown Transcription factor binding motif sequence
accgacggacgg

SEQ ID NO: 64 - DNA, 12 bp, Unknown
  Description of Unknown Transcription factor binding motif sequence
gccgacggaggg

SEQ ID NO: 65 - DNA, 9 bp, Unknown
  Description of Unknown Transcription factor binding motif sequence
aacagatgg

SEQ ID NO: 66 - DNA, 9 bp, Unknown
  Description of Unknown Transcription factor binding motif sequence
gggagatgg

SEQ ID NO: 67 - DNA, 15 bp, Unknown
  Description of Unknown Transcription factor binding motif sequence
ttggcacggtgccaa

SEQ ID NO: 68 - DNA, 15 bp, Unknown
  Description of Unknown Transcription factor binding motif sequence
ctgagccggtaccct

SEQ ID NO: 69 - DNA, 17 bp, Unknown
  Description of Unknown Transcription factor binding motif sequence
cttggctccctgccaag

SEQ ID NO: 70 - DNA, 17 bp, Unknown
  Description of Unknown Transcription factor binding motif sequence
gttgcctgtttcccccg

SEQ ID NO: 71 - DNA, 19 bp, Unknown
  Description of Unknown Transcription factor binding motif sequence
tacaagctttgtcaccggt

SEQ ID NO: 72 - DNA, 19 bp, Unknown
  Description of Unknown Transcription factor binding motif sequence
ggaaatttgggacagaaga

SEQ ID NO: 73 - DNA, 19 bp, Unknown
  Description of Unknown Transcription factor binding motif sequence
agggaagtgggtcagaagg

SEQ ID NO: 74 - DNA, 19 bp, Unknown
  Description of Unknown Transcription factor binding motif sequence
aagcaagtgggtcagaagg

SEQ ID NO: 75 - DNA, 19 bp, Unknown
  Description of Unknown Transcription factor binding motif sequence
agcaaactgggcctatcga

SEQ ID NO: 76 - DNA, 19 bp, Unknown
  Description of Unknown Transcription factor binding motif sequence
ggctatcttgggcattcgg

SEQ ID NO: 77 - DNA, 14 bp, Unknown
  Description of Unknown Transcription factor binding motif sequence
aaaaaatcgataat

SEQ ID NO: 78 - DNA, 14 bp, Unknown
  Description of Unknown Transcription factor binding motif sequence
tataaatcaataag

SEQ ID NO: 79 - DNA, 11 bp, Unknown
  Description of Unknown Transcription factor binding motif sequence
tgattgacagg

SEQ ID NO: 80 - DNA, 11 bp, Unknown
  Description of Unknown Transcription factor binding motif sequence
tgagtgatccg

SEQ ID NO: 81 - DNA, 17 bp, Unknown
  Description of Unknown Transcription factor binding motif sequence
aactggggcaaaggtca

SEQ ID NO: 82 - DNA, 17 bp, Unknown
  Description of Unknown Transcription factor binding motif sequence
agctggatcaaataaca

SEQ ID NO: 83 - DNA, 16 bp, Unknown
  Description of Unknown Transcription factor binding motif sequence
cgttgccatggcaacg

SEQ ID NO: 84 - DNA, 16 bp, Unknown
  Description of Unknown Transcription factor binding motif sequence
agcaaccatggttacg

SEQ ID NO: 85 - DNA, 13 bp, Unknown
  Description of Unknown Transcription factor binding motif sequence
aactaggtcaggg

SEQ ID NO: 86 - DNA, 13 bp, Unknown
  Description of Unknown Transcription factor binding motif sequence
agttcgggcactg

SEQ ID NO: 87 - DNA, 11 bp, Unknown
  Description of Unknown Transcription factor binding motif sequence
aaatgggtgat

SEQ ID NO: 88 - DNA, 11 bp, Unknown
  Description of Unknown Transcription factor binding motif sequence
aattaggtcac

SEQ ID NO: 89 - DNA, 11 bp, Unknown
  Description of Unknown Transcription factor binding motif sequence
aaataggtcag

SEQ ID NO: 90 - DNA, 11 bp, Unknown
  Description of Unknown Transcription factor binding motif sequence
aaccaggtcaa

SEQ ID NO: 91 - DNA, 11 bp, Unknown
  Description of Unknown Transcription factor binding motif sequence
acttaggtcaa

SEQ ID NO: 92 - DNA, 11 bp, Unknown
  Description of Unknown Transcription factor binding motif sequence
ttgtatgtcat

SEQ ID NO: 93 - DNA, 10 bp, Unknown
  Description of Unknown Transcription factor binding motif sequence
aggtgtgaaa

SEQ ID NO: 94 - DNA, 10 bp, Unknown
  Description of Unknown Transcription factor binding motif sequence
gggtgttaaa

SEQ ID NO: 95 - DNA, 13 bp, Unknown
  Description of Unknown Transcription factor binding motif sequence
ccctttgatgtgg

SEQ ID NO: 96 - DNA, 13 bp, Unknown
  Description of Unknown Transcription factor binding motif sequence
cgctctgaaatag

SEQ ID NO: 97 - DNA, 12 bp, Unknown
  Description of Unknown Transcription factor binding motif sequence
gcgaccaccgaa

SEQ ID NO: 98 - DNA, 12 bp, Unknown
  Description of Unknown Transcription factor binding motif sequence
gcaaccccctcc

SEQ ID NO: 99 - DNA, 24 bp, Unknown
  Description of Unknown Transcription factor binding motif sequence
tcagggaaggcttcctggaggagg

SEQ ID NO: 100 - DNA, 24 bp, Unknown
  Description of Unknown Transcription factor binding motif sequence
tcttgaagcactccctggaagagg

SEQ ID NO: 101 - DNA, 14 bp, Unknown
  Description of Unknown Transcription factor binding motif sequence
atggacacgtcacc

SEQ ID NO: 102 - DNA, 13 bp, Unknown
  Description of Unknown Transcription factor binding motif sequence
tgctttccaggaa

SEQ ID NO: 103 - DNA, 12 bp, Unknown
  Description of Unknown Transcription factor binding motif sequence
cggcacgtgcca

SEQ ID NO: 104 - DNA, 17 bp, Unknown
  Description of Unknown Transcription factor binding motif sequence
cttggttccgttccact

SEQ ID NO: 105 - DNA, 13 bp, Unknown
  Description of Unknown Transcription factor binding motif sequence
tcctttgatgacg

SEQ ID NO: 106 - DNA, 15 bp, Unknown
  Description of Unknown Transcription factor binding motif sequence
aacaattgcattgtt

SEQ ID NO: 107 - DNA, 13 bp, Unknown
  Description of Unknown Transcription factor binding motif sequence
ccctttccaagaa

SEQ ID NO: 108 - DNA, 12 bp, Unknown
  Description of Unknown Transcription factor binding motif sequence
cggcacgtgccg

SEQ ID NO: 109 - DNA, 13 bp, Unknown
  Description of Unknown Transcription factor binding motif sequence
ccctttgatgttg

SEQ ID NO: 110 - DNA, 9 bp, Unknown
  Description of Unknown Transcription factor binding motif sequence
cacagatgg

SEQ ID NO: 111 - DNA, 12 bp, Unknown
  Description of Unknown Transcription factor binding motif sequence
tggcacgtgcca

SEQ ID NO: 112 - DNA, 9 bp, Unknown
  Description of Unknown Transcription factor binding motif sequence
tcaagctcg

SEQ ID NO: 113 - DNA, 170 bp, Artificial Sequence
  Description of Artificial Sequence Synthetic polynucleotide
aaggtagcttccaaccgacggacggggcagcgggtacccgctggggcagtgccgcagcggcaggaatacggagggcagcgggtacccgctggggcagtgccgcagcggcaggaatacggagcagacactagagggtatataatggaagctcgacttccagcttggcaatc

SEQ ID NO: 114 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtttataatgggggccactagtctactactcagaaac

SEQ ID NO: 115 - DNA, 170 bp, Artificial Sequence
  Description of Artificial Sequence Synthetic polynucleotide
aaggtagcttccagtacgcaccgacggacggggcagcgggtacccgctggggcagtgccgcagcggcaggaatacggagggcagcgggtacccgctggggcagtgccgcagcggcaggaatacggagttctttcttttcggttgtcaaaatttcgagcggagcggtcgcc

SEQ ID NO: 116 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtagataatgggggccactagtctactacgagaaagc

SEQ ID NO: 117 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtatataataataaccactagtctactaccagaaagc

SEQ ID NO: 118 - DNA, 170 bp, Artificial Sequence
  Description of Artificial Sequence Synthetic polynucleotide
aaggtagcttccagtacgcctcgttacttcggagtaccgacggacggggcagcgggtacccgctggggcagtgccgcagcggcaggaatacggagggcagcgggtacccgctggggcagtgccgcagcggcaggaatacggagttctagagggtatataatgggggccac

SEQ ID NO: 119 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtataaaaggcgggccactagtctactatgtggaagc

SEQ ID NO: 120 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtatataatgggggccactagtctactacgggaaagc

SEQ ID NO: 121 - DNA, 170 bp, Artificial Sequence
  Description of Artificial Sequence Synthetic polynucleotide
aaggtagcttccagtaccgacggacggggcagcgggtacccgctggggcagtgccgcagcggcaggaatacggagggcagcgggtacccgctggggcagtgccgcagcggcaggaatacggagatctagagggtatataatgggggccactagtctaactatcagtcagc

SEQ ID NO: 122 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtatataatgggggccactagtctactatcagtcagc

SEQ ID NO: 123 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtataaaaggcgggccactagtctactaccagaaagc

SEQ ID NO: 124 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtatataatgggggccaaagactagtctactaccagc

SEQ ID NO: 125 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagtctagagggtatataatgggggccactagtctactccgtcagatcc

SEQ ID NO: 126 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtataaaaggcgggccactagtctactatcagtcagc

SEQ ID NO: 127 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggtatataatgggggccactagtctactaccagaaagc

SEQ ID NO: 128 - DNA, 170 bp, Artificial Sequence
  Description of Artificial Sequence Synthetic polynucleotide
aaggtagcttccagtaaccgacggacggggcagcgggtacccgctggggcagtgccgcagcggcaggaatacggagggcagcgggtacccgctggggcagtgccgcagcggcaggaatacggagttctagagggcttaaaatgggggccactagtctactaccagaaagc

SEQ ID NO: 129 - DNA, 170 bp, Artificial Sequence
  Description of Artificial Sequence Synthetic polynucleotide
aaccgacggacggggcagcgggtacccgctggggcagtgccgcagcggcaggaatacggagggcagcgggtacccgctggggcagtgccgcagcggcaggaatacggagggtaggcgtgtacggtgggaggtctatataagcagagctcgtttagtgaaccgtcagatcc

SEQ ID NO: 130 - DNA, 170 bp, Artificial Sequence
  Description of Artificial Sequence Synthetic polynucleotide
aaccgacggacggggcagcgggtacccgctggggcagtgccgcagcggcaggaatacggagggcagcgggtacccgctggggcagtgccgcagcggcaggaatacggaggactagagggtatataatggaagctcgacttccagcttggcaatccggtactgttatggcc

SEQ ID NO: 131 - DNA, 170 bp, Artificial Sequence
  Description of Artificial Sequence Synthetic polynucleotide
aaccgacggacggggcagcgggtacccgctggggcagtgccgcagcggcaggaatacggagggcagcgggtacccgctggggcagtgccgcagcggcaggaatacggaggatgctctagagggtatataatgggggccactagtctactaccagaaagcttggtaccgac

SEQ ID NO: 132 - DNA, 200 bp, Artificial Sequence
  Description of Artificial Sequence Synthetic polynucleotide
aggaccggatcaactaaggtagcttccagtacgcctcgttacttcggagttacgtatactcacgcgtaagttgccgaataggtgcactatgactggagtgcttagcgcgtgattactgatggacacgtcaccttggcgattctagagggtatataatgggggccactagtctactaccagaaagcttggtaccgagctcg

SEQ ID NO: 133 - DNA, 200 bp, Artificial Sequence
  Description of Artificial Sequence Synthetic polynucleotide
aggaccggatcaactagaagaacaaccgtacgccactaacgatcgaagcttgatcaattgaagaataatagtggaccagccggtatccacagtctcaagaccctttccaagaaggtatctgctttccaggaaggacctactctagagggtatataatgggggccactagtctactaccagaaagcttggtaccgagctcg

SEQ ID NO: 134 - DNA, 200 bp, Artificial Sequence
  Description of Artificial Sequence Synthetic polynucleotide
aggaccggatcaactaaggtagcttccagtacgcctcgttacttcggagttacgtatactcacgcgtaagttgccgaataggtgtggcacgtgccaagtgctcggcacgtgccgactgctcggcacgtgccattggcgattctagagggtatataatgggggccactagtctactaccagaaagcttggtaccgagctcg

SEQ ID NO: 135 - DNA, 200 bp, Artificial Sequence
  Description of Artificial Sequence Synthetic polynucleotide
aggaccggatcaactaaggtagcttccagtacgcctcgagggaagtgggtcagaaggactcacggaaatttgggacagaagatgcactaagcaagtgggtcagaaggcgtgatagggaagtgggtcagaaggttggcgattctagagggtatataatgggggccactagtctactaccagaaagcttggtaccgagctcg

SEQ ID NO: 136 - DNA, 200 bp, Artificial Sequence
  Description of Artificial Sequence Synthetic polynucleotide
aggaccggatcaactaaggtagcttccagtacgcctcgttacttcggagttacgtatactcaccctttgatgtgggaatagtcctttgatgacgggagtgccctttgatgttgtactgcccctttgatgtggttggcgattctagagggtatataatgggggccactagtctactaccagaaagcttggtaccgagctcg

SEQ ID NO: 137 - DNA, 200 bp, Artificial Sequence
  Description of Artificial Sequence Synthetic polynucleotide
aggaccggatcaactaaggtagcttccagtacgcctcgttacttcggagttacgtatactcatcaaggtcatgccgacttggttccgttccactggagtgtcaaggtcatgattacttggttccgttccactttggcgattctagagggtatataatgggggccactagtctactaccagaaagcttggtaccgagctcg

SEQ ID NO: 138 - DNA, 200 bp, Artificial Sequence
  Description of Artificial Sequence Synthetic polynucleotide
aggaccggatcaactaaggtagcttccagtacgcctcgttacttcggagtggaaatttgggacagaagagttgcctcctttgatgacgatgactggaaatttgggacagaagatactgctcctttgatgacgttggcgattctagagggtatataatgggggccactagtctactaccagaaagcttggtaccgagctcg

SEQ ID NO: 139 - DNA, 200 bp, Artificial Sequence
  Description of Artificial Sequence Synthetic polynucleotide
aggaccggatcaactagaagaacaaccgtacgccactaacgatcgaagcttgatcaattgaagaataatagtggaccagccggtatctcaagctcgaagagacacagatggccggtaaacaattgcattgttggacctactctagagggtatataatgggggccactagtctactaccagaaagcttggtaccgagctcg

SEQ ID NO: 140 - DNA, 200 bp, Artificial Sequence
  Description of Artificial Sequence Synthetic polynucleotide
aggaccggatcaactagaagaacaaccgtacgccactaacgatcgaagcttgatcaattgaagaataatagtggaccagccggtatccacagtctcaagagagaggacaggccggtatcgactcaagcgacaggacctacttatctttcttttcggttgtcaaaatttcgagcggagcggtcgccttggtaccgagctcg

SEQ ID NO: 141 - DNA, 170 bp, Artificial Sequence
  Description of Artificial Sequence Synthetic polynucleotide
aaggtagcttccagtacgcctcgttacttcggagttacgtatactcacgcgtaagttgccgaataggtgcactatgactggagtgcttagcgcgtgattactgctggaggattggaattggcgattcttacgcggaaccacgataacgagataacgttaagtcgctagac

(19) United States
(12) Patent Application Publication
(10) Pub. No.: US 2024/0238447 A1
(43) Pub. Date: Jul. 18, 2024

(54) COMPOSITIONS AND METHODS FOR MODULATING PAYLOAD EXPRESSION AT A TRANSCRIPTIONAL LEVEL

(71) Applicant: Shape Therapeutics Inc., Seattle, WA (US)

(72) Inventors: Jacob Michael TOME, Seattle, WA (US); Richard SULLIVAN, Seattle, WA (US)

(21) Appl. No.: 18/561,718

(22) PCT Filed: May 18, 2022
(86) PCT No.: PCT/US22/29888

§ 371 Date: Nov. 16, 2023

Related U.S. Application Data
(60) Provisional application No. 63/190,706, filed on May 19, 2021.

(51) Int. Cl. A61K 48/00; A61K 38/00; C07K 14/47; C12N 15/86

(52) U.S. Cl. CPC A61K 48/0058; C07K 14/4702; C12N 15/86; A61K 38/00; C12N 2750/14143

(57) ABSTRACT

Described herein are compositions comprising polynucleotides encoding for cell state specific transcription of a transgene. A polynucleotide may comprise a promoter that is engineered to promote cell state-specific transcription of a payload (e.g., a transgene). Also described herein are methods of treating, preventing, or ameliorating a disease or condition by delivering a polynucleotide to a cell of a subject and transcribing a transgene in a cell state-specific manner.

### CROSS-REFERENCE

The present application claims the benefit of U.S. Provisional Application No. 63/190,706, entitled “COMPOSITIONS AND METHODS FOR MODULATING PAYLOAD EXPRESSION AT A TRANSCRIPTIONAL LEVEL,” filed on May 19, 2021, which application is herein incorporated by reference in its entirety for all purposes.

### BACKGROUND

A wide variety of diseases and disorders are caused by mutations, deletions, or altered expression of genes. Many of these genes are tightly regulated in healthy individuals such that over-expression or under-expression of the gene may result in detrimental side effects. Additionally, some diseases and disorders are characterized by different cell genotypes of healthy and diseased cells within a subject. While substantial progress is being made toward delivery of transgenes into individuals for treatment of genetic disorders, there remains a need for gene therapies that can regulate transgene expression in a cell-type or cell state dependent manner.

### SUMMARY

In various aspects, the present disclosure provides a recombinant transcription factor binding polynucleotide comprising a sequence having at least 95% sequence identity to SEQ ID NO: 26.

In some aspects, the recombinant transcription factor binding polynucleotide comprises the sequence of SEQ ID NO: 26.

In some aspects, the recombinant transcription factor binding polynucleotide is capable of binding to a transcription factor, optionally, wherein the transcription factor is expressed more highly in a target cell than in a non-target cell. In some aspects, the target cell is a cell expressing a mutant protein, and wherein the non-target cell is a cell expressing a wild type protein. In some aspects, the target cell expresses a mutant MeCP2 protein, and wherein the non-target cell expresses a wild type MeCP2 protein. In some aspects, the recombinant transcription factor binding polynucleotide comprises DNA. In some aspects the recombinant transcription factor binding polynucleotide consists of DNA.

In various aspects, the present disclosure provides a recombinant polynucleotide comprising a promoter and a payload, wherein the promoter comprises: a transcription factor binding polynucleotide capable of binding to a transcription factor, wherein the transcription factor binding polynucleotide comprises a recombinant transcription factor binding polynucleotide as described herein, and a core promoter capable of recruiting a polymerase; wherein the payload comprises a coding sequence.

In some aspects, the promoter comprises: a) a sequence having at least 90% sequence identity to any one of SEQ ID NO: 113-SEQ ID NO: 131; b) a sequence having at least 95% sequence identity to any one of SEQ ID NO: 113-SEQ ID NO: 131; c) a sequence of any one of SEQ ID NO: 113-SEQ ID NO: 131; d) a sequence having at least 90% sequence identity to SEQ ID NO: 115; e) a sequence having at least 95% sequence identity to SEQ ID NO: 115; or f) a sequence of SEQ ID NO: 115. In some aspects, the core promoter comprises a TATA box, an initiator sequence, an RNA polymerase binding sequence, a B recognition element, a CCAAT box, a Pribnow box, a sequence provided in TABLE 4, or combinations thereof.

In some aspects, the coding sequence is capable of being transcribed by the polymerase upon binding of the transcription factor to the transcription factor binding polynucleotide and recruitment of the polymerase to the core promoter; optionally, wherein the polymerase is an RNA polymerase II. In some aspects, the coding sequence encodes a protein. In some aspects, the protein is a neuronal protein; optionally, wherein the protein is associated with a genetic disorder, a neuronal disorder, or both. In some aspects, the protein is MeCP2.

In some aspects, the coding sequence encodes a therapeutic polynucleotide; optionally, wherein the therapeutic polynucleotide is a gRNA or a tRNA. In some aspects, the therapeutic polynucleotide targets a gene associated with a genetic disorder, a neuronal disorder, or both. In some aspects, the therapeutic polynucleotide targets MECP2.

In some aspects, the promoter is engineered to control a transcription level of the payload. In some aspects, the transcriptional level is cell state-specific, cell type-specific, cell genotype-specific, or any combination thereof. In some aspects, a transcriptional level in a target cell is at least 1.3-fold a transcriptional level in a non-target cell.

In various aspects, the present disclosure provides an engineered viral vector comprising a recombinant polynucleotide as described herein in a viral vector; optionally, wherein the viral vector is an adenoviral vector, an adeno-associated viral vector, or a lentivector.

In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68. In some aspects, a viral capsid of the viral vector is from a first viral vector and a viral inverted terminal repeat sequence of the viral vector is from a second viral vector; optionally, wherein the first viral vector, the second viral vector, or both is an adeno-associated viral vector.

In various aspects, the present disclosure provides a pharmaceutical composition comprising a recombinant polynucleotide as described herein or a viral vector as described herein, and a pharmaceutically acceptable carrier.

In various aspects, the present disclosure provides a composition comprising a recombinant polynucleotide as described herein, a viral vector as described herein, or a pharmaceutical composition as described herein for use in a method of treating a disorder in a subject in need thereof, the method comprising administering to the subject the composition, thereby treating the disorder.

In some aspects, a level of transcription of the coding sequence is higher in the target cell than in a non-target cell of the subject. In some aspects, the disorder is a genetic disorder, a neuronal disorder, or both; optionally, wherein the disorder is Rett syndrome.

In various aspects, the present disclosure provides a composition comprising a recombinant polynucleotide as described herein, a viral vector as described herein, or a pharmaceutical composition as described herein for use in a method of expressing a coding sequence in a target cell, the method comprising administering to the subject the composition, thereby expressing the coding sequence in the target cell.

In some aspects, the transcription factor is present at a higher level in the target cell than in the non-target cell; optionally, wherein the transcription factor is more active in the target cell than in the non-target cell. In some aspects, the non-target cell is a healthy cell. In some aspects, the target cell is a neuron. In some aspects, the target cell is a diseased cell; optionally, wherein the diseased cell comprises a genetic mutation associated with the disorder and has a disease phenotype associated with the genetic mutation. In some aspects, the diseased cell comprises a mutation in MECP2 and expresses a mutant MeCP2 protein. In some aspects, a level of transcription of the coding sequence is higher in the target cell than in a non-target cell; optionally, wherein the target cell is a mutant MeCP2 cell, and the non-target cell is a wild type MeCP2 cell.

In some aspects, the method further comprises expressing a protein encoded by the coding sequence in the target cell; optionally, wherein a level of expression of the protein is higher in the target cell than in the non-target cell. In some aspects, the protein is a neuronal protein. In some aspects, the protein is associated with a genetic disorder, a neuronal disorder, or both; optionally, wherein the protein is MeCP2. In some aspects, the method further comprises expressing a therapeutic polynucleotide encoded by the coding sequence in the target cell; optionally, wherein the therapeutic polynucleotide is a gRNA or a tRNA. In some aspects, a level of expression of the therapeutic polynucleotide is higher in the target cell than in the non-target cell. In some aspects, the therapeutic polynucleotide targets a gene associated with a genetic disorder, a neuronal disorder, or both; optionally wherein the therapeutic polynucleotide targets MECP2. In some aspects, the therapeutic polynucleotide targets MECP2. In some aspects, the coding sequence is transcribed upon binding of the transcription factor to the transcription factor binding site and recruitment of the polymerase to the core promoter sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates RNA sequencing (RNA-seq) data showing the fold-change in expression of transcription factors in neurons expressing a wild type MeCP2 protein (“WT McCP2 neuron”) relative to neurons expressing a mutant MeCP2 protein (“mutant MeCP2 neuron”).

FIG. 1B illustrates RNA sequencing data showing the fold-change in expression of transcription factors in neurons relative to hepatocytes (“liver”).

FIG. 1C illustrates RNA sequencing data showing the transcription factor (TF) expression in hepatocytes, in transcripts per kilobase million (TPM), relative to neurons.

FIG. 2A illustrates RNA-sequencing data showing correlation of expression levels of transcription factors between two wild type MeCP2 neuronal cell replicates derived from a Rett patient induced pluripotent stem cell (iPSC) line. Transcription factor expression level (transcripts per kilobase million (TPM)) are shown. Transcription factor expression level for one or more of the 89 candidate transcription factors for being MeCP2 mutant cell specific are shown as darker grey points. The top ten transcription factor candidate expression levels are shown in lighter grey points.

FIG. 2B illustrates RNA-sequencing data showing correlation of expression levels of transcription factors between wild type MeCP2 and mutant MeCP2 neuronal cells derived from a Rett patient iPSC line. Transcription factor expression level (transcripts per kilobase million (TPM)) are shown. Transcription factor expression level for one or more of the 89 candidate transcription factors for being MeCP2 mutant cell specific are shown as darker grey points. The expression levels of the top 10 candidate transcription factors for being MeCP2 mutant cell specific are shown as lighter grey points.

FIG. 2C illustrates RNA-sequencing data showing correlation of expression levels of transcription factors between a wild type MeCP2 neuronal cell derived from a first Rett patient iPSC line and a wild type MeCP2 neuronal cell derived from a second Rett patient iPSC line. Transcription factor expression level (transcripts per kilobase million (TPM)) are shown. Transcription factor expression level for one or more of the 89 candidate transcription factors for being MeCP2 mutant cell specific are shown as darker grey points. The expression levels of the top 10 candidate transcription factors for being MeCP2 mutant cell specific are shown as lighter grey points.

FIG. 2D illustrates RNA-sequencing data showing correlation of enrichment levels of promoters from a library of promoters between wild type MeCP2 and mutant MeCP2 in neuronal cells derived from a third Rett patient iPSC line. Transcription factor expression level (transcripts per kilobase million (TPM)) are shown. Transcription factor expression level for one or more of the 89 candidate transcription factors for being MeCP2 mutant cell specific are shown as darker grey points. The expression levels of the top 10 candidate transcription factors for being MeCP2 mutant cell specific are shown as lighter grey points.

FIG. 3 schematically illustrates examples of promoters comprising an inducible core promoter scaffold (core promoter) and a transcription factor binding sequence (TF Binding Sequence) to be screened for cell state specific transcription.

FIG. 4 schematically illustrates a workflow for engineering and screening promoters with different transcription factor binding sequences for cell state specific expression.

FIG. 5 schematically illustrates examples of engineered promoters with different transcription factor binding sequences to be screened for cell state specific expression.

FIG. 6 schematically illustrates examples of engineered promoters with different transcription factor binding sequences to be screened for cell state specific expression.

FIG. 7 schematically illustrates examples of engineered promoters with different transcription factor binding sequences to be screened for cell state specific expression.

FIG. 8 shows sequences of engineered core promoters of SEQ ID NO: 12, SEQ ID NO: 42, SEQ ID NO: 21, SEQ ID NO: 23, SEQ ID NO: 5, SEQ ID NO: 9, SEQ ID NO: 7, SEQ ID NO: 43, SEQ ID NO: 18, SEQ ID NO: 16, SEQ ID NO: 15, SEQ ID NO: 20, SEQ ID NO: 17, SEQ ID NO: 13, SEQ ID NO: 14, SEQ ID NO: 10, SEQ ID NO: 22, SEQ ID NO: 8, SEQ ID NO: 11, and SEQ ID NO: 19, respectively, to be screened for cell state specific expression.

FIG. 9 illustrates nucleotide preference at each nucleotide position in transcription factor binding polynucleotides for ESRRG, RORA, or RORB transcription factors.

FIG. 10 illustrates nucleotide preference at each nucleotide position in transcription factor binding polynucleotides for NFIA, NFIB, NFIC, or NFYC transcription factors.

FIG. 11 illustrates nucleotide preference at each nucleotide position in transcription factor binding polynucleotides for ESRRG transcription factor in human and mouse cell lines.

FIG. 12 illustrates a schematic of a vector for fine-tuned payload sequence expression utilizing transcriptional control (e.g., using an engineered promoter for cell state specific expression) and translational control (e.g., 5′UTR, 3′UTR, and coding region of the polynucleotide encoding the payload sequence).

FIG. 13 shows a violin plot of transcriptional activity of different promoters in induced pluripotent stem cells (iPSCs) expressing a mutant MeCP2 protein. Activation was compared for promoters SEQ ID NO: 133, SEQ ID NO: 137, SEQ ID NO: 140, SEQ ID NO: 132, SEQ ID NO: 139, and SEQ ID NO: 134. Individual points correspond to redundant barcodes for each promoter.

FIG. 14A shows a scatter plot comparing transcriptional activity of promoters containing a single transcription factor binding motif (“1 Match”) compared promoters containing two of the same transcription factor binding motif (“2 Matches”). Dark grey points denote the transcription factor binding motifs showing the highest activation when present at four copies (see FIG. 14C).

FIG. 14B shows a scatter plot comparing transcriptional activity of promoters containing two copies of a transcription factor binding motif (“2 Matches”) compared promoters containing three of the same transcription factor binding motif (“3 Matches”). Dark grey points denote the transcription factor binding motifs showing the highest activation when present at four copies (see FIG. 14C).

FIG. 14C shows a scatter plot comparing transcriptional activity of promoters containing three copies of a transcription factor binding motif (“3 Matches”) compared promoters containing four of the same transcription factor binding motif (“4 Matches”). Dark grey points denote the transcription factor binding motifs showing the highest activation when present at four copies.

FIG. 15A shows a scatter plot of transcriptional activity of duplicated pairs of transcription factor binding motifs as a function of the activity of the lowest activity transcription factor binding motif in each pair. The box denotes synergistic transcription factor binding motif pairs that exhibited higher activity than the individual motifs.

FIG. 15B shows a scatter plot of transcriptional activity of duplicated pairs of transcription factor binding motifs as a function of the activity of the highest activity transcription factor binding motif in each pair. The box denotes “lone wolf” transcription factor binding motifs that exhibited higher activity as individual motifs than when paired.

FIG. 16 shows a heatmap of transcriptional activation of specific transcription factor binding motif pairs when present in a promoter as duplicated pairs. Warmer colors indicate higher transcriptional activity.

FIG. 17A shows a scatter plot of transcriptional activity of duplicated pairs of transcription factor binding motifs as a function of the activity of the lowest activity transcription factor binding motif in each pair. Dark grey points denote motif pairs containing a RORB-binding motif.

FIG. 17B shows a scatter plot of transcriptional activity of duplicated pairs of transcription factor binding motifs as a function of the activity of the lowest activity transcription factor binding motif in each pair. Dark grey points denote motif pairs containing a NR1D1-binding binding motif.

FIG. 18A shows a sequence logo plot of NR1D1-binding motifs and individual NR1D1-binding motifs of SEQ ID NO: 71-SEQ ID NO: 75.

FIG. 18B shows a scatter plot of transcriptional activity of duplicated pairs of transcription factor binding motifs as a function of the activity of the highest activity transcription factor binding motif in each pair. Red points denote motif pairs containing a NR1D1-binding binding motif of SEQ ID NO: 72.

FIG. 19 shows sequence logo plots of ESRRG-binding motifs, RORA-binding motifs, and RORB-binding motifs along with individual RORB-binding motifs of SEQ ID NO: 88-SEQ ID NO: 92. RORB-binding motif sequences are ordered, from top to bottom, by decreasing match score to a consensus RORB-binding motif.

FIG. 20A shows a scatter plot of transcriptional activity of duplicated pairs of transcription factor binding motifs as a function of the activity of the highest activity transcription factor binding motif in each pair. Dark grey points denote motif pairs containing a NR1D1-binding binding motif. The circle indicates a promoter containing a duplicated transcription factor binding motif pair of a TCF7L2-binding motif and an NR1D1-binding motif.

FIG. 20B shows a violin plot of transcriptional activity in wild type induced pluripotent stem cells (iPSCs) of promoters containing a duplicated transcription factor binding motif pair of a TCF7L2-binding motif and an NR1D1-binding motif (SEQ ID NO: 138), four matched TCF7L2-binding motifs (SEQ ID NO: 135), or four matched NR1D1-binding motifs (SEQ ID NO: 136).

FIG. 21 shows a violin plot of fold change in transcriptional activity in induced pluripotent stem cells (iPSCs) expressing a mutant MeCP2 protein relative to wild type iPSCs of promoters containing rationally designed transcription factor binding polynucleotides of, from left to right, SEQ ID NO: 39, SEQ ID NO: 31, SEQ ID NO: 36, SEQ ID NO: 29, SEQ ID NO: 30, SEQ ID NO: 28, SEQ ID NO: 26, SEQ ID NO: 38, SEQ ID NO: 33, SEQ ID NO: 27, SEQ ID NO: 44, SEQ ID NO: 141, SEQ ID NO: 32, SEQ ID NO: 35, SEQ ID NO: 41, SEQ ID NO: 34, SEQ ID NO: 40, and SEQ ID NO: 37.

FIG. 22A shows a scatter plot of transcriptional activity for promoters containing a rationally described transcription factor binding polynucleotide of SEQ ID NO: 26 paired with different core promoters in wild type iPSCs versus iPSCs expressing a mutant MeCP2 protein. The circle indicates a promoter of SEQ ID NO: 115.

FIG. 22B shows a violin plot of transcriptional activity of a promoter of SEQ ID NO: 115 in iPSCs expressing a mutant MeCP2 protein, wild type iPSCs, mouse neurons expressing a mutant MeCP2 protein, or wild type mouse neurons.

FIG. 23 shows a scatter plot of transcriptional activity of promoters containing a transcription factor binding sequence of SEQ ID NO: 26 paired with twenty different core promoters compared to promoters containing a transcription factor binding sequence of SEQ ID NO: 38 paired with the same core promoters.

FIG. 24 shows a violin plot of transcriptional activity of 18 different transcription factor binding sequences paired with each of twenty different core promoters.

FIG. 25 schematically illustrates a workflow for performing a massively parallel reporter assay to identify cell type- or cell state-specific promoters.

### DETAILED DESCRIPTION

Described herein are polynucleotide compositions comprising a payload sequence under transcriptional control of a promoter. The polynucleotide compositions of the present disclosure may encode for transcription of the payload sequence at levels dependent on a cell state. In some embodiments, the polynucleotide compositions of the present disclosure are recombinant polynucleotides. In some embodiments, the level of transcription of the payload sequence may depend on a cell type (e.g., neuron, hepatocyte, retinal cell, epithelial cell, muscle cell, erythrocyte, platelet, bone marrow cell, endothelial cell, epidermal cell, lymphocyte, glial cell, interstitial cell, adipocyte, or fibroblast). In some embodiments, the level of transcription of the payload sequence may depend on a cell state, such as a cell genotype (e.g., presence or absence of one or more genetic mutations) or a cell phenotype (e.g., the presence or absence of the expression of one or more genetic mutations). In some embodiments, the level of transcription of the payload sequence may depend on both the cell type and the cell state. In some embodiments, the level of transcription of the payload sequence may depend on the cell type, the cell genotype, and the cell phenotype. The promoter may be selected or engineered to tune the level of transcription as well as the cell type- or cell state-dependence of payload sequence transcription. In some embodiments, tuning a transcription level may comprise adjusting transcription to a desired level. In some embodiments, the desired level may be cell type- or state-specific. In some embodiments, the desired level may be cell type- and state-specific. In some embodiments, tuning a transcription level may comprise selecting for a desired level of transcription in a cell state of interest. The transcription level of the payload sequence may control the expression level of a protein or nucleotide encoded by the payload sequence. For example, a high level of transcription of the payload sequence may lead to a high level of expression of the protein encoded by the payload sequence.

In some embodiments, the polynucleotide composition (e.g., a recombinant polynucleotide) may comprise a promoter. In embodiments, the promoter may comprise a transcription factor binding polynucleotide and a core promoter. In some embodiments, the transcription factor binding polynucleotide may be a recombinant transcription factor binding polynucleotide.

Also described herein are methods of delivering a polynucleotide composition (e.g., a recombinant polynucleotide) of the present disclosure to a subject. In some embodiments, the polynucleotide composition may be part of a viral vector capable of delivering the polynucleotide to a cell of the subject. The viral vector may comprise a viral inverted terminal repeat sequence that includes a viral origin of replication, enabling viral replication of the polynucleotide sequence. The viral vector may comprise a viral capsid encapsulating the polynucleotide and facilitating delivery of the polynucleotide into the cell. A method of delivering a polynucleotide composition (e.g., a recombinant polynucleotide of the present disclosure) to a subject may comprise administering a viral vector comprising the polynucleotide to the subject. Upon delivery of the polynucleotide to the subject, a payload sequence of the polynucleotide may be transcribed in a cell of the subject in a cell type- and/or cell state-dependent manner, resulting in expression of a protein or nucleotide encoded by the payload sequence in the target cell type and/or target cell state.

Further described herein are methods of treating a disease or condition by delivering a polynucleotide composition (e.g., a recombinant polynucleotide) of the present disclosure to a subject and expressing a protein or nucleotide encoded by the polynucleotide in the subject in a cell type- and/or cell state-dependent manner. The polynucleotide composition may be delivered to the subject as part of a viral vector. The subject may have a disease or condition, for example a disease or condition caused by mutation or having altered expression of a gene. In some embodiments, a payload sequence of the polynucleotide composition may be a transgene encoding a wild type copy of a protein encoded by the gene with the mutation or having altered expression. The transgene may be transcribed in a cell of the subject in a cell state-dependent manner upon delivery of the polynucleotide composition to the subject. In some embodiments, a protein encoded by the transgene is expressed in the subject at a level dependent on the level of transcription of the transgene. Transcription of the transgene, expression of the protein encoded by the transgene, or both, in a cell state-dependent manner may treat the disease or condition in the subject. Alternately or in addition, the payload sequence of the polynucleotide composition (e.g., a recombinant polynucleotide) may encode a therapeutic polynucleotide (e.g., a gRNA or tRNA) that targets the gene with the mutation or altered expression. The therapeutic polynucleotide may be transcribed in a cell of the subject in a cell state-dependent and/or cell type-dependent manner upon delivery of the polynucleotide composition to the subject. In some embodiments, the therapeutic polynucleotide encoded by the payload sequence is expressed in the subject at a level dependent on the level of transcription of the payload sequence. Transcription of the payload sequence, expression of the therapeutic polynucleotide encoded by the payload sequence, or both, in a cell state-dependent and/or cell type-dependent manner may treat the disease or condition in the subject.

### Promoters

A polynucleotide (e.g., an RNA or a DNA polynucleotide) may comprise a promoter sequence to regulate or enhance transcription of a payload sequence (e.g., a transgene or a therapeutic polynucleotide) under transcriptional control of the promoter. In some embodiments, the polynucleotide may be a recombinant polynucleotide. In some embodiments, the polynucleotide may comprise a transcription factor (TF) binding polynucleotide that binds one or more transcription factors, coactivators, or corepressors, and a core promoter that functions as a site for preinitiation complex formation. In some embodiments, the sequence of the promoter may comprise a transcription factor (TF) binding sequence that binds one or more transcription factors, coactivators, or corepressors, and a core promoter sequence that functions as a site for preinitiation complex formation. The elements within the promoter sequence (e.g., the transcription factor binding sequence and the core promoter sequence) may be engineered to alter transcription rates of a downstream payload sequence. In some embodiments, the promoter may be engineered for cell type- and/or cell state-specific transcription. For example, the promoter may be engineered to promote high levels of transcription in target cell type (e.g., neurons) and low levels or no transcription in non-target cell types (e.g., non-neuronal cells). In another example, the promoter may be engineered to promote high levels of transcription in cells having a disease phenotype caused by a genetic mutation or variation (e.g., a genetic mutation or variation associated with a disease or a condition) and low levels or no transcription in cells lacking the disease phenotype.

In some embodiments, a promoter may promote cell type- and/or cell state-specific transcription if it promotes transcription of a payload sequence in a target cell type and/or target cell state at a level that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the transgene in a non-target cell type and/or non-target cell state. A promoter as disclosed herein may be a recombinant promoter.

### Transcription Factor Binding Sequences

The promoter of a polynucleotide may comprise a transcription factor binding polynucleotide that binds one or more transcription factors, coactivators, or corepressors to modulate transcription of a nearby polynucleotides. The promoter sequence of a polynucleotide may comprise a transcription factor binding polynucleotide sequence that binds one or more transcription factors, coactivators, or corepressors to modulate transcription of nearby polynucleotides. The transcription factor binding polynucleotide may recruit transcription factors to the polynucleotide that enhance, repress, or alter transcription of a downstream sequence (e.g., a transgene encoded by the polynucleotide). A transcription factor binding polynucleotide as disclosed herein may be a recombinant transcription factor binding polynucleotide. In some embodiments, the transcription factor binding polynucleotide may comprise one or more transcription factor binding motifs, each of which binds a transcription factor. Transcriptional enhancement, cell type-specificity, and/or cell state-specificity may be tuned by including different combinations, orientations, or variants of transcription factor binding motifs in the transcription factor binding polynucleotide.

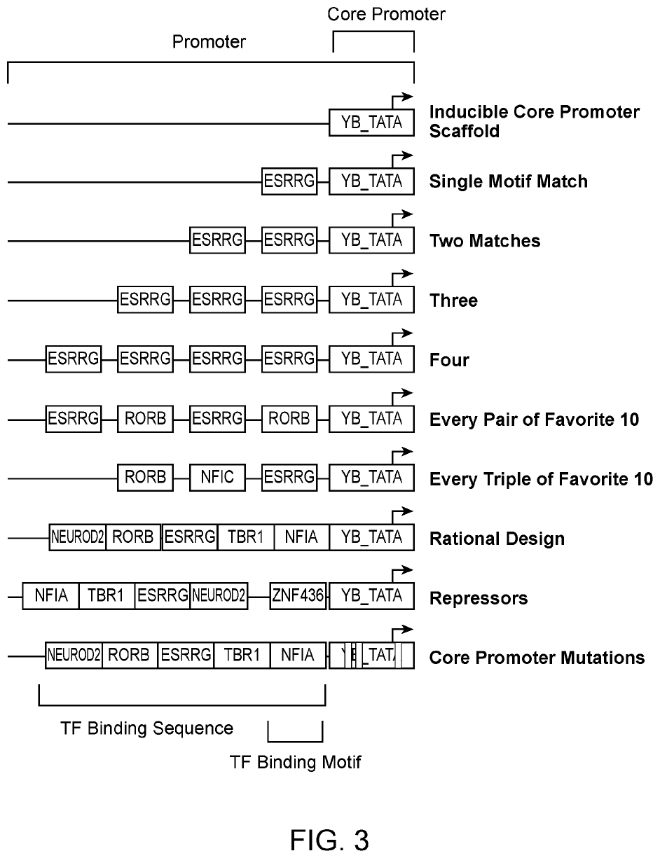

Examples of ways in which transcription factor binding motifs may be combined in a transcription factor binding sequence of a promoter are illustrated in FIG. 3. In some embodiments, a transcription factor binding motif may be duplicated one, two, three, four, or more times to enhance recruitment of the transcription factor that binds the transcription factor binding motif. In some embodiments, two, three, four, five, six, seven, eight, or more different transcription factor binding motifs may be combined in a transcription factor binding sequence to recruit two, three, four, five, six, seven, eight, or more different transcription factors. In some embodiments, a transcription factor binding motif may bind a transcriptional enhancer (e.g., a transcription factor that enhances or increases transcription of a downstream sequence compared to transcription in the absence of the transcription factor binding motif). In some embodiments, a transcription factor binding motif may bind a transcriptional repressor (e.g., a transcription factor that represses or decreases transcription of a downstream sequence compared to transcription in the absence of the transcription factor binding motif).

A transcription factor binding polynucleotide (e.g., a recombinant transcription factor binding polynucleotide) may be engineered for one or more desired transcriptional properties, such as transcription level, cell type specificity, cell genotype specificity, and/or cell phenotype. For example, a transcription factor binding polynucleotide may be engineered to promote a moderate level of transcription in neurons with a phenotype resulting from a genetic mutation and little to no transcription in non-neuronal cell types and neurons lacking the phenotype resulting from the genetic mutation. Engineering a transcription factor binding polynucleotide for cell state specific transcription may comprise selecting or screening for transcription factors that are expressed in a cell state of interest and incorporating one or more transcription factor binding motifs that bind to the identified transcription factors into the transcription factor binding polynucleotide. For example, a transcription factor binding polynucleotide with neuron-specific transcription may comprise one or more transcription factor binding motifs that bind one or more transcription factors expressed in neurons. Examples of transcription factors expressed in neurons are provided in TABLE 1. In some embodiments, cell state specificity may be further tuned using identified transcription factors that are expressed at increased levels in a target cell state (e.g., a cell type of interest, a cell genotype of interest, and/or a cell phenotype of interest) relative to a non-target cell state. For example, a transcription factor binding polynucleotide with enhanced transcription levels in neurons relative to hepatocytes may comprise one or more transcription factor binding motifs that bind one or more transcription factors expressed more highly in neurons than in hepatocytes. Examples of empirically determined neuron to hepatocyte expression ratios of transcription factors expressed in neurons are provided in TABLE 1.

TABLE 1
Exemplary Transcription Factors with Neuronal Expression

| Transcription Factor Gene | Neuron to Hepatocyte Expression Ratio |
|---|---|
| ZNF436 | 2.21 |
| NR4A1 | 0.83 |
| IRF8 | 0.01 |
| ZBTB18 | 17.64 |
| NRII3 | 0.01 |
| ETV5 | 0.64 |
| SOX2 | 28.68 |
| JUNB | 0.13 |
| ZNF563 | 0.07 |
| PPARA | 0.05 |
| MEF2C | 60.82 |
| NEUROD2 | 121.12 |
| FOS | 0.73 |
| TCF4 | 37.34 |
| HLF | 0.17 |
| MAF | 0.07 |
| LHX2 | 71.71 |
| PBX1 | 4.73 |
| FOXP1 | 0.49 |
| ZBTB7A | 0.40 |
| CUX1 | 0.70 |
| FOXO3 | 0.62 |
| POU3F2 | 116.73 |
| NFYC | 0.48 |
| NR3C1 | 0.06 |
| BCL6 | 0.47 |
| ZEB1 | 2.09 |
| TCF3 | 0.99 |
| NR1D1 | 0.55 |
| ZFP28 | 4.14 |
| ETS2 | 0.13 |
| STAT1 | 0.24 |
| POU3F1 | 62.31 |
| ZBTB33 | 0.95 |
| MXI1 | 2.20 |
| NFIC | 0.59 |
| ETS1 | 0.13 |
| VEZF1 | 2.06 |
| KLF3 | 0.51 |
| ZNF250 | 4.11 |
| MAFB | 0.19 |
| NFIA | 3.94 |
| RFX5 | 0.73 |
| BHLHE40 | 0.29 |
| KLF12 | 0.53 |
| STAT4 | 0.20 |
| ETV1 | 1.51 |
| RORA | 0.12 |
| MITF | 0.79 |
| NFE2L2 | 0.20 |
| ESRRG | 12.29 |
| PBX3 | 0.95 |
| TCF7L2 | 0.15 |
| NKX3-1 | 0.04 |
| NR1H4 | 0.00 |
| ONECUT1 | 0.00 |
| FOXA3 | 0.00 |
| EMX1 | 117.31 |
| FOXG1 | 67.80 |
| BHLHE41 | 2.90 |
| ISX | 0.00 |
| ZBTB7C | 2.65 |
| OTX1 | 119.38 |
| PITX3 | 2.45 |
| NR3C2 | 0.21 |
| EGR4 | N/A |
| SCRT1 | N/A |
| CUX2 | 16.67 |
| ONECUT2 | 0.08 |
| POU6F2 | 14.19 |
| RFX4 | 16.63 |
| TBR1 | 51.44 |
| HES5 | 4.21 |
| XBP1 | 0.04 |
| SOX11 | 81.53 |
| DLX1 | 9.35 |
| RORB | 75.19 |
| FOXN3 | 0.71 |
| NR1D2 | 0.66 |
| SMAD5 | 0.61 |
| PLAGL1 | 8.94 |
| NFIB | 42.71 |
| BBX | 0.50 |
| DPF1 | 68.62 |
| YBX1 | 1.26 |
| ZBTB20 | 0.38 |
| ZNF177 | 0.20 |
| ZNF385D | 16.15 |
| ZNF189 | 0.53 |

The transcription level, cell type specificity, and/or cell state specificity of a transcription factor binding polynucleotide (e.g., a recombinant transcription factor binding polynucleotide) may be further tuned by varying the sequence of one or more transcription factor binding motifs to alter the affinity to the corresponding transcription factor. In some embodiments, a transcription factor may have a preferred binding sequence (also referred to herein as a “consensus transcription factor binding motif” or a “consensus motif”). The preferred binding sequence may bind the transcription factor with higher affinity than other binding motifs or variants of the binding motif. In some embodiments, a consensus motif may increase recruitment of the corresponding transcription factor relative to other binding motifs or variants of the binding motif. Transcription levels may be tuned by introducing sequence variations into a consensus motif to alter affinity of the motif for the transcription factor. For example, to tune a level of transcription and prevent over-expression of a payload, a transcription factor binding motif comprising one or more sequence variations relative to a consensus transcription factor binding motif may be included in the transcription factor binding polynucleotide. The transcription factor binding polynucleotide comprising the variant transcription factor binding motif may promote reduced transcription of a payload sequence relative to a transcription factor binding sequence comprising a consensus transcription factor binding motif.

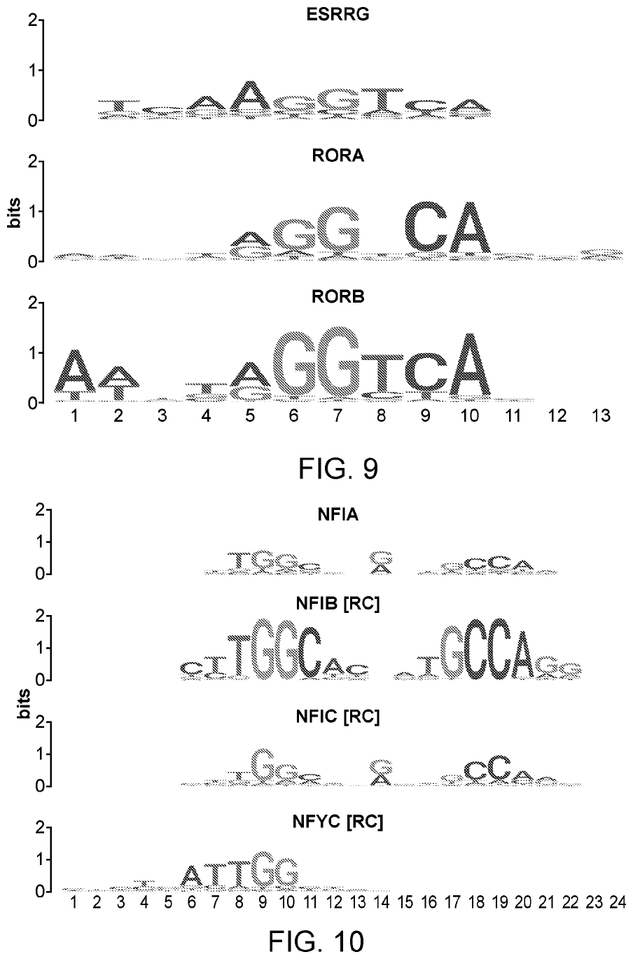

In some embodiments, a variant transcription factor binding motif may comprise at least about 40%, at least about 50%, at least about 60%, at least about 70%, at least about 80%, at least about 90%, at least about 95%, or at least about 98% sequence identity to a consensus transcription factor binding motif. In some embodiments, a variant transcription factor binding motif may comprise no more than about 40%, no more than about 50%, no more than about 60%, no more than about 70%, no more than about 80%, no more than about 90%, no more than about 95%, no more than about 98% sequence identity to a consensus transcription factor binding motif. Examples of sequence preferences of select transcription factors are illustrated in FIG. 9 and FIG. 10. The size of the letter corresponding to a nucleotide corresponds with the degree of preference of the transcription factor for the nucleotide at the indicated position.

Examples of transcription factor binding motifs that may be included in a transcription factor binding motif to promote cell type- or cell state-specific expression of a payload sequence are provided in TABLE 2. These transcription factor binding motifs may promote cell type- or cell-state specific transcription in a corresponding cell state or cell type. In some embodiments, a transcription factor binding motif may comprise at least about 40%, at least about 50%, at least about 60%, at least about 70%, at least about 80%, at least about 85%, at least about 87%, at least about 90%, at least about 93%, at least about 95%, or at least about 98%, or about 100% sequence identity to a transcription factor binding motif provided in TABLE 2.

TABLE 2
Exemplary Transcription Factor Binding Motifs

| Transcription | | |
|---|---|---|
| Factor | SEQ ID NO | Sequence |
| CUX1 | SEQ ID NO: 45 | AGGGGGATCGATGG |
| CUX1 | SEQ ID NO: 46 | ATATGTATTTGTTA |
| ESRRG | SEQ ID NO: 47 | TCAAGGTCA |
| ESRRG | SEQ ID NO: 48 | TGGGGGACA |
| ETV5 | SEQ ID NO: 49 | GAGCAGGAAGTGAG |
| ETV5 | SEQ ID NO: 50 | GGGCAGAAGGCGGA |
| IRF8 | SEQ ID NO: 51 | AAAAGAGGAAGTGAAAGTAA |
| IRF8 | SEQ ID NO: 52 | CACCAGGGAAATGAGCGTGC |
| KLF12 | SEQ ID NO: 53 | AGGGGCGGGGC |
| KLF12 | SEQ ID NO: 54 | TGGGGCGGGTA |
| TFDP1 | SEQ ID NO: 55 | GGCAGCGGGTAC |
| TFDP1 | SEQ ID NO: 56 | GGCAGAGGAGAC |
| ZFP57 | SEQ ID NO: 57 | TGCCGCAGCGGC |
| ZFP57 | SEQ ID NO: 58 | TGCCGCAGCGCG |
| ZFP57 | SEQ ID NO: 59 | CCGCTGGGGCAG |
| ZFP57 | SEQ ID NO: 60 | CCGCTCCGGCAT |
| TEAD4 | SEQ ID NO: 61 | AGGAATACGGAG |
| TEAD4 | SEQ ID NO: 62 | TGCAATAAGGAG |
| NEUROD1 | SEQ ID NO: 63 | ACCGACGGACGG |
| NEUROD1 | SEQ ID NO: 64 | GCCGACGGAGGG |
| NEUROD2 | SEQ ID NO: 65 | AACAGATGG |
| NEUROD2 | SEQ ID NO: 66 | GGGAGATGG |
| NFIA | SEQ ID NO: 67 | TTGGCACGGTGCCAA |
| NFIA | SEQ ID NO: 68 | CTGAGCCGGTACCCT |
| NFIC | SEQ ID NO: 69 | CTTGGCTCCCTGCCAAG |
| NFIC | SEQ ID NO: 70 | GTTGCCTGTTTCCCCCG |
| NR1D1 | SEQ ID NO: 71 | TACAAGCTTTGTCACCGGT |
| NR1D1 | SEQ ID NO: 72 | GGAAATTTGGGACAGAAGA |
| NR1D1 | SEQ ID NO: 73 | AGGGAAGTGGGTCAGAAGG |
| NR1D1 | SEQ ID NO: 74 | AAGCAAGTGGGTCAGAAGG |
| NR1D1 | SEQ ID NO: 75 | AGCAAACTGGGCCTATCGA |
| NR1D1 | SEQ ID NO: 76 | GGCTATCTTGGGCATTCGG |
| ONECUT2 | SEQ ID NO: 77 | AAAAAATCGATAAT |
| ONECUT2 | SEQ ID NO: 78 | TATAAATCAATAAG |
| PBX3 | SEQ ID NO: 79 | TGATTGACAGG |
| PBX3 | SEQ ID NO: 80 | TGAGTGATCCG |
| PPARA | SEQ ID NO: 81 | AACTGGGGCAAAGGTCA |
| PPARA | SEQ ID NO: 82 | AGCTGGATCAAATAACA |
| RFX4 | SEQ ID NO: 83 | CGTTGCCATGGCAACG |
| RFX4 | SEQ ID NO: 84 | AGCAACCATGGTTACG |
| RORA | SEQ ID NO: 85 | AACTAGGTCAGGG |
| RORA | SEQ ID NO: 86 | AGTTCGGGCACTG |
| RORB | SEQ ID NO: 87 | AAATGGGTGAT |
| RORB | SEQ ID NO: 88 | AATTAGGTCAC |
| RORB | SEQ ID NO: 89 | AAATAGGTCAG |
| RORB | SEQ ID NO: 90 | AACCAGGTCAA |
| RORB | SEQ ID NO: 91 | ACTTAGGTCAA |
| RORB | SEQ ID NO: 92 | TTGTATGTCAT |
| TBR1 | SEQ ID NO: 93 | AGGTGTGAAA |
| TBR1 | SEQ ID NO: 94 | GGGTGTTAAA |
| TCF7L2 | SEQ ID NO: 95 | CCCTTTGATGTGG |
| TCF7L2 | SEQ ID NO: 96 | CGCTCTGAAATAG |
| ZBTB7C | SEQ ID NO: 97 | GCGACCACCGAA |
| ZBTB7C | SEQ ID NO: 98 | GCAACCCCCTCC |
| ZNF436 | SEQ ID NO: 99 | TCAGGGAAGGCTTCCTGGAGG |
| | | AGG |
| ZNF436 | SEQ ID NO: 100 | TCTTGAAGCACTCCCTGGAAG |
| | | AGG |
| XBP1 | SEQ ID NO: 101 | ATGGACACGTCACC |
| BCL6 | SEQ ID NO: 102 | TGCTTTCCAGGAA |
| HES5 | SEQ ID NO: 103 | CGGCACGTGCCA |
| NFIC | SEQ ID NO: 104 | CTTGGTTCCGTTCCACT |
| TCF7L2 | SEQ ID NO: 105 | TCCTTTGATGACG |
| SOX11 | SEQ ID NO: 106 | AACAATTGCATTGTT |
| BCL6 | SEQ ID NO: 107 | CCCTTTCCAAGAA |
| HES5 | SEQ ID NO: 108 | CGGCACGTGCCG |
| TCF7L2 | SEQ ID NO: 109 | CCCTTTGATGTTG |
| NEUROD2 | SEQ ID NO: 110 | CACAGATGG |
| HES5 | SEQ ID NO: 111 | TGGCACGTGCCA |
| ESRRG | SEQ ID NO: 112 | TCAAGCTCG |

In some embodiments, a sequence of a transcription factor binding polynucleotide (e.g., a recombinant transcription factor binding polynucleotide) may comprise one or more transcription factor binding motifs that bind to one or more of a ZNF436, NR4A1, IRF8, ZBTB18, NR113, ETV5, SOX2, JUJNB, ZNF563, PPARA, MEF2C, NEUIROD1, NEUIROD2, FOS, TCF4, HLF, MAF, LHX2, PBX1, FOXP1, ZBTB7A, CUX1, FOX03, POU3F2, NFYC, NR3C1, BCL6, ZEB1, TCF3, NR1D1, ZFP28, ZFP57, ETS2, STAT1, POU3F1, ZBTB33, MXI1, NFIC, ETS1, VEZF1, KLF3, ZNF250, MAFB, NFIA, RFX5, BHLHE40, KLF12, STAT4, ETV1, RORA, MITF, NFE2L2, ESRRG, PBX3, TCF7L2, NKX3-1, NR1H4, ONECUT1, FOXA3, EMX1, FOXG1, BHLHE41, ISX, ZBTB7C, OTX1, PITX3, NR3C2, EGR4, SCRT1, CUX2, ONECUT2, POU6F2, RFX4, TBR1, HES5, XBP1, SOX11, DLX1, RORB, FOXN3, NR1D2, SMAD5, PLAGL1, NFIB, BBX, DPF1, TFDP1, TEAD4, YBX1, ZBTB20, ZNF177, ZNF385D, or a ZNF189 transcription factor, or combinations thereof. In some embodiments, a transcription factor binding sequence may comprise one or more transcription factor binding motifs that bind to a transcription factor differentially expressed in a target cell type (e.g., neurons, hepatocytes, retinal cells, epithelial cells, muscle cells, erythrocytes, platelets, bone marrow cells, endothelial cells, epidermal cells, lymphocytes, glial cells, interstitial cells, adipocytes, fibroblasts, or combinations thereof). In some embodiments, a transcription factor binding sequence may comprise one or more transcription factor binding motifs that bind to a transcription factor differentially expressed in a cell with a genotype of interest (e.g., a genotype associated with a disease or condition). In some embodiments, a transcription factor binding sequence may comprise one or more transcription factor binding motifs that bind to a transcription factor differentially expressed in a cell with a phenotype of interest (e.g., a phenotype resulting from a genotype associated with a disease or condition).

A transcription factor binding motif sequence, such as a sequence that binds to a transcription factor listed in TABLE 1, may comprise an endogenous transcription factor binding motif sequence. In some embodiments, the transcription factor binding motif sequence may be engineered based on an endogenous transcription factor binding motif sequence. For example, an engineered transcription factor binding motif sequence may have at least about 40%, at least about 50%, at least about 60%, at least about 70%, at least about 80%, at least about 85%, at least about 87%, at least about 90%, at least about 93%, at least about 95%, or at least about 98%, or about 100% sequence identity to an endogenous transcription factor binding motif. Alternatively or in addition, the transcription factor binding motif sequence may be a synthetic transcription factor binding motif sequence that is engineered de novo to enhance transcription in a cell type- and/or cell state-specific manner. In some embodiments, the synthetic transcription factor binding motif sequence may be engineered to bind a transcription factor (e.g., a transcription factor listed in TABLE 1).

In some embodiments, the transcription factor binding motif may be a consensus transcription factor binding motif. In some embodiments, the transcription factor binding motif may be a variant transcription factor binding motif. In some embodiments, the transcription factor binding motif may be a reverse complement of a consensus transcription factor binding motif or a reverse complement of a variant transcription factor binding motif.

A workflow for tuning the transcription level and cell state specificity of a transcription factor binding sequence may comprise identifying transcription factors that are differentially expressed in a cell state of interest, generating candidate transcription factor binding sequences comprising combinations, duplications, reverse complements, or variants of transcription factor binding motifs that bind to the identified transcription factors, and screening the candidate transcription factor binding sequences for transcription level and cell state specificity. In some embodiments, a library of polynucleotides comprising different transcription factor binding sequences may be screened for transcription level and cell state specificity. In some embodiments, a library of polynucleotides comprising different transcription factor binding sequences and different core promoters may be screened for transcription level and cell state specificity of the promoter.

A workflow for tuning the transcription level and cell type specificity of a transcription factor binding sequence may comprise identifying transcription factors that are differentially expressed in a cell type of interest, generating candidate transcription factor binding sequences comprising combinations, duplications, reverse complements, or variants of transcription factor binding motifs that bind to the identified transcription factors, and screening the candidate transcription factor binding sequences for transcription level and cell type specificity. In some embodiments, a library of polynucleotides comprising different transcription factor binding sequences may be screened for transcription level and cell type specificity. In some embodiments, a library of polynucleotides comprising different transcription factor binding sequences and different core promoters may be screened for transcription level and cell type specificity of the promoter.

A workflow for tuning the transcription level and cell phenotype of a transcription factor binding sequence may comprise identifying transcription factors that are differentially expressed in a cell phenotype of interest, generating candidate transcription factor binding sequences comprising combinations, duplications, reverse complements, or variants of transcription factor binding motifs that bind to the identified transcription factors, and screening the candidate transcription factor binding sequences for transcription level and cell phenotype specificity. In some embodiments, a library of polynucleotides comprising different transcription factor binding sequences may be screened for transcription level and cell phenotype specificity. In some embodiments, a library of polynucleotides comprising different transcription factor binding sequences and different core promoters may be screened for transcription level and cell phenotype specificity of the promoter.

A workflow for tuning the transcription level, cell type, and cell phenotype of a transcription factor binding sequence may comprise identifying transcription factors that are differentially expressed in a cell type and in a cell phenotype of interest, generating candidate transcription factor binding sequences comprising combinations, duplications, reverse complements, or variants of transcription factor binding motifs that bind to the identified transcription factors, and screening the candidate transcription factor binding sequences for transcription level, cell type specificity, and cell phenotype specificity. In some embodiments, a library of polynucleotides comprising different transcription factor binding sequences may be screened for transcription level, cell type specificity, and cell phenotype specificity. In some embodiments, a library of polynucleotides comprising different transcription factor binding sequences and different core promoters may be screened for transcription level, cell type specificity, and cell phenotype specificity of the promoter.

A workflow for tuning the transcription level, cell type, and cell genotype of a transcription factor binding sequence may comprise identifying transcription factors that are differentially expressed in a cell type and in a cell genotype of interest, generating candidate transcription factor binding sequences comprising combinations, duplications, reverse complements, or variants of transcription factor binding motifs that bind to the identified transcription factors, and screening the candidate transcription factor binding sequences for transcription level, cell type specificity, and cell genotype specificity. In some embodiments, a library of polynucleotides comprising different transcription factor binding sequences may be screened for transcription level, cell type specificity, and cell genotype specificity. In some embodiments, a library of polynucleotides comprising different transcription factor binding sequences and different core promoters may be screened for transcription level, cell type specificity, and cell genotype specificity of the promoter.

The transcription factor binding motifs described herein (e.g., a transcription factor binding motif provided in TABLE 2 or a transcription factor binding motif that binds to a transcription factor provided in TABLE 1) may be combined to form a transcription factor binding sequence of a transcription factor binding polynucleotide (e.g., a recombinant transcription factor binding polynucleotide). In some embodiments, a transcription factor binding sequence may comprise 1, 2, 3, 4, 5, 6, 7, 8, 9, or 10 transcription factor binding motifs. For example, a transcription factor binding sequence may comprise three transcription factor binding motifs. For example, a transcription factor binding sequence may comprise four transcription factor binding motifs. For example, a transcription factor binding sequence may comprise five transcription factor binding motifs. Examples of transcription factor binding sequences that may promote cell type- or cell state-specific expression are provided in TABLE 3. In some embodiments, a transcription factor binding sequence may comprise at least about 40%, at least about 50%, at least about 60%, at least about 70%, at least about 80%, at least about 85%, at least about 87%, at least about 90%, at least about 93%, at least about 95%, or at least about 98%, or about 100% sequence identity to a transcription factor binding motif sequence in TABLE 3. In some embodiments, a transcription factor binding sequence may comprise at least 9Ce sequence identity to SEQ ID NO: 26. In some embodiments, a transcription factor binding sequence may comprise at least 9300 sequence identity to SEQ ID NO: 26. In some embodiments, a transcription factor binding sequence may comprise at least 950 sequence identity to SEQ ID NO: 26. In some embodiments, a transcription factor binding sequence may comprise at least 98A sequence identity to SEQ ID NO: 26. In some embodiments, a transcription factor binding sequence may comprise SEQ ID NO: 26.

TABLE 3
Exemplary Transcription Factor Binding Sequences and Corresponding Cell
State or Cell Type

| SEQ ID | | Cell |
|---|---|---|
| NO | Sequence | Type/State |
| SEQ ID | ACCGACGGACGGGGCAGCGGGTACCCGCTGGGGCAGT | MeCP2 |
| NO: 26 | GCCGCAGCGGCAGGAATACGGAGGGCAGCGGGTACCC | mutant |
| | GCTGGGGCAGTGCCGCAGCGGCAGGAATACGGAG | neurons |
| SEQ ID | CACCAGGGAAATGAGCGTGCGGGCAGAAGGCGGAAGC | MeCP2 |
| NO: 27 | AACCATGGTTACGATATGTATTTGTTAGGGCAGAAGGC | mutant |
| | GGAAGCAACCATGGTTACGATATGTATTTGTTA | neurons |
| SEQ ID | AGCTGGATCAAATAACAAGCTGGATCAAATAACACGCT | MeCP2 |
| NO: 28 | CTGAAATAGCGCTCTGAAATAGTGGGGGGGGTATGGGG | mutant |
| | CGGGTATATAAATCAATAAGTATAAATCAATAAG | neurons |
| SEQ ID | TCTTGAAGCACTCCCTGGAAGAGGTGGGGGACAAAATG | MeCP2 |
| NO: 29 | GGTGATTGGGGGACAAGTTCGGGCACTGTGGGGGACAA | mutant |
| | GTTCGGGCACTGTGGGGGACAAGTTCGGGCACTG | neurons |
| SEQ ID | AACTGGGGCAAAGGTCAAACTGGGGCAAAGGTCACCCT | MeCP2 |
| NO: 30 | TTGATGTGGCCCTTTGATGTGGAGGGGCGGGGCAGGGG | mutant |
| | CGGGGCAAAAAATCGATAATAAAAAATCGATAAT | neurons |
| SEQ ID | GGGAGATGGGGGAGATGGGGGTGTTAAAGGGTGTTAA | MeCP2 |
| NO: 31 | ATGGGGGACATGGGGGACACTGAGCCGGTACCCTGTTG | mutant |
| | CCTGTTTCCCCCGCGCTCTGAAATAG | neurons |
| SEQ ID | CTTGGCTCCCTGCCAAGCTTGGCTCCCTGCCAAGTTGGC | MeCP2 |
| NO: 32 | ACGGTGCCAATTGGCACGGTGCCAACCCTTTGATGTGG | mutant |
| | CCCTTTGATGTGGAGGTGTGAAAAGGTGTGAAA | neurons |
| SEQ ID | AAAAGAGGAAGTGAAAGTAAGAGCAGGAAGTGAGCGT | MeCP2 |
| NO: 33 | TGCCATGGCAACGAGGGGGATCGATGGGAGCAGGAAG | mutant |
| | TGAGCGTTGCCATGGCAACGAGGGGGATCGATGG | neurons |
| SEQ ID | TGGGGGACATGGGGGACATGAGTGATCCGTGAGTGATC | MeCP2 |
| NO: 34 | CGCGCTCTGAAATAGCGCTCTGAAATAGTGAGTGATCC | mutant |
| | GCGCTCTGAAATAGTGGGGGACATGGGGGACA | neurons |
| SEQ ID | GTTGCCTGTTTCCCCCGGTTGCCTGTTTCCCCCGCTGAG | MeCP2 |
| NO: 35 | CCGGTACCCTCTGAGCCGGTACCCTCGCTCTGAAATAGC | mutant |
| | GCTCTGAAATAGGGGTGTTAAAGGGTGTTAAA | neurons |
| SEQ ID | TCAGGGAAGGCTTCCTGGAGGAGGTCAAGGTCAAATTA | MeCP2 |
| NO: 36 | GGTCACTCAAGGTCAAACTAGGTCAGGGTCAAGGTCAA | mutant |
| | ACTAGGTCAGGGTCAAGGTCAAACTAGGTCAGGG | neurons |
| SEQ ID | TACAAGCTTTGTCACCGGTGTTGCCTGTTTCCCCCGGCA | MeCP2 |
| NO: 37 | ACCCCCTCCTGGGGGACACGCTCTGAAATAGGCAACCC | mutant |
| | CCTCCTGGGGGACACGCTCTGAAATAG | neurons |
| SEQ ID | GCCGACGGAGGGGGCAGAGGAGACCCGCTCCGGCATT | MeCP2 |
| NO: 38 | GCCGCAGCGCGTGCAATAAGGAGGGCAGAGGAGACCC | mutant |
| | GCTCCGGCATTGCCGCAGCGCGTGCAATAAGGAG | neurons |
| SEQ ID | AACAGATGGAACAGATGGAGGTGTGAAAAGGTGTGAA | MeCP2 |
| NO: 39 | ATCAAGGTCATCAAGGTCATTGGCACGGTGCCAACTTG | mutant |
| | GCTCCCTGCCAAGCCCTTTGATGTGG | neurons |
| SEQ ID | AGGGAAGTGGGTCAGAAGGCTTGGCTCCCTGCCAAGGC | MeCP2 |
| NO: 40 | GACCACCGAATCAAGGTCACCCTTTGATGTGGGCGACC | mutant |
| | ACCGAATCAAGGTCACCCTTTGATGTGG | neurons |
| SEQ ID | TCAAGGTCATCAAGGTCATGATTGACAGGTGATTGACA | MeCP2 |
| NO: 41 | GGCCCTTTGATGTGGCCCTTTGATGTGGTGATTGACAGG | mutant |
| | CCCTTTGATGTGGTCAAGGTCATCAAGGTCA | neurons |

The transcription factor binding sequences (e.g., the transcription factor binding sequences provided in TABLE 3 or a transcription factor binding sequence comprising a one or more transcription motifs described herein) may be combined with a core promoter and a payload sequence to form a polynucleotide (e.g., a recombinant polynucleotide) construct for cell type- and/or cell state-specific expression of the payload sequence.

### Core Promoters

The promoter of a polynucleotide may comprise a core promoter that facilitates recruitment of transcription machinery and initiation of transcription. The promoter sequence of a polynucleotide may comprise a core promoter sequence that facilitates recruitment of transcription machinery and initiation of transcription. In some embodiments, the core promoter sequence may be positioned downstream (i.e., 3′) of the transcription factor binding polynucleotide sequence. In some embodiments, the core promoter sequence may be positioned upstream (i.e., 5′) of a payload sequence. The core promoter may recruit polymerases, co-factors, or proteins that bind to polymerases to initiate transcription of a sequence downstream of the core promoter. For example, the core promoter sequence may recruit an RNA polymerase (e.g., RNA polymerase II) or a TATA binding protein (TBP) that recruits an RNA polymerase when in combination with a response element (e.g., a transcription factor binding sequence) bound to cognate ligands (e.g., transcription factors), coactivators, or corepressors. The ability of the core promoter sequence to recruit transcription machinery (e.g., an RNA polymerase) or the affinity of the core promoter sequence for the transcription machinery may affect transcription levels. In some embodiments, the core promoter sequence may be altered to tune transcription levels by altering recruitment of or affinity for transcription machinery.

Core promoter sequences may be engineered for one or more desired transcriptional properties, such as transcription level, cell type specificity, and/or cell genotype specificity. For example, a core promoter sequence may be engineered to promote a moderate level of transcription in neurons with a genetic mutation and little to no transcription in non-neuronal cell types and neurons lacking the genetic mutation. Engineering a core promoter sequence may comprise screening variants of a core promoter sequence for transcription level, cell type specificity, or cell genotype specificity.

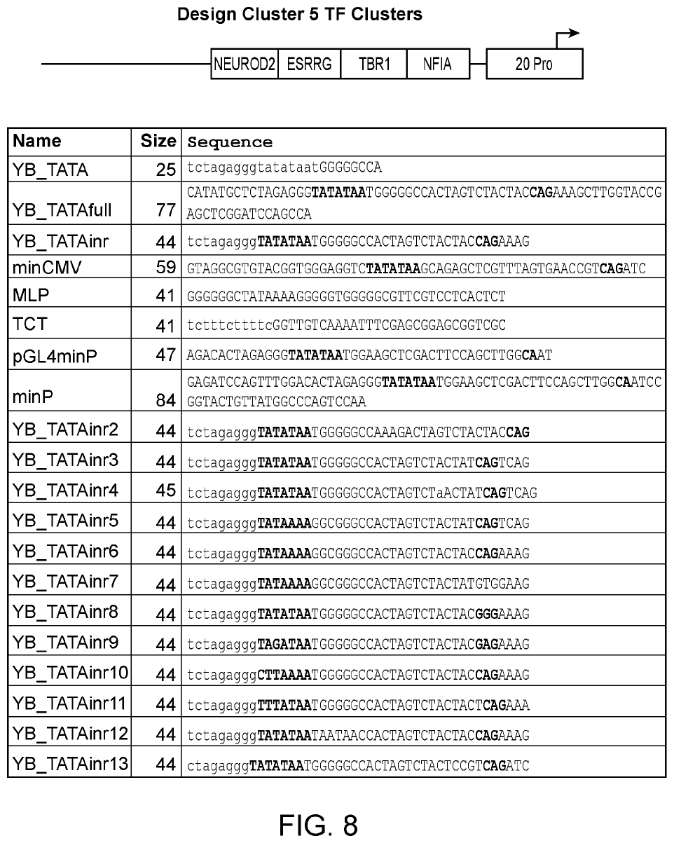

In some embodiments, a core promoter sequence may comprise a TATA box (e.g., TATAAA), an RNA polymerase binding sequence, a B recognition element (BRE, e.g., G/C,G/C,G/A,CGCC), a CCAAT box or CAT box (e.g., GGCCAATCT), or a Pribnow box (e.g., TATAAT). Examples of core promoter sequences are provided in in FIG. 8. Additional examples of core promoter sequences are provided in TABLE 4.

TABLE 4
Exemplary Core Promoter Sequences

| Core Promoter | SEQ ID NO | Sequence |
|---|---|---|
| TATA | SEQ ID | TATAAAAG |
| | NO: 1 | |
| CMV53 | SEQ ID | CAACAAAATGTCGTAACAAGGGCGGTAGGCGTGTACGGTG |
| | NO: 2 | GGAGGTCTATATAAGCAGAGCTCGTTTAGTGAACCG |
| minSV40 | SEQ ID | TGCATCTCAATTAGTCAGCAACCATAGTCCCGCCCCTAACT |
| | NO: 3 | CCGCCCATCCCGCCCCTAACTCCGCCCAGTTCCGCCCATTCT |
| | | CCGCCCCATCGCTGACTAATTTTTTTTATTTATGCAGAGGCC |
| | | GAGGCCGCCTCGGCCTCTGAGCTATTCCAGAAGTAGTGAGG |
| | | AGGCTTTTTTGGAGGCCTAGGCTTTTGCAAAAAGCTT |
| miniTK | SEQ ID | TTCGCATATTAAGGTGACGCGTGTGGCCTCGAACACCGAGC |
| | NO: 4 | GACCCTGCAGCGACCCGCTTAA |
| MLP | SEQ ID | GGGGGGCTATAAAAGGGGGTGGGGGCGTTCGTCCTCACTCT |
| | NO: 5 | |
| pJB42CA | SEQ ID | CTGACAAATTCAGTATAAAAGCTTGGGGCTGGGGCCGAGCA |
| T5 | NO: 6 | CTGGGGACTTTGAGGGTGGCCAGGCCAGCGTAGGAGGCCA |
| | | GCGTAGGATCCTGCTGGGAGCGGGGAACTGAGGGAAGCGA |
| | | CGCCGAGAAAGCAGGCGTACCACGGAGGGAGAGAAAAGCT |
| | | CCGGAAGCCCAGCAGCG |
| pGL4min | SEQ ID | AGACACTAGAGGGTATATAATGGAAGCTCGACTTCCAGCTT |
| P | NO: 7 | GGCAAT |
| YB TAT | SEQ ID | TCTAGAGGGTTTATAATGGGGGCCACTAGTCTACTACTCAG |
| Ainr11 | NO: 8 | AAA |
| TCT | SEQ ID | TCTTTCTTTTCGGTTGTCAAAATTTCGAGCGGAGCGGTCGC |
| | NO: 9 | |
| YB TAT | SEQ ID | TCTAGAGGGTAGATAATGGGGGCCACTAGTCTACTACGAGA |
| Ainr9 | NO: 10 | AAG |
| YB TAT | SEQ ID | TCTAGAGGGTATATAATAATAACCACTAGTCTACTACCAGA |
| Ainr12 | NO: 11 | AAG |
| YB TAT | SEQ ID | TCTAGAGGGTATATAATGGGGGCCA |
| A | NO: 12 | |
| YB TAT | SEQ ID | TCTAGAGGGTATAAAAGGCGGGCCACTAGTCTACTATGTGG |
| Ainr7 | NO: 13 | AAG |
| YB TAT | SEQ ID | TCTAGAGGGTATATAATGGGGGCCACTAGTCTACTACGGGA |
| Ainr8 | NO: 14 | AAG |
| YB TAT | SEQ ID | TCTAGAGGGTATATAATGGGGGCCACTAGTCTAACTATCAG |
| Ainr4 | NO: 15 | TCAG |
| YB TAT | SEQ ID | TCTAGAGGGTATATAATGGGGGCCACTAGTCTACTATCAGT |
| Ainr3 | NO: 16 | CAG |
| YB TAT | SEQ ID | TCTAGAGGGTATAAAAGGCGGGCCACTAGTCTACTACCAGA |
| Ainr6 | NO: 17 | AAG |
| YB TAT | SEQ ID | TCTAGAGGGTATATAATGGGGGCCAAAGACTAGTCTACTAC |
| Ainr2 | NO: 18 | CAG |
| YB TAT | SEQ ID | CTAGAGGGTATATAATGGGGGCCACTAGTCTACTCCGTCAG |
| Ainr13 | NO: 19 | ATC |
| YB TAT | SEQ ID | TCTAGAGGGTATAAAAGGCGGGCCACTAGTCTACTATCAGT |
| Ainr5 | NO: 20 | CAG |
| YB TAT | SEQ ID | TCTAGAGGGTATATAATGGGGGCCACTAGTCTACTACCAGA |
| Ainr | NO: 21 | AAG |
| YB TAT | SEQ ID | TCTAGAGGGCTTAAAATGGGGGCCACTAGTCTACTACCAGA |
| Ainr10 | NO: 22 | AAG |
| minCMV | SEQ ID | GTAGGCGTGTACGGTGGGAGGTCTATATAAGCAGAGCTCGT |
| | NO: 23 | TTAGTGAACCGTCAGATC |
| minP | SEQ ID | ACTAGAGGGTATATAATGGAAGCTCGACTTCCAGCTTGGCA |
| | NO: 24 | ATCCGGTACTGTTATGGC |
| YB TAT | SEQ ID | ATGCTCTAGAGGGTATATAATGGGGGCCACTAGTCTACTAC |
| Afull | NO: 25 | CAGAAAGCTTGGTACCGA |
| YB TAT | SEQ ID | CATATGCTCTAGAGGGTATATAATGGGGGCCACTAGTCTAC |
| Afull | NO: 42 | TACCAGAAAGCTTGGTACCGAGCTCGGATCCAGCCA |
| minP | SEQ ID | GAGATCCAGTTTGGACACTAGAGGGTATATAATGGAAGCTC |
| | NO: 43 | GACTTCCAGCTTGGCAATCCGGTACTGTTATGGCCCAGTCC |
| | | AA |

In some embodiments, the core promoter may be cell type and/or cell state generic. A cell type and/or cell state generic core promoter may have low basal activity alone (e.g., low levels of transcriptional activation in the absence of a transcription factor binding sequence) and high activity (e.g., high levels of transcriptional activation) when paired with a transcription factor binding sequence in the presence of cell type and/or cell state specific transcription factors. For example, a cell type generic core promoter may have low transcriptional activation in the absence of a transcription factor binding sequence, independent of cell type and/or cell state. The cell type and/or cell state generic core promoter may have high transcriptional activation when paired with a cell state-specific transcription factor binding sequence in a cell type and/or cell state of interest (e.g., in the presence of, or at high levels of, transcription factors that bind to the transcription factor binding sequence). A cell type generic core promoter may have low transcriptional activation when paired with a cell type-specific transcription factor binding sequence not in a cell type of interest (e.g., in the absence of, or at low levels of, transcription factors that bind to the transcription factor binding sequence). For example, a cell type generic core promoter paired with a cell type-specific transcription factor binding sequence may be inactive in the absence of cell type-specific transcription factors and may be active in the presence of cell type-specific transcription factors. A cell state generic core promoter may have low transcriptional activation when paired with a cell state-specific transcription factor binding sequence not in a cell state of interest (e.g., in the absence of, or at low levels of, transcription factors that bind to the transcription factor binding sequence). For example, a cell state generic core promoter paired with a cell state-specific transcription factor binding sequence may be inactive in the absence of cell state-specific transcription factors (e.g., cell genotype-specific transcription factors and/or cell phenotype-specific transcription factors) and may be active in the presence of cell state-specific transcription factors. In some embodiments, a core promoter sequence may be engineered to have low basal transcriptional activation and high transcriptional activation when paired with a cell state- and/or cell type-specific transcription factor binding sequence in a cell state and/or cell type of interest.

The sequence of the core promoter may be varied to tune the transcription level, cell type specificity, cell genotype specificity, or cell phenotype specificity. In some embodiments, a core promoter sequence may comprise an endogenous core promoter sequence (e.g., TATA, CMV, EF1a, CAG, PGK, TRE, U6, or UAS). In some embodiments, a core promoter sequence may comprise a variant core promoter sequence. In some embodiments, a variant core promoter sequence may comprise at least about 40%, at least about 50%, at least about 60%, at least about 70%, at least about 80%, at least about 90%, at least about 95%, or at least about 98% sequence identity to an endogenous core promoter sequence. In some embodiments, a variant core promoter sequence may comprise no more than about 40%, no more than about 50%, no more than about 60%, no more than about 70%, no more than about 80%, no more than about 90%, no more than about 95%, no more than about 98% sequence identity to endogenous core promoter sequence. In some embodiments, a core promoter may comprise a synthetic core promoter (e.g., minimal CMV, minimal SV40, or YB_TATA). In some embodiments, the core promoter sequence may comprise at least about 40%, at least about 50%, at least about 60%, at least about 70%, at least about 80%, at least about 90%, at least about 95%, or at least about 98% sequence identity to a synthetic core promoter sequence. In some embodiments, a core promoter may comprise a core promoter sequence provided in TABLE 4. In some embodiments, the core promoter sequence may comprise at least about 40%, at least about 50%, at least about 60%, at least about 70%, at least about 80%, at least about 90%, at least about 95%, or at least about 98% sequence identity to a core promoter sequence provided in TABLE 4. In some embodiments, the core promoter sequence may comprise at least about 40%, at least about 50%, at least about 60%, at least about 70%, at least about 80%, at least about 90%, at least about 95%, or at least about 98% sequence identity to any one of SEQ ID NO: 8, SEQ ID NO: 10-SEQ ID NO: 22, SEQ ID NO: 25, or SEQ ID NO: 42. In some embodiments, the core promoter sequence may comprise any one of SEQ ID NO: 8, SEQ ID NO: 10-SEQ ID NO: 22, SEQ ID NO: 25, or SEQ ID NO: 42. In some embodiments, the core promoter sequence may consist of any one of SEQ ID NO: 8, SEQ ID NO: 10-SEQ ID NO: 22, SEQ ID NO: 25, or SEQ ID NO: 42.

A workflow for tuning the transcription level and cell state specificity (e.g., cell genotype specificity or cell phenotype specificity) of a core promoter sequence may comprise generating candidate core promoter sequences comprising variants of core promoter sequences that facilitate transcription initiation, and screening the candidate core promoter sequences for transcription level and cell state specificity. In some embodiments, a library of polynucleotides comprising different core promoter sequences may be screened for transcription level and cell state specificity. In some embodiments, core promoter sequences may be screened in combination with transcription factor binding sequences for tuning the transcription level and cell state specificity of the promoter.

A workflow for tuning the transcription level and cell type specificity (e.g., neuron specificity, hepatocyte, or muscle cell specificity) of a core promoter sequence may comprise generating candidate core promoter sequences comprising variants of core promoter sequences that facilitate transcription initiation, and screening the candidate core promoter sequences for transcription level and cell type specificity. In some embodiments, a library of polynucleotides comprising different core promoter sequences may be screened for transcription level and cell type specificity. In some embodiments, core promoter sequences may be screened in combination with transcription factor binding sequences for tuning the transcription level and cell type specificity of the promoter.

A workflow for tuning the transcription level and cell state and cell type specificity of a core promoter sequence may comprise generating candidate core promoter sequences comprising variants of core promoter sequences that facilitate transcription initiation, and screening the candidate core promoter sequences for transcription level, cell state specificity, and cell type specificity. In some embodiments, a library of polynucleotides comprising different core promoter sequences may be screened for transcription level, cell state specificity, and cell type specificity. In some embodiments, core promoter sequences may be screened in combination with transcription factor binding sequences for tuning the transcription level, cell state specificity, and cell type specificity of the promoter.

### Promoter Constructs

The transcription factor binding polynucleotide and the core promoter described herein may be combined to generate a promoter construct. The transcription factor binding sequences and the core promoter sequences described herein may be combined to generate a sequence of a promoter construct. The core promoter may recruit transcriptional machinery to initiate transcription of the sequence downstream of the core promoter when in combination with a transcription factor binding sequence that is bound to cognate ligands, coactivators, or corepressors. The promoter construct may be engineered to bind cell type- and/or cell state-specific transcription factors via the transcription factor binding sequence and initiate transcription by binding of transcriptional machinery to the core promoter sequence. In some embodiments, a promoter construct may comprise a transcription factor binding sequence (e.g., a transcription factor binding sequence provided in TABLE 3) or one or more transcription factor binding motifs (e.g., a transcription factor binding motif provided in TABLE 2 or that binds to a transcription factor provided in TABLE 1) and a core promoter sequence (e.g., a core promoter sequence provided in TABLE 4).

Examples of promoter constructs that promote cell type- and/or cell state-specific transcription are provided in TABLE 5. The promoters provided in TABLE 5 contain a transcription factor binding sequence of SEQ ID NO: 26. However, the transcription factor binding motif of any of the promoter constructs provided in TABLE 5 may be replaced with any of the transcription factor binding sequences provided in TABLE 3 or with one or more of the transcription factor binding motifs provided in TABLE 2. In some embodiments, a promoter sequence may comprise at least about 40%, at least about 50%, at least about 60%, at least about 70%, at least about 80%, at least about 85%, at least about 87%, at least about 90%, at least about 93%, at least about 95%, or at least about 98%, or about 100% sequence identity to a promoter sequence provided in TABLE 5.

TABLE 5
Exemplary Promoter Constructs and Corresponding Cell State or Cell Type

| | | Cell |
|---|---|---|
| SEQ ID NO | Sequence | Type/State |
| SEQ ID | AAGGTAGCTTCCAACCGACGGACGGGGCAGCGGGTAC | MeCP2 |
| NO: 113 | CCGCTGGGGCAGTGCCGCAGCGGCAGGAATACGGAGG | mutant |
| | GCAGCGGGTACCCGCTGGGGCAGTGCCGCAGCGGCAG | neurons |
| | GAATACGGAGCAGACACTAGAGGGTATATAATGGAAG | |
| | CTCGACTTCCAGCTTGGCAATC | |
| SEQ ID | AAGGTAGCTTCCAGTAACCGACGGACGGGGCAGCGGG | MeCP2 |
| NO: 114 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTTTATAATGGGGGC | |
| | CACTAGTCTACTACTCAGAAAC | |
| SEQ ID | AAGGTAGCTTCCAGTACGCACCGACGGACGGGGCAGC | MeCP2 |
| NO: 115 | GGGTACCCGCTGGGGCAGTGCCGCAGCGGCAGGAATA | mutant |
| | CGGAGGGCAGCGGGTACCCGCTGGGGCAGTGCCGCAG | neurons |
| | CGGCAGGAATACGGAGTTCTTTCTTTTCGGTTGTCAAA | |
| | ATTTCGAGCGGAGCGGTCGCC | |
| SEQ ID | AAGGTAGCTTCCAGTAACCGACGGACGGGGCAGCGGG | MeCP2 |
| NO: 116 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTAGATAATGGGGGC | |
| | CACTAGTCTACTACGAGAAAGC | |
| SEQ ID | AAGGTAGCTTCCAGTAACCGACGGACGGGGCAGCGGG | MeCP2 |
| NO: 117 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATATAATAATAAC | |
| | CACTAGTCTACTACCAGAAAGC | |
| SEQ ID | AAGGTAGCTTCCAGTACGCCTCGTTACTTCGGAGTACC | MeCP2 |
| NO: 118 | GACGGACGGGGCAGCGGGTACCCGCTGGGGCAGTGCC | mutant |
| | GCAGCGGCAGGAATACGGAGGGCAGCGGGTACCCGCT | neurons |
| | GGGGCAGTGCCGCAGCGGCAGGAATACGGAGTTCTAG | |
| | AGGGTATATAATGGGGGCCAC | |
| SEQ ID | AAGGTAGCTTCCAGTAACCGACGGACGGGGCAGCGGG | MeCP2 |
| NO: 119 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATAAAAGGCGGGC | |
| | CACTAGTCTACTATGTGGAAGC | |
| SEQ ID | AAGGTAGCTTCCAGTAACCGACGGACGGGGCAGCGGG | MeCP2 |
| NO: 120 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATATAATGGGGGC | |
| | CACTAGTCTACTACGGGAAAGC | |
| SEQ ID | AAGGTAGCTTCCAGTACCGACGGACGGGGCAGCGGGT | MeCP2 |
| NO: 121 | ACCCGCTGGGGCAGTGCCGCAGCGGCAGGAATACGGA | mutant |
| | GGGCAGCGGGTACCCGCTGGGGCAGTGCCGCAGCGGC | neurons |
| | AGGAATACGGAGATCTAGAGGGTATATAATGGGGGCC | |
| | ACTAGTCTAACTATCAGTCAGC | |
| SEQ ID | AAGGTAGCTTCCAGTAACCGACGGACGGGGCAGCGGG | MeCP2 |
| NO: 122 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATATAATGGGGGC | |
| | CACTAGTCTACTATCAGTCAGC | |
| SEQ ID | AAGGTAGCTTCCAGTAACCGACGGACGGGGCAGCGGG | MeCP2 |
| NO: 123 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATAAAAGGCGGGC | |
| | CACTAGTCTACTACCAGAAAGC | |
| SEQ ID | AAGGTAGCTTCCAGTAACCGACGGACGGGGCAGCGGG | MeCP2 |
| NO: 124 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATATAATGGGGGC | |
| | CAAAGACTAGTCTACTACCAGC | |
| SEQ ID | AAGGTAGCTTCCAGTAACCGACGGACGGGGCAGCGGG | MeCP2 |
| NO: 125 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTCTAGAGGGTATATAATGGGGGCC | |
| | ACTAGTCTACTCCGTCAGATCC | |
| SEQ ID | AAGGTAGCTTCCAGTAACCGACGGACGGGGCAGCGGG | MeCP2 |
| NO: 126 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATAAAAGGCGGGC | |
| | CACTAGTCTACTATCAGTCAGC | |
| SEQ ID | AAGGTAGCTTCCAGTAACCGACGGACGGGGCAGCGGG | MeCP2 |
| NO: 127 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGTATATAATGGGGGC | |
| | CACTAGTCTACTACCAGAAAGC | |
| SEQ ID | AAGGTAGCTTCCAGTAACCGACGGACGGGGCAGCGGG | MeCP2 |
| NO: 128 | TACCCGCTGGGGCAGTGCCGCAGCGGCAGGAATACGG | mutant |
| | AGGGCAGCGGGTACCCGCTGGGGCAGTGCCGCAGCGG | neurons |
| | CAGGAATACGGAGTTCTAGAGGGCTTAAAATGGGGGC | |
| | CACTAGTCTACTACCAGAAAGC | |
| SEQ ID | AACCGACGGACGGGGCAGCGGGTACCCGCTGGGGCAG | MeCP2 |
| NO: 129 | TGCCGCAGCGGCAGGAATACGGAGGGCAGCGGGTACC | mutant |
| | CGCTGGGGCAGTGCCGCAGCGGCAGGAATACGGAGGG | neurons |
| | TAGGCGTGTACGGTGGGAGGTCTATATAAGCAGAGCT | |
| | CGTTTAGTGAACCGTCAGATCC | |
| SEQ ID | AACCGACGGACGGGGCAGCGGGTACCCGCTGGGGCAG | MeCP2 |
| NO: 130 | TGCCGCAGCGGCAGGAATACGGAGGGCAGCGGGTACC | mutant |
| | CGCTGGGGCAGTGCCGCAGCGGCAGGAATACGGAGGA | neurons |
| | CTAGAGGGTATATAATGGAAGCTCGACTTCCAGCTTGG | |
| | CAATCCGGTACTGTTATGGCC | |
| SEQ ID | AACCGACGGACGGGGCAGCGGGTACCCGCTGGGGCAG | MeCP2 |
| NO: 131 | TGCCGCAGCGGCAGGAATACGGAGGGCAGCGGGTACC | mutant |
| | CGCTGGGGCAGTGCCGCAGCGGCAGGAATACGGAGGA | neurons |
| | TGCTCTAGAGGGTATATAATGGGGGCCACTAGTCTACT | |
| | ACCAGAAAGCTTGGTACCGAC | |
| SEQ ID | AGGACCGGATCAACTAAGGTAGCTTCCAGTACGCCTCG | MeCP2 |
| NO: 132 | TTACTTCGGAGTTACGTATACTCACGCGTAAGTTGCCG | mutant |
| | AATAGGTGCACTATGACTGGAGTGCTTAGCGCGTGATT | neurons |
| | ACTGATGGACACGTCACCTTGGCGATTCTAGAGGGTAT | |
| | ATAATGGGGGCCACTAGTCTACTACCAGAAAGCTTGGT | |
| | ACCGAGCTCG | |
| SEQ ID | AGGACCGGATCAACTAGAAGAACAACCGTACGCCACT | MeCP2 |
| NO: 133 | AACGATCGAAGCTTGATCAATTGAAGAATAATAGTGG | mutant |
| | ACCAGCCGGTATCCACAGTCTCAAGACCCTTTCCAAGA | neurons |
| | AGGTATCTGCTTTCCAGGAAGGACCTACTCTAGAGGGT | |
| | ATATAATGGGGGCCACTAGTCTACTACCAGAAAGCTTG | |
| | GTACCGAGCTCG | |
| SEQ ID | AGGACCGGATCAACTAAGGTAGCTTCCAGTACGCCTCG | MeCP2 |
| NO: 134 | TTACTTCGGAGTTACGTATACTCACGCGTAAGTTGCCG | mutant |
| | AATAGGTGTGGCACGTGCCAAGTGCTCGGCACGTGCC | neurons |
| | GACTGCTCGGCACGTGCCATTGGCGATTCTAGAGGGTA | |
| | TATAATGGGGGCCACTAGTCTACTACCAGAAAGCTTGG | |
| | TACCGAGCTCG | |
| SEQ ID | AGGACCGGATCAACTAAGGTAGCTTCCAGTACGCCTCG | MeCP2 |
| NO: 135 | AGGGAAGTGGGTCAGAAGGACTCACGGAAATTTGGGA | mutant |
| | CAGAAGATGCACTAAGCAAGTGGGTCAGAAGGCGTGA | neurons |
| | TAGGGAAGTGGGTCAGAAGGTTGGCGATTCTAGAGGG | |
| | TATATAATGGGGGCCACTAGTCTACTACCAGAAAGCTT | |
| | GGTACCGAGCTCG | |
| SEQ ID | AGGACCGGATCAACTAAGGTAGCTTCCAGTACGCCTCG | MeCP2 |
| NO: 136 | TTACTTCGGAGTTACGTATACTCACCCTTTGATGTGGG | mutant |
| | AATAGTCCTTTGATGACGGGAGTGCCCTTTGATGTTGT | neurons |
| | ACTGCCCCTTTGATGTGGTTGGCGATTCTAGAGGGTAT | |
| | ATAATGGGGGCCACTAGTCTACTACCAGAAAGCTTGGT | |
| | ACCGAGCTCG | |
| SEQ ID | AGGACCGGATCAACTAAGGTAGCTTCCAGTACGCCTCG | MeCP2 |
| NO: 137 | TTACTTCGGAGTTACGTATACTCATCAAGGTCATGCCG | mutant |
| | ACTTGGTTCCGTTCCACTGGAGTGTCAAGGTCATGATT | neurons |
| | ACTTGGTTCCGTTCCACTTTGGCGATTCTAGAGGGTAT | |
| | ATAATGGGGGCCACTAGTCTACTACCAGAAAGCTTGGT | |
| | ACCGAGCTCG | |
| SEQ ID | AGGACCGGATCAACTAAGGTAGCTTCCAGTACGCCTCG | MeCP2 |
| NO: 138 | TTACTTCGGAGTGGAAATTTGGGACAGAAGAGTTGCCT | mutant |
| | CCTTTGATGACGATGACTGGAAATTTGGGACAGAAGAT | neurons |
| | ACTGCTCCTTTGATGACGTTGGCGATTCTAGAGGGTAT | |
| | ATAATGGGGGCCACTAGTCTACTACCAGAAAGCTTGGT | |
| | ACCGAGCTCG | |
| SEQ ID | AGGACCGGATCAACTAGAAGAACAACCGTACGCCACT | MeCP2 |
| NO: 139 | AACGATCGAAGCTTGATCAATTGAAGAATAATAGTGG | mutant |
| | ACCAGCCGGTATCTCAAGCTCGAAGAGACACAGATGG | neurons |
| | CCGGTAAACAATTGCATTGTTGGACCTACTCTAGAGGG | |
| | TATATAATGGGGGCCACTAGTCTACTACCAGAAAGCTT | |
| | GGTACCGAGCTCG | |
| SEQ ID | AGGACCGGATCAACTAGAAGAACAACCGTACGCCACT | MeCP2 |
| NO: 140 | AACGATCGAAGCTTGATCAATTGAAGAATAATAGTGG | mutant |
| | ACCAGCCGGTATCCACAGTCTCAAGAGAGAGGACAGG | neurons |
| | CCGGTATCGACTCAAGCGACAGGACCTACTTATCTTTC | |
| | TTTTCGGTTGTCAAAATTTCGAGCGGAGCGGTCGCCTT | |
| | GGTACCGAGCTCG | |

In some embodiments, a promoter may comprise a core promoter sequence and a transcription factor binding sequence having at least 90% sequence identity to SEQ ID NO: 26. In some embodiments, a promoter may comprise a core promoter sequence and a transcription factor binding sequence having at least 93% sequence identity to SEQ ID NO: 26. In some embodiments, a promoter may comprise a core promoter sequence and a transcription factor binding sequence having at least 95% sequence identity to SEQ ID NO: 26. In some embodiments, a promoter may comprise a core promoter sequence and a transcription factor binding sequence having at least 98% sequence identity to SEQ ID NO: 26. In some embodiments, a promoter may comprise a core promoter sequence and a transcription factor binding sequence that is SEQ ID NO: 26. In some embodiments, the core promoter is any core promoter provided in TABLE 4. In some embodiments, the core promoter sequence may comprise at least about 40%, at least about 50%, at least about 60%, at least about 70%, at least about 80%, at least about 90%, at least about 95%, or at least about 98% sequence identity to a core promoter sequence provided in TABLE 4.

A promoter may be combined with a payload sequence to form a polynucleotide construct that promotes cell type- and/or cell state-specific transcription of the payload sequence. In some embodiments, the payload sequence is transcribed in a target cell (e.g., a target cell type or a target cell state) at a level at is at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 10-fold, at least about 20-fold, at least about 30-fold, at least about 40-fold, at least about 50-fold, at least about 100-fold, at least about 150-fold, or at least about 200-fold the level in a non-target cell (e.g., a non-target cell type or a non-target cell state). For example, a payload sequence may be transcribed in a MeCP2 mutant cell (e.g., a cell expressing a mutant MeCP2 protein) at a level that is at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 10-fold, at least about 20-fold, at least about 30-fold, at least about 40-fold, at least about 50-fold, at least about 100-fold, at least about 150-fold, or at least about 200-fold the level in a MeCP2 wild type cell (e.g., a cell expressing a wild type MeCP2 protein). In some embodiments, a MeCP2 mutant cell is a cell expressing a mutant MeCP2 protein. In some embodiments, a MeCP2 mutant cell is a cell expressing a mutant MeCP2 protein associated with disease phenotype, such as Rett syndrome. In some embodiments, a MeCP2 mutant cell is a diseased cell having a diseased phenotype associated with the expression of a protein from a MECP2 mutant gene and comprising the MECP2 mutant gene. In some embodiments, a MeCP2 wild type cell is a cell expressing a wild type MeCP2 protein. In some embodiments, a MeCP2 wild type cell is a cell expressing a wild type MeCP2 protein associated with wild type phenotype. In some embodiments, a MeCP2 wild type cell is a healthy cell having a wild type phenotype associated with the expression of a protein from a wild type MECP2 gene and comprising the wild type MECP2 gene.

### Payloads

A payload of the present disclosure may comprise a sequence encoding a protein under transcriptional control of a promoter (e.g., a promoter comprising a transcription factor binding polynucleotide and a core promoter). The payload may comprise a transgene for delivery to a cell (e.g., a cell of a human or non-human subject). In some embodiments, the transgene may comprise a coding sequence encoding a protein (e.g., a protein without a mutation associated with a disease or condition). Upon delivery of the payload to a cell, the protein encoded by the coding sequence may be expressed in the cell. In some embodiments, expression of a protein encoded by the coding sequence may treat, prevent, or alleviate symptoms of a disease or disorder. In some embodiments, the transgene may encode a wild type copy of a protein that is mutated or dysregulated in the disease or condition. Alternatively or in addition, the payload sequence may encode a therapeutic polynucleotide (e.g., a gRNA or tRNA) for delivery to a cell (e.g., a cell of a human or non-human subject). In some embodiments, the therapeutic polynucleotide may target a gene (e.g., for gene editing). Upon delivery of the payload to a cell, the therapeutic polynucleotide encoded by the payload sequence may be expressed in the cell. In some embodiments, expression of the therapeutic polynucleotide may treat, prevent, or alleviate symptoms of a disease or disorder. In some embodiments, the therapeutic polynucleotide may target a mutated gene sequence associated with the disease or disorder.

In some embodiments, cell state specific transcription of a payload sequence (e.g., a transgene or therapeutic polynucleotide) is desired. For example, a transgene lacking a mutation may be specifically transcribed in neurons having a gene comprising the mutation or having a phenotype associated with the mutation. In another example, a transgene lacking a mutation may be specifically transcribed in retinal tissue having gene comprising the mutation or having a phenotype associated with the mutation. In another example, a transgene lacking a genetic variation may be specifically transcribed in cells having the genetic variation or having a phenotype associated with the genetic variation. In another example, a transgene encoding a protein or polynucleotide may be specifically transcribed in cells having altered expression (e.g., elevated expression or decreased expression) of the protein or polynucleotide. Alternatively or in addition, a therapeutic polynucleotide targeting a mutated gene sequence may be specifically transcribed in neurons having a gene comprising the mutation or having a phenotype associated with the mutation. In another example, a therapeutic polynucleotide targeting a mutated gene sequence may be specifically transcribed in retinal tissue having gene comprising the mutation or having a phenotype associated with the mutation. In another example, a therapeutic polynucleotide targeting a mutated gene sequence may be specifically transcribed in cells having the genetic variation or having a phenotype associated with the genetic variation. In another example, a therapeutic polynucleotide targeting a gene sequence may be specifically transcribed in cells having altered expression of a protein or polynucleotide encoded by the gene sequence.

Examples of genes that may be encoded in the payload sequence (e.g., a transgene) or may be targeted by a therapeutic polynucleotide encoded by the payload sequence (e.g., a gRNA or tRNA) are provided in TABLE 6. In some embodiments, the genes may be delivered as transgenes to a cell of a subject to treat a disease or condition in the subject. In some embodiments, the transgene may encode a wild type copy of a gene provided in TABLE 6. Alternatively or in addition, a therapeutic polynucleotide encoded by the payload sequence may target a mutated version of a gene provided in TABLE 6.

TABLE 6
Exemplary Transgene Payloads or Gene Targets and Indications

| Transgene or | |
|---|---|
| Gene Target | Indication |
| MECP2 | Rett syndrome; MECP2 duplication syndrome |
| GRN | Frontotemporal dementia; neuronal ceroid |
| | lipofuscinosis |
| PRPH2 | Retinitis Pigmentosa 7; macular degeneration |
| RHO | Retinitis Pigmentosa 4 |
| UBE3A | Angelman Syndrome |
| DYRK1A | DYRK1A haploinsufficiency |
| MEF2C | MEF2C haploinsufficiency syndrome |
| NSD1 | Sotos syndrome; Reverse Sotos syndrome |
| ATRX | Alpha-thalassemia X-linked intellectual |
| | disability syndrome |
| RPS6KA3 | Xp22.12 duplication; Coffin-Lowry syndrome |
| TCF4 | Pitt Hopkins syndrome |
| ZEB2 | Mowat-Wilson Syndrome |
| FOXG1 | FOXG1 syndrome |
| CDKL5 | CDKL5 deficiency disorder; West Syndrome |
| Partial piece | 2q23.1 microdeletion syndrome |
| of Chromo- | |
| some 2 | |
| SLC6A1 | Doose Syndrome; SLC6A1 epileptic encephalopathy |
| DMD | Duchenne's muscular dystrophy; Becker |
| | muscular dystrophy |
| SERPINA1 | Alpha-1 antitrypsin deficiency (AATD) |
| ABCA4 | Macular Degeneration/Stargardt disease |
| CFTR | Cystic Fibrosis |
| HEXA | Tay-Sachs |
| RAB7A | Charcot-Marie-Tooth neuropathy |
| ATP7B | Wilson's disease |
| HFE | Hereditary Hemochromatosis |
| LIPA | Wolman disease; cholesteryl ester storage disease |
| SCNN1A | Psueodhypoaldosteronism type 1 |
| PKD1 | Polycystic Kidney, Autosomal Dominant |
| PKD2 | Polycystic Kidney, Autosomal Dominant |
| PKHD1 | Autosomal Recessive Polycystic Kidney Disease |
| ACE | Kidney Failure, Chronic |
| ALB | Kidney Failure, Chronic |
| VHL | Malignant neoplasm of kidney |
| EPO | Kidney Failure, Chronic |
| PKD2 | Polycystic kidney disease, type 2 |
| FH | Malignant neoplasm of kidney |
| ACE | Kidney Failure |
| TNF | Kidney Failure, Chronic |
| SPP1 | Kidney Calculi |
| IL6 | Kidney Failure, Chronic |
| MYH9 | Kidney Failure, Chronic |
| PKD1 | Autosomal Recessive Polycystic Kidney Disease |
| TSC2 | Kidney Neoplasm |
| ADIPOQ | Kidney Failure, Chronic |
| IL2 | Malignant neoplasm of kidney |
| CCL2 | Kidney Failure, Chronic |
| TGFB1 | Kidney Failure, Chronic |
| VHL | Kidney Neoplasm |
| UMOD | Medullary Cystic Kidney Disease Type 2 |
| BCOR | Clear cell sarcoma of kidney |
| FLCN | Kidney Neoplasm |
| FLCN | Malignant neoplasm of kidney |
| PKD1 | Polycystic kidney disease, type 2 |
| TP53 | Malignant neoplasm of kidney |
| CRP | Kidney Failure, Chronic |
| PTEN | Malignant neoplasm of kidney |
| IFT88 | Autosomal Recessive Polycystic Kidney Disease |
| CLDN14 | Kidney Calculi |
| FH | Kidney Neoplasm |
| VHL | Collecting Duct Carcinoma of the Kidney |
| AGT | Kidney Failure |
| MET | Malignant neoplasm of kidney |
| MYH9 | Kidney Failure |
| YWHAE | Clear cell sarcoma of kidney |
| PKD1 | Polycystic Kidney - body part |
| HAMP | Kidney Failure, Chronic |
| EPO | Kidney Failure |
| MUC1 | Medullary cystic kidney disease 1 |
| BAP1 | Malignant neoplasm of kidney |
| APOE | Kidney Failure |
| CYBA | Kidney Failure, Chronic |
| GSTT1 | Malignant neoplasm of kidney |
| IFNG | Kidney Failure |
| IGF1 | Kidney Failure, Chronic |
| IL2 | Kidney Neoplasm |
| ABCB1 | Malignant neoplasm of kidney |
| SDHB | Malignant neoplasm of kidney |
| TP53 | Collecting Duct Carcinoma of the Kidney |
| TSC2 | Malignant neoplasm of kidney |
| BRAF | Kidney Neoplasm |
| CDKN1B | Malignant neoplasm of kidney |
| GLA | Kidney Failure; Chronic kidney disease/disorder with a |
| | monogenetic origin |
| KRT7 | Collecting Duct Carcinoma of the Kidney |
| PPARG | Polycystic Kidney, Autosomal Dominant |
| RET | Congenital absence of kidneys syndrome |
| TRPC6 | Kidney Failure, Chronic kidney disease/disorder with a |
| | monogenetic origin |
| NDRG1 | Malignant neoplasm of kidney |
| GANAB | Polycystic Kidney, Autosomal Dominant |
| NOX4 | Kidney Failure, Chronic |
| ADIPOR1 | Kidney Failure, Chronic |
| GREB1L | Congenital absence of kidneys syndrome |
| ANKS6 | Cystic kidney |
| NUTM2B | Clear cell sarcoma of kidney |
| CAT | Kidney Failure, Chronic |
| CYBA | Kidney Failure |
| CYBB | Kidney Failure, Chronic |
| EGFR | Autosomal Recessive Polycystic Kidney Disease |
| HMOX1 | Kidney Failure, Chronic |
| LRP2 | Kidney Failure, Chronic |
| SERPINE1 | Kidney Failure, Chronic |
| PAX2 | Kidney Failure, Chronic; Chronic kidney disease/disorder |
| | with a monogenetic origin |
| ABCB1 | Kidney Neoplasm |
| PPARA | Kidney Failure, Chronic |
| PPARG | Kidney Failure |
| PTGS2 | Kidney Failure, Chronic |
| RELA | Kidney Failure, Chronic |
| RET | Unilateral agenesis of kidney |
| TLR4 | Kidney Failure |
| UMOD | Glomerulocystic Kidney Disease with Hyperuricemia and |
| | Isosthenuria |
| BAP1 | Kidney Neoplasm |
| RETN | Kidney Failure, Chronic |
| GREB1L | Unilateral agenesis of kidney |
| FRAS1 | Unilateral agenesis of kidney |
| CRB2 | Cystic Kidney Disease with Ventriculomegaly |
| APRT | Kidney Failure, Chronic |
| AXL | Malignant neoplasm of kidney |
| CCND1 | Malignant neoplasm of kidney |
| BRAF | Malignant neoplasm of kidney |
| CBR1 | Kidney Failure, Chronic |
| CPT1A | Kidney Failure, Chronic |
| CYP1A1 | Malignant neoplasm of kidney |
| CYP2B6 | Kidney Failure, Chronic |
| EDN1 | Kidney Failure |
| ERBB2 | Collecting Duct Carcinoma of the Kidney |
| HMGCR | Kidney Failure, Chronic |
| MME | Kidney Failure |
| NFKB1 | Kidney Failure, Chronic |
| NGF | Kidney Failure, Chronic |
| MAPK1 | Malignant neoplasm of kidney |
| MAPK3 | Malignant neoplasm of kidney |
| PTGS2 | Kidney Neoplasm |
| PTGS2 | Malignant neoplasm of kidney |
| HLTF | Kidney Neoplasm |
| SOD1 | Kidney Calculi |
| SOD2 | Malignant neoplasm of kidney |
| SREBF2 | Kidney Failure, Chronic |
| HNF1B | Unilateral Multicystic Dysplastic Kidney |
| TERT | Clear cell sarcoma of kidney |
| TNFSF10 | Malignant neoplasm of kidney |
| NDRG1 | Kidney Neoplasm |
| MBTPS2 | Brain Anomalies, Retardation, Ectodermal Dysplasia, |
| | Skeletal Malformations, Hirschsprung Disease, Ear-Eye |
| | Anomalies, Cleft Palate-Cryptorchidism, And Kidney |
| | Dysplasia-Hypoplasia |
| WNT4 | Sex Reversal, Female, With Dysgenesis Of Kidneys, |
| | Adrenals, And Lungs |
| BCOR | Kidney Neoplasm |
| INF2 | Kidney Failure; Chronic kidney disease/disorder with a |
| | monogenetic origin |
| ALG9 | Polycystic Kidney Disease, Potter Type I, with |
| | Microbrachycephaly, Hypertelorism, and Brachymelia |
| BICC1 | Polycystic Kidney, Autosomal Dominant |
| TMEM67 | Cystic kidney |
| IRX2 | Clear cell sarcoma of kidney |
| FREM1 | Congenital absence of kidneys syndrome |
| ANKS6 | Polycystic Kidney, Autosomal Dominant |
| FREM2 | Unilateral agenesis of kidney |
| CD46 | Chronic kidney disease/disorder with a monogenetic origin |
| COL4A3 | Chronic kidney disease/disorder with a monogenetic origin |
| COL4A4 | Chronic kidney disease/disorder with a monogenetic origin |
| COL4A5 | Chronic kidney disease/disorder with a monogenetic origin |
| TTC21B | Chronic kidney disease/disorder with a monogenetic origin |
| NPHP4 | Chronic kidney disease/disorder with a monogenetic origin |
| CD2AP | Chronic kidney disease/disorder with a monogenetic origin |
| CFI | Chronic kidney disease/disorder with a monogenetic origin |
| LAMB2 | Chronic kidney disease/disorder with a monogenetic origin |
| LMX1B | Chronic kidney disease/disorder with a monogenetic origin |
| MYH9 | Chronic kidney disease/disorder with a monogenetic origin |
| CNGA3 | Achromatopsia |
| CNGB3 | Achromatopsia |
| ABCD1 | Adrenomyeloneuropathy |
| Tafazzin | Barth Syndrome |
| CLN1 | Batten Disease |
| CLN2 | Batten Disease |
| CLN3 | Batten Disease |
| CLN4 | Batten Disease |
| CLN5 | Batten Disease |
| CLN6 | Batten Disease |
| ASPA | Canavan Disease |
| CYP21A2 | Congenital Adrenal Hyperplasia |
| PMM2 | Congenital Disorder of Glycosylation 1a |
| LAMP2 | Danon Disease |
| GLA | Fabry Disease |
| GBA | Gaucher Disease |
| GLB1 | GM1 Gangliosidosis |
| G6PC | Glycogen SD 1a |
| F9 | Hemophilia |
| Serping1 | Hereditary Angioedema |
| GALC | Krabbe Disease |
| GUCY2D | Leber's Disease |
| ND1 | Leber's Disease |
| ND6 | Leber's Disease |
| ND4 | Leber's Disease |
| RPE65 | Leber's Disease |
| AIPL1 | Leber's Disease |
| MTM1 | Myotubular Myopathy |
| MSP I | Mucopolysaccharidosis |
| MSP II | Mucopolysaccharidosis |
| MSP IIA | Mucopolysaccharidosis |
| MSP IIC | Mucopolysaccharidosis |
| MSP IIID | Mucopolysaccharidosis |
| MSP IVA | Mucopolysaccharidosis |
| MSP VI | Mucopolysaccharidosis |
| MSP VII | Mucopolysaccharidosis |
| MSP IXA | Mucopolysaccharidosis |
| OTC | Ornithine Transcarbamylase Deficiency |
| GAA | Pompe Disease |
| HEXB | Sandhoff Disease |
| SMN1 | Spinal Muscular Atrophy |
| USH2D- | Usher Syndrome |
| WHRN | |
| USH3A- | Usher Syndrome |
| CLN1 | |
| SOD1 | ALS |
| HBB | Beta-thalassemia or Sickle Cell disease |
| BEST1 | Bestrophinopathy |
| CHM | Chorioderemia |
| FXN | Freidreich's Ataxia |
| SLC37A4 | GSD1b |
| IGF1 | Osteoporosis |
| RPGR | Retinitis Pigmentosa |
| USH1C | Usher 1C, 1F |
| CIB2 | Usher 1C, 1F |
| SERPINA1 | Alpha-1 Antitrypsin Deficiency |
| AC6 | Heart Failure |
| Serca2 | Heart Failure |
| VEGF-B | Heart Failure |
| PP1 | Heart Failure |
| GAD | Parkinson's Disease |
| AADC | AADC deficiency |
| CLN2 | Batten disease (CLN2) |
| CLN6 | Batten disease (CLN6) |
| NAGLU | MPS-IIIB |
| GRN | Frontotemporal dementia with GRN mutations (FTD-GRN) |
| GBA1 | Parkinson's Disease with GBA1 mutations (PD-GBA) and |
| | neuronopathic Gaucher's disease |
| GBA1 + | Synucleinopathies |
| alpha- | |
| synuclein | |
| ASPA | Canavan disease |
| AADC | Parkinson disease |
| GDNF | Parkinson disease |
| Neurturin | Parkinson disease |
| hFOXG1 | FOXG1 syndrome |
| hKCNQ2 | KCNQ2 Encephalopathy |
| hFMR1 | Fragile X syndrome |
| anti-Tau | Alzheimer's disease |
| miRNA | |
| EPM2A or | Lafora disease |
| EPM2B | |
| SMN | SMA |
| GAN | Giant axonal neuropathy |
| CNGB3 | Achromatopsia |
| REP1 | Choroideraemia |
| RPE65 | LCA |
| ND4 | LHON |
| RLBP1 | RP (RLBP1) |
| Anti-VEGF | Wet AMD |
| antibody | |
| RPGR | X-linked RP |
| RS1 | X-linked retinoschisis |
| HEXB and | (Nervous system) |
| HEXA | |
| human | (Nervous system) |
| codon- | |
| optimized | |
| CLN1 | |
| comple- | |
| mentary | |
| DNA | |
| SURF1 | (Nervous system) |
| MECP2 | Rett Syndrome (Nervous system) |
| anti-UBE3A- | (Nervous system) |
| ATS shRNA | |
| anti-EHMT2 | (possibly germ-line cells) |
| shRNA | |
| hSLC6A1 | (Nervous system) |
| TMC1 | (Associated with hearing loss) |
| UBE3A | Angelman syndrome (nervous system) |
| AADC | AADC deficiency |
| Multiple, in- | Alzheimer's Disease |
| cluding APP, | |
| SNCA, | |
| MAPT, | |
| ApoE, NGF, | |
| TERT | |
| MAPT | Tauopathies |
| SNCA | Synucleinopathies |
| CLN2 | Batten disease (CLN2) |
| CLN3 | Batten disease (CLN3) |
| CLN6 | Batten disease (CLN6) |
| NAGLU | MPS-IIIB |
| GRN | Frontotemporal dementia with GRN mutations (FTD-GRN) |
| GBA1 | Parkinson's Disease with GBA1 mutations (PD-GBA) and |
| | neuronopathic Gaucher's disease |
| MAPT | Corticobasal Degeneration (CBD) |
| MAPT | Progressive Supranuclear Palsy (PSP) |
| MAPT | Chronic Traumatic Encephalopathy (CTE) |
| GBA1 + | Synucleinopathies |
| alpha- | |
| synuclein | |
| GBA | Gaucher disease type 2 |
| ASPA | Canavan Disease |
| AADC | Parkinson disease |
| GDNF | Parkinson disease |
| Neurturin | Parkinson disease |
| (NRTN) | |
| GAD | Parkinson disease |
| NTN | Parkinson disease |
| hFOXG1 | Parkinson disease |
| hKCNQ2 | Parkinson disease |
| hFMR1 | Parkinson disease |
| anti-Tau/ | Parkinson disease |
| miRNA | |
| EPM2A or | Parkinson disease |
| EPM2B | |
| LRRK2 | Parkinson disease |
| LRRK2 | Parkinson's Disease |
| SNCA | Parkinson's Disease |
| HEXA | Tay-Sachs Disease |
| IT15 | Huntington's disease |
| CYP46A1 | Huntington's disease |
| HTT | Huntington's disease |
| IT15 | Potocki-Lupski Syndrome |
| C9orf72 | Amyotrophic lateral sclerosis |
| SOD1 | Amyotrophic lateral sclerosis |
| DYRK1A | Down syndrome |
| SGSH | Sanfilippo disease type A |
| hNAGLU | Sanfilippo disease type B |
| HEXB and | (Nervous system) |
| HEXA | |
| human | (Nervous system) |
| codon- | |
| optimized | |
| CLN1 | |
| comple- | |
| mentary | |
| DNA | |
| SURF1 | (Nervous system) |
| anti-UBE3A- | (Nervous system) |
| ATS shRNA | |
| hSLC6A1 | (Nervous system) |
| MECP2 | Rett syndrome |
| NTF3 | Charcot-Marie-Tooth disease type 1A (CMT1A) |
| Micro- | Duchenne muscular dystrophy (DMD) |
| dystrophin | |
| Mini- | Duchenne muscular dystrophy (DMD) |
| dystrophin | |
| DUX4 | Facioscapulohumeral muscular dystrophy-1 (FSHD) |
| DYSF | Dysferlinopathy |
| GAA | Pompe disease |
| FKRP | Limb-girdle muscular dystrophies (LGMD) (2i/R9) |
| DMD | Duchenne muscular dystrophy (DMD) |
| DUX4 | Facioscapulohumeral Dystrophy |
| DMPK | Myotonic Dystrophy |
| anti-GYS1 | Glycogen storage disorders |
| miRNA | |
| MTM1 | X-linked myotubular myopathy (X-linked MTM) |
| anti-EHMT2 | euchromatic histone-lysine N-methyltransferase 2 |
| shRNA | |

Examples of genes that may be encoded by a payload sequence (e.g., the transgene) or may be targeted by a polynucleotide (e.g., a gRNA or tRNA) encoded by the payload sequence and delivered to a cell of a subject to treat, prevent, or alleviate symptoms of a disease or condition include MECP2, GRN, PRPH2, RHO, UBE3A, DYRK1A, MEF2C, NSD1, ATRX, RPS6KA3, TCF4, ZEB2, FOXG1, CDKL5, a partial piece of chromosome 2, SLC6A1, DMD, SERPINA1, ABCA4, CFTR, HEXA, RAB7A, ATP7B, HFE, LIPA, SCNN1A, PKD1, PKD2, PKHD1, ACE, ALB, VHL, EPO, PKD2, FH, ACE, TNF, SPP1, IL6, MYH9, PKD1, TSC2, ADIPOQ, IL2, CCL2, TGFB1, VHL, UMOD, BCOR, FLCN, FLCN, PKD1, TP53, CRP, PTEN, IFT88, CLDN14, FH, VHL, AGT, MET, MYH9, YWHAE, PKD1, HAMP, EPO, MUC1, BAP1, APOE, CYBA, GSTT1, IFNG, IGF1, IL2, ABCB1, SDHB, TP53, TSC2, BRAF, CDKN1B, GLA, KRT7, PPARG, RET, TRPC6, NDRG1, GANAB, NOX4, ADIPOR1, GREB1L, ANKS6, NUTM2B, CAT, CYBA, CYBB, EGFR, HMOX1, LRP2, SERPINE1, PAX2, ABCB1, PPARA, PPARG, PTGS2, RELA, RET, TLR4, UMOD, BAP1, RETN, GREB1L, FRAS1, CRB2, APRT, AXL, CCND1, BRAF, CBR1, CPT1A, CYP1A1, CYP2B6, EDN1, ERBB2, HMGCR, MME, NFKB1, NGF, MAPK1, MAPK3, PTGS2, PTGS2, HLTF, SOD1, SOD2, SREBF2, HNF1B, TERT, TNFSF10, NDRG1, MBTPS2, WNT4, BCOR, INF2, ALG9, BICC1, TMEM67, IRX2, FREM1, ANKS6, FREM2, CD46, COL4A3, COL4A4, COL4A5, TTC21B, NPHP4, CD2AP, CFI, LAMB2, LMX1B, or MYH9.

In some embodiments, the genes targets that may be encoded or targeted by a payload sequence and delivered to a tissue of a subject to treat, prevent, or alleviate symptoms of a disease or condition may be associated with a disease or disorder. For example, CNGA3 or CNGB3 associated with Achromatopsia; ABCD1 associated with Adrenomyeloneuropathy; UBE3A associated with Angelman Syndrome; Tafazzin associated with Barth Syndrome; CLN1, CLN2, CLN3, CLN4, CLN5, or CLN6 associated with Batten Disease; ASPA associated with Canavan Disease; PKD1 or PDK2 associated with Autosomal Dominant Polycistic Kidney Disease; CYP21A2 associated with Congenital Adrenal Hyperplasia; PMM2 associated with Congenital Disorder of Glycosylation 1a; LAMP2 associated with Danon Disease; GLA associated with Fabry Disease; GBA associated with Gaucher Disease; GLB1 associated with GM1 Gangliosidosis; G6PC associated with Glycogen SD 1a; F9 associated with Hemophilia; Serping1 associated with Hereditary Angioedema; GALC associated with Krabbe Disease; GUCY2D, NDI, ND6, ND4, RPE65, or AIPL1 associated with Leber's Disease; MTM1 associated with Myotubular Myopathy; I, II, IIA, IIC, IIID, IVA, VI, VII, and IXA associated with Mucopolysaccharidosis; OTC associated with Ornithine Transcarbamylase Deficiency; GAA associated with Pompe Disease; HEXB associated with Sandhoff Disease; SAM associated with Spinal Muscular Atrophy; HEXA associated with Tay-Sachs; USH2D-WHRN and USH3A-CLN1 associated with Usher Syndrome; SOD1 associated with ALS; HBB associated with Beta-thalassemia or Sickle Cell disease; BEST1 associated with Bestrophinopathy; CHM associated with Choroideremia; FXN associated with Freidreich's Ataxia; SLC37A4 associated with GSD1b; IGF1 associated with Osteoporosis; RPGR or RHO associated with Retinitis Pigmentosa; USH1C or CIB2 associated with Usher 1C, 1F; SERPINA1 associated with Alpha-I Antitrypsin Deficiency; MECP2 associated with Rett Syndrome; AC6, Serca2, VEGF-B, or PP1 associated with Heart Failure; GAD associated with Parkinson's Disease; MBTPS2 associated with Brain Anomalies, Retardation, Ectodermal Dysplasia, Skeletal Malformations, Hirschsprung Disease, Ear-Eye Anomalies, Cleft Palate-Cryptorchidism, And Kidney Dysplasia-Hypoplasia; CD46, COL4A3, COL4A4, COL4A5, TTC21B, NPHP4, CD2AP, CFI, LAMB2, LMX1B, or MYH9 associated with Chronic kidney disease/disorder with a monogenetic origin; TERT or IRX2 associated with Clear cell sarcoma of kidney; ERBB2 associated with Collecting Duct Carcinoma of the Kidney; FREM1 associated with Congenital absence of kidneys syndrome; TMEM67 associated with Cystic kidney; SOD1 associated with Kidney Calculi; EDN1 or MME associated with Kidney Failure; CPT1A, CYP2B6, HMGCR, NFKB1, NGF, or SREBF2 associated with Kidney Failure, Chronic; INF2 associated with Kidney Failure or Chronic kidney disease/disorder with a monogenetic origin; PTGS2, HLTF, NDRG1, or BCOR associated with Kidney Neoplasm; CYP1A1, MAPK1, MAPK3, PTGS2, SOD2, or TNFSF10 associated with Malignant neoplasm of kidney; ALG9 associated with Polycystic Kidney Disease, Potter Type I, with Microbrachycephaly, Hypertelorism, and Brachymelia; BICC1 or ANKS6 associated with Polycystic Kidney, Autosomal Dominant; WNT4 associated with Sex Reversal, Female, With Dysgenesis Of Kidneys, Adrenals, And Lungs; FREM2 associated with Unilateral agenesis of kidney; or HNF1B associated with Unilateral Multicystic Dysplastic Kidney.

In some embodiments, the payload encodes a therapeutic polynucleotide (e.g., a therapeutic RNA). In some embodiments the therapeutic payload encodes a therapeutic RNA, such as a guide RNA (including an engineered or synthetic guide RNA) for genome editing or for RNA editing. In some embodiments, the therapeutic payload encodes a tRNA or a modified tRNA (engineered or synthetic tRNA).

In some embodiments, the payload may encode a therapeutic polynucleotide (e.g., a therapeutic RNA or modified tRNA) that can target a gene target listed in TABLE 6. In some embodiments, a payload may comprise an open reading frame encoding a gene target listed in TABLE 6 and may encode a protein expressed by the associated gene. In some embodiments, a payload may encode a protein associated with a disease (e.g., Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease). In some embodiments, a payload may encode a therapeutic polynucleotide that targets a gene associated with a disease (e.g., Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease). In some embodiments, the targeted gene may encode ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof.

In some embodiments, a gene encoded by or targeted by a payload (e.g., a transgene or polynucleotide) may be transcribed in a target cell type or target cell state at a level that is at least about 1-fold, at least about 1.1-fold, at least about 1.2-fold, at least about 1.3-fold, at least about 1.4-fold, at least about 1.5-fold, at least about 2-fold, at least about 3-fold, at least about 4-fold, at least about 5-fold, at least about 10-fold, at least about 20-fold, at least about 30-fold, at least about 40-fold, at least about 50-fold, at least about 100-fold, at least about 150-fold, or at least about 200-fold a transcription level of the payload in a non-target cell type or non-target cell state.

### Delivery Vehicle

A polynucleotide (e.g., a recombinant polynucleotide) of the present disclosure (e.g., a polynucleotide comprising a promoter and a payload) may be delivered via a delivery vehicle. In some embodiments, the delivery vehicle is a vector, such as a viral vector. A vector may facilitate delivery of the polynucleotide into a cell to genetically modify the cell. In some examples, the vector comprises DNA, such as double stranded or single stranded DNA. In some examples, the delivery vector may be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the polynucleotide may be used to deliver the polynucleotide to a cell.

In some embodiments, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector may be a recombinant vector, a hybrid vector, a chimeric vector, a self-complementary vector, a single-stranded vector, or any combination thereof.

In some embodiments, the viral vector may be an adeno-associated virus (AAV). In some embodiments, the AAV may be any AAV known in the art. In some embodiments, the viral vector may be of a specific serotype. Adeno-associated virus (AAV) vectors include vectors derived from any AAV serotype, including, but not limited to AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68. In some embodiments, the viral vector may be a derivative of any of these serotypes, or a combination of serotypes.

In some embodiments, a polynucleotide is introduced into a subject by non-viral vector systems. In some embodiments, cationic lipids, polymers, hydrodynamic injection and/or ultrasound may be used in delivering a polynucleotide to a subject in the absence of virus.

In some examples, the vector may be a eukaryotic vector, a prokaryotic vector (e.g., a bacterial vector) a viral vector, or any combination thereof. In some examples, the vector may be a viral vector. In some embodiments, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector may be a recombinant vector, a hybrid vector, a chimeric vector, a self-complementary vector, a single-stranded vector, or any combination thereof.

In some embodiments, the viral vector may be an adeno-associated virus (AAV). In some embodiments, the AAV may be any AAV known in the art. In some embodiments, the viral vector may be of a specific serotype. In some embodiments, the viral vector may be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV-DJ serotype, AAV-DJ/8 serotype, AAV-DJ/9 serotype, AAV1/2 serotype, AAV.rh8 serotype, AAV.rh10 serotype, AAV.rh20 serotype, AAV.rh39 serotype, AAV.Rh43 serotype, AAV.Rh74 serotype, AAV.v66 serotype, AAV.Oligo001 serotype, AAV.SCH9 serotype, AAV.r3.45 serotype, AAV.RHM4-1 serotype, AAV.hu37 serotype, AAV.Anc80 serotype, AAV.Anc80L65 serotype, AAV.7m8 serotype, AAV.PhP.eB serotype, AAV.PhP.V1 serotype, AAV.PHP.B serotype, AAV.PhB.C1 serotype, AAV.PhB.C2 serotype, AAV.PhB.C3 serotype, AAV.PhB.C6 serotype, AAV.cy5 serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, AAV.HSC17 serotype, or AAVhu68 serotype, a derivative of any of these serotypes, or any combination thereof.

In some embodiments, the AAV vector may be a recombinant vector, a hybrid AAV vector, a chimeric AAV vector, a self-complementary AAV (scAAV) vector, a single-stranded AAV, or any combination thereof.

In some embodiments, the AAV vector may be a recombinant AAV (rAAV) vector. Methods of producing recombinant AAV vectors may be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and transgene sequences, etc. In some examples, the viral vectors described herein may be engineered through synthetic or other suitable means by references to published sequences, such as those that may be available in the literature. For example, the genomic and protein sequences of various serotypes of AAV, as well as the sequences of the native terminal repeats (TRs), Rep proteins, and capsid subunits may be known in the art and may be found in the literature or in public databases such as GenBank or Protein Data Bank (PDB).

In some examples, methods of producing delivery vectors herein comprising packaging a polynucleotide of the present disclosure (e.g., a polynucleotide comprising a promoter and a payload) in an AAV vector. In some examples, methods of producing the delivery vectors described herein comprise, (a) introducing into a cell: (i) a polynucleotide comprising a promoter and a payload disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the polynucleotide comprising a promoter and a payload disclosed herein in the AAV particle, thereby generating an AAV delivery vector. In some examples, any polynucleotide comprising a promoter and a payload disclosed herein may be packaged in the AAV vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector may be produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector may be a chimeric AAV vector. In some examples, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the AAV vector comprises a self-complementary AAV genome. Self-complementary AAV genomes may be generally known in the art and contain both DNA strands which can anneal together to form double-stranded DNA.

In some examples, the delivery vector may be a retroviral vector. In some examples, the retroviral vector may be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector may be transfected such that the majority of sequences coding for the structural genes of the virus (e.g., gag, pol, and env) may be deleted and replaced by the gene(s) of interest.

In some examples, the delivery vehicle may be a non-viral vector. Examples of non-viral vectors may include plasmids, lipid nanoparticles, lipoplexes, polymersomes, polyplexes, dendrimers, nanoparticles, and cell-penetrating peptides. The non-viral vector may comprise a polynucleotide, such as a plasmid, encoding for a promoter (e.g., comprising a cell type- or cell state-specific response element and a switchable core promoter) and a payload sequence. In some examples, the delivery vehicle may be a plasmid. In some examples, the plasmid may be a minicircle plasmid. In some embodiments, a vector may comprise naked DNA (e.g., a naked DNA plasmid). In some embodiments, the non-viral vector comprises DNA. In some embodiments, the non-viral vector comprises RNA. In some examples, the non-viral vector comprises circular double-stranded DNA. In some examples, the non-viral vector may comprise a linear polynucleotide. In some examples, the non-viral vector comprises a polynucleotide encoding one or more genes of interest and one or more regulatory elements. In some examples, the non-viral vector comprises a bacterial backbone containing an origin of replication and an antibiotic resistance gene or other selectable marker for plasmid amplification in bacteria. In some examples, the non-viral vector contains one or more genes that provide a selective marker to induce a target cell to retain a polynucleotide (e.g., a plasmid) of the non-viral vector. In some examples, the non-viral vector may be formulated for delivery through injection by a needle carrying syringe. In some examples, the non-viral vector may be formulated for delivery via electroporation. In some examples, a polynucleotide of the non-viral vector may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

In some embodiments, the vector containing the polynucleotide is a non-viral vector system. In some embodiments, the non-viral vector system comprises cationic lipids, or polymers. In some embodiments, the polynucleotide or a non-viral vector comprising the polynucleotide is delivered to a cell by hydrodynamic injection or ultrasound.

In some embodiments, a viral vector may be an engineered for fine-tuned transgene expression utilizing transcriptional control (e.g., using an engineered promoter for cell state specific expression) and translational control (e.g., 5′UTR, 3′UTR, and coding region of the polynucleotide encoding the transgene), as illustrated in FIG. 12.

### Methods of Expressing a Payload in a Target Cell

A polynucleotide (e.g., a recombinant polynucleotide) of the present disclosure may be used in a method of expressing a payload (e.g., a transgene or polynucleotide) in a target cell. A method of expressing a payload in a cell may comprise delivering a polynucleotide encoding the payload to one or more cells, including one or more target cells, and expressing the payload in the target cell. The target cell may be a target cell type (e.g., a neuron, a hepatocyte, a retinal cell, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast). The target cell may comprise a genetic variation of interest. The target cell may express a protein from a genetic variation of interest (e.g., a mutant protein from a gene comprising a mutation associated with a disease). The target cell may comprise a phenotype of interest (e.g., a disease phenotype).

In some embodiments, the payload is expressed in a cell state-dependent manner. For example, a payload may be transcribed in the target cell type at higher levels than in non-target cell types. In another example, a payload may be transcribed in a cell comprising a genetic variation of interest at higher levels than in cells lacking the genetic variation. In another example, a payload may be transcribed in a cell having a phenotype of interest at higher levels than in cells lacking the phenotype. As described herein, a promoter sequence of the polynucleotide may be engineered for cell state-specific transcription of the encoded payload. For example, the promoter sequence may be engineered to promote increased transcription of the payload in a target cell relative to a non-target cell. In some embodiments, a method of expressing a payload may comprise delivering a polynucleotide to a cell using a vector (e.g., a viral vector), as described herein.

### Methods of Treatment

A polynucleotide (e.g., a recombinant polynucleotide) of the present disclosure may be used in a method of treating a disorder in a subject in need thereof. A disorder may be a disease, a condition, a genotype, a phenotype, or any state associated with an adverse effect. In some embodiments, treating a disorder may comprise preventing, slowing progression of, reversing, or alleviating symptoms of the disorder. A method of treating a disorder may comprise delivering a polynucleotide encoding a payload to a cell of a subject in need thereof and expressing the payload in the cell. In some embodiments, the payload is expressed in a cell state-dependent manner. For example, a payload may be transcribed in a target cell type at higher levels than in non-target cell types. In another example, a payload may be transcribed in a cell comprising a genetic variation of interest at higher levels than in cells lacking the genetic variation. In another example, a payload may be transcribed in a cell expressing a protein from a gene comprising a genetic variation of interest at higher levels than in cells lacking expression of the protein from a gene comprising a genetic variation of interest. In another example, a payload may be transcribed in a cell having a phenotype of interest at higher levels than in cells lacking the phenotype. As described herein, a promoter sequence of the polynucleotide may be engineered for cell state-specific transcription of the encoded payload. In some embodiments, a method of treatment may comprise delivering a polynucleotide to a subject using a vector (e.g., a viral vector), as described herein.

In some embodiments, a polynucleotide (e.g., a recombinant polynucleotide) of the present disclosure may be used to treat a genetic disorder. For example, a genetic disorder caused by a mutation in or altered expression of a protein may be treated by delivering a polynucleotide encoding a wild type copy of the protein to a cell of the subject and expressing the protein in a target cell state (e.g., a target cell type, a cell having the genetic mutation, a cell expressing a protein from a gene having the genetic mutation, and/or a cell having a phenotype associated with the genetic mutation). The wild type protein encoded by the payload may be expressed in the target cells, thereby treating the genetic disorder. In another example, a genetic disorder caused by a mutation of a gene may be treated by delivering a polynucleotide encoding a gRNA targeting the mutated gene sequence to a cell of a subject comprising the mutated sequence in a target cell state or expressing a protein from the mutated sequence in the target cell state (e.g., a target cell type, a cell having the genetic mutation, a cell expressing a protein from a gene having the genetic mutation, and/or a cell having a phenotype associated with the genetic mutation). The gRNA may be expressed in the target cell and may target the mutated gene for gene editing, thereby treating the genetic disorder. In some embodiments, a polynucleotide of the present disclosure may be used to treat a condition associated with one or more mutations in a subset of cells. For example, a cancer caused by mutations in a subset of cells may be treated by delivering a polynucleotide encoding a pro-apoptotic factor to a cell of a subject and selectively transcribing the pro-apoptotic factor in the cancer cells.

In some embodiments, a polynucleotide (e.g., a recombinant polynucleotide) of the present disclosure may be used in a method to treat a genetic disorder, a neuronal disorder, cancer, or an eye disorder. Examples of disorders that may be treated using a polynucleotide of the present disclosure are provided in TABLE 6. For example, Rett syndrome may be treated by delivering a polynucleotide encoding a wild type MeCP2 to a subject in need thereof and selectively transcribing the MECP2 gene in neurons expressing a mutant MeCP2 protein and exhibiting the disease phenotype associated with the mutation in MECP2. For example, Rett syndrome may be treated by delivering a polynucleotide encoding a wild type MeCP2 to a subject in need thereof and selectively transcribing the MECP2 gene in neurons expressing a protein from a mutant MECP2 and exhibiting the disease phenotype associated with the mutant MECP2. The transcription level of the MECP2 gene may be tuned to prevent over-expression that may cause seizures. Delivery of the polynucleotide may reduce the symptoms of Rett syndrome in the subject. In another example, frontotemporal dementia may be treated by delivering a polynucleotide encoding a wild type progranulin gene to a subject in need thereof and selectively transcribing the progranulin gene in neurons. Delivery of the polynucleotide may slow progression of or reduce symptoms of frontotemporal dementia.

In some embodiments, disorders that may be treated by delivering a polynucleotide of the present disclosure to a subject in need thereof include Rett syndrome, MECP2 duplication syndrome, Frontotemporal dementia, neuronal ceroid lipofuscinosis, Retinitis Pigmentosa 7, macular degeneration, Retinitis Pigmentosa 4, Angelman Syndrome, DYRK1A haploinsufficiency, MEF2C haploinsufficiency syndrome, Sotos syndrome, Reverse Sotos syndrome, Alpha-thalassemia X-linked intellectual disability syndrome, Xp22.12 duplication, Coffin-Lowry syndrome, Pitt Hopkins syndrome, Mowat-Wilson Syndrome, FOXG1 syndrome, CDKL5 deficiency disorder, West Syndrome, 2q23.1 microdeletion syndrome, Doose Syndrome, SLC6A1 epileptic encephalopathy, Duchenne's muscular dystrophy, Becker muscular dystrophy, Alpha-1 antitrypsin deficiency (AATD), Macular Degeneration/Stargardt disease, Cystic Fibrosis, Tay-Sachs, Charcot-Marie-Tooth neuropathy, Wilson's disease, Hereditary Hemochromatosis, Wolman disease, cholesteryl ester storage disease, Psueodhypoaldosteronism type 1, Achromatopsia, Adrenomyeloneuropathy, Barth Syndrome, Batten Disease, Canavan Disease; PKDJ or PDK2 associated with Autosomal Dominant Polycistic Kidney Disease, Congenital Adrenal Hyperplasia, Congenital Disorder of Glycosylation 1a, Danon Disease, Fabry Disease, Gaucher Disease, GM1 Gangliosidosis, Glycogen SD 1a, Hemophilia, Hereditary Angioedema, Krabbe Disease, Leber's Disease, Myotubular Myopathy, Mucopolysaccharidosis, Ornithine Transcarbamylase Deficiency, Pompe Disease, Sandhoff Disease, Spinal Muscular Atrophy, Tay-Sachs, Usher Syndrome, ALS, Beta-thalassemia or Sickle Cell disease, Bestrophinopathy, Choroideremia, Freidreich's Ataxia, GSD1b, Osteoporosis, Alpha-1 Antitrypsin Deficiency, Heart Failure, Parkinson's Disease, Brain Anomalies, Retardation, Ectodermal Dysplasia, Skeletal Malformations, Hirschsprung Disease, Ear-Eye Anomalies, Cleft Palate-Cryptorchidissm, And Kidney Dysplasia-Hypoplasia, Chronic kidney disease/disorder with a monogenetic origin, Clear cell sarcoma of kidney, Collecting Duct Carcinoma of the Kidney, Congenital absence of kidneys syndrome, Cystic kidney, Kidney Calculi, Kidney Failure, Kidney Neoplasm, Malignant neoplasm of kidney, Polycystic Kidney Disease, Potter Type I, with Microbrachycephaly, Hypertelorism, and Brachymelia, Polycystic Kidney, Autosomal Dominant, Sex Reversal, Female, With Dysgenesis Of Kidneys, Adrenals, And Lungs, Unilateral agenesis of kidney, or Unilateral Multicystic Dysplastic Kidney.

### Pharmaceutical Compositions

The compositions described herein (e.g., compositions comprising a polynucleotide, such as a recombinant polynucleotide) may be formulated with a pharmaceutically acceptable carrier for administration to a subject (e.g., a human or a non-human animal). A pharmaceutically acceptable carrier may include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers, and adjuvants consistent with the compositions of the present disclosure may be found in, for example, Remington's Pharmaceutical Sciences, 21st Ed., Mack Publ. Co., Easton, Pa. (2005), incorporated herein by reference in its entirety.

Pharmaceutical compositions for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In some embodiments, the polynucleotide (e.g., a recombinant polynucleotide) of the present disclosure or recombinant polynucleotide cassette of the present disclosure may be administered to cells via a lipid nanoparticle. In some embodiments, the lipid nanoparticle may be administered at the appropriate concentration according to standard methods appropriate for the target cells.

In some embodiments, the polynucleotide (e.g., a recombinant polynucleotide) of the present disclosure or recombinant polynucleotide cassette of the present disclosure may be administered to cells via a viral vector. In some embodiments, the viral vector may be administered at the appropriate multiplicity of infection according to standard transduction methods appropriate for the target cells. Titers of the virus vector or capsid to administer can vary depending on the target cell type or cell state and number and can be determined by those of skill in the art. In some embodiments, at least about 10² infections units are administered. In some embodiments, at least about 10³, 10⁴, 10⁵, 10⁶, 10⁷, 10⁸, 10⁹, 10¹⁰, 10¹¹, 10¹², or 10¹³ infectious units are administered.

In some embodiments, the polynucleotide (e.g., a recombinant polynucleotide) or recombinant polynucleotide cassette is introduced to cells of any type or state, including, but not limited to neural cells, cells of the eye (including retinal cells, retinal pigment epithelium, and corneal cells), lung cells, epithelial cells, skeletal muscle cells, dendritic cells, hepatic cells, pancreatic cells, bone cells, hematopoietic stem cells, spleen cells, keratinocytes, fibroblasts, endothelial cells, prostate cells, and heart cells.

In some embodiments, the polynucleotide (e.g., a recombinant polynucleotide) of the disclosure or the recombinant polynucleotide cassette of the disclosure may be introduced to cells in vitro via a viral vector for administration of modified cells to a subject. In some embodiments, a viral vector encoding the polynucleotide of the disclosure or the recombinant polynucleotide cassette of the disclosure is introduced to cells that have been removed from a subject. In some embodiments, the modified cells are placed back in the subject following introduction of the viral vector.

In some embodiments, a dose of modified cells is administered to a subject according to the age and species of the subject, disease or disorder to be treated, as well as the cell type or state and mode of administration. In some embodiments, at least about 10²-10⁸ cells are administered per dose. In some embodiments, cells transduced with viral vector are administered to a subject in an effective amount.

In some embodiments, the dose of viral vector administered to a subject will vary according to the age of the subject, the disease or disorder to be treated, and mode of administration. In some embodiments, the dose for achieving a therapeutic effect is a virus titer of at least about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or more transducing units.

Administration of the pharmaceutically useful polynucleotide of the present disclosure or the polynucleotide cassette of the present disclosure is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., Mack Publ. Co., Easton, Pa. (2005), incorporated herein by reference in its entirety.

As used herein, the term “therapeutic polynucleotide” may refer to a polynucleotide that is introduced into a cell and is capable of being expressed in the cell or to a polynucleotide that may, in itself, have a therapeutic activity, such as a gRNA or a tRNA.

As used herein, the term “polynucleotide” may refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

### NUMBERED EMBODIMENTS

### First Set of Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A recombinant polynucleotide comprising a promoter and a payload, wherein the promoter comprises: a transcription factor binding polynucleotide capable of binding to a transcription factor, and a core promoter capable of binding to or recruiting a polymerase; wherein the payload comprises a coding sequence encoding a protein. 2. The recombinant polynucleotide of embodiment 1, wherein the transcription factor is selected from ESRRG, RORB, NFIC, NFIA, NEUROD2, TBR1, or ZNF436. 3. The recombinant polynucleotide of embodiment 1 or embodiment 2, wherein the transcription factor is any one of the transcription factors provided in TABLE 1. 4. The recombinant polynucleotide of any one of embodiments 1-3, wherein the transcription factor binding polynucleotide comprises a first transcription factor binding motif capable of binding the transcription factor. 5. The recombinant polynucleotide of embodiment 4, wherein the first transcription factor binding motif is a consensus transcription factor binding motif 6. The recombinant polynucleotide of embodiment 4, wherein the first transcription factor binding motif is a variant transcription factor binding motif. 7. The recombinant polynucleotide of any one of embodiments 4-6, further comprising a second transcription factor binding motif capable of binding a second transcription factor. S. The recombinant polynucleotide of embodiment 7, wherein the second transcription factor binding motif is the same as the first transcription factor binding motif. 9. The recombinant polynucleotide of embodiment 7, wherein the second transcription factor binding motif is different than the first transcription factor binding motif. 10. The recombinant polynucleotide of any one of embodiments 4-9, wherein the transcription factor binding polynucleotide further comprises a third transcription factor binding motif capable of binding a third transcription factor. 11. The recombinant polynucleotide of any one of embodiments 1-10, wherein the transcription factor binding polynucleotide comprises 1, 2, 3, 4, 5, or 6 transcription factor binding motifs. 12. The recombinant polynucleotide of any one of embodiments 1-11, wherein the transcription factor binding polynucleotide is capable of binding 1, 2, 3, 4, 5, or 6 transcription factors. 13. The recombinant polynucleotide of any one of embodiments 1-12, wherein the core promoter comprises a TATA box, an RNA polymerase binding sequence, a B recognition element, a CCAAT box, a Pribnow box, or a sequence provided in TABLE 2. 14. The recombinant polynucleotide of any one of embodiments 1-13, wherein the polymerase is an RNA polymerase II. 15. The recombinant polynucleotide of any one of embodiments 1-14, wherein the coding sequence is capable of being transcribed by the polymerase upon binding of the transcription factor to the transcription factor binding polynucleotide and binding or recruitment of the polymerase to the core promoter. 16. The recombinant polynucleotide of any one of embodiments 1-15, wherein the protein is a neuronal protein, a retinal protein, a muscle protein, or an apoptosis-inducing protein. 17. The recombinant polynucleotide of any one of embodiments 1-16, wherein the protein is associated with a genetic disorder, a neuronal disorder, an eye disorder, a muscular disorder, or a cancer. 18. The recombinant polynucleotide of any one of embodiments 1-17, wherein the protein is MeCP2, progranulin, dystrophin, or peripherin 2. 19. The recombinant polynucleotide of any one of embodiments 1-18, wherein the protein is encoded by any one of the genes provided in TABLE 3. 20. The recombinant polynucleotide of any one of embodiments 1-19, wherein the promoter is engineered to control a transcription level of the payload. 21. The recombinant polynucleotide of embodiment 20, wherein the transcription level is cell state specific. 22. The recombinant polynucleotide of embodiment 21, wherein the cell state is a cell type, a cell genotype, a cell phenotype, or any combination thereof. 23. An engineered viral vector comprising the recombinant polynucleotide of any one of embodiments 1-22 in a viral vector. 24. The engineered viral vector of embodiment 23, wherein the viral vector is an adenoviral vector, an adeno-associated viral vector, or a lentivector. 25. The engineered viral vector of embodiment 24, wherein the adeno-associated viral vector is selected from the group consisting of AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. 26. The engineered viral vector of any one of embodiments 23-25, wherein a viral capsid of the viral vector is from a first viral vector and a viral inverted terminal repeat sequence of the viral vector is from a second viral vector. 27. The engineered viral vector of embodiment 26, wherein the first viral vector, the second viral vector, or both is an adeno-associated viral vector. 28. The engineered viral vector of embodiment 26 or embodiment 27, wherein the first viral vector, the second viral vector, or both is selected from the group consisting of AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68. 29. A pharmaceutical composition comprising the recombinant polynucleotide of any one of embodiments 1-22 or the viral vector of any one of embodiments 23-28 and a pharmaceutically acceptable carrier. 30. A method of treating a disorder in a subject in need thereof, the method comprising: administering to the subject a composition comprising the recombinant polynucleotide of any one of embodiments 1-22, the viral vector of any one of embodiments 23-28, or the pharmaceutical composition of embodiment 29; and expressing the protein encoded by the recombinant polynucleotide in a target cell of the subject, thereby treating the disorder. 31. The method of embodiment 30, wherein the coding sequence is transcribed upon binding of the transcription factor to the transcription factor binding site and recruitment of the polymerase to the core promoter. 32. The method of embodiment 30 or embodiment 31, wherein a level of transcription of the coding sequence is higher in the target cell than in a non-target cell of the subject. 33. The method of embodiment 32, wherein the transcription factor is present at a higher level in the target cell than in the non-target cell. 34. The method of embodiment 32 or embodiment 33, wherein the non-target cell is a healthy cell. 35. The method of any one of embodiments 30-34, wherein the target cell is a neuron, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 36. The method of any one of embodiments 30-35, wherein the target cell is a diseased cell. 37. The method of embodiment 36, wherein the diseased cell comprises a genetic mutation associated with the disorder and has a disease phenotype associated with the genetic mutation. 38. The method of embodiment 36 or embodiment 37, wherein the diseased cell comprises a mutation in MECP2, GRN, PRPH2, or DMX. 39. The method of any one of embodiments 36-38, wherein the diseased cell comprises a mutation in any one of the genes provided in TABLE 3. 40. The method of embodiment 36 or embodiment 37, wherein the diseased cell is a cancer cell. 41. The method of any one of embodiments 30-40, wherein the disorder is a genetic disorder, a neuronal disorder, an eye disorder, a muscular disorder, or a cancer. 42. The method of embodiment 41, wherein the neuronal disorder is Rett syndrome or frontotemporal dementia. 43. The method of any one of embodiments 30-42, wherein the disorder is any one of the disorders provided in TABLE 3. 44. A method of expressing a protein in a target cell, the method comprising: administering to the subject a composition comprising the recombinant polynucleotide of any one of embodiments 1-22, the viral vector of any one of embodiments 23-28, or the pharmaceutical composition of embodiment 29; transcribing the coding sequence in the target cell; and expressing the protein encoded by the coding sequence in the target cell. 45. The method of embodiment 44, wherein the coding sequence is transcribed upon binding of the transcription factor to the transcription factor binding site and recruitment of the polymerase to the core promoter. 46. The method of embodiment 44 or embodiment 45, wherein a level of transcription of the coding sequence is higher in the target cell than in a non-target cell. 47. The method of embodiment 46, wherein a level of expression of the protein is higher in the target cell than in the non-target cell. 48. The method of embodiment 46 or embodiment 47, wherein the transcription factor is present at a higher level in the target cell than in the non-target cell. 49. The method of any one of embodiments 44-48, wherein the target cell is a neuron, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast.

### Second Set of Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A recombinant transcription factor binding polynucleotide comprising a sequence having at least 80%, at least 85%, at least 90%, or at least 95% sequence identity to any one of SEQ ID NO: 26-SEQ ID NO: 41. 2. The recombinant transcription factor binding polynucleotide of embodiment 1 comprising the sequence of any one of SEQ ID NO: 26-SEQ ID NO: 41. 3. The recombinant transcription factor binding polynucleotide of embodiment 1 or embodiment 2, wherein the recombinant transcription factor binding polynucleotide is capable of binding to a transcription factor. 4. The recombinant transcription factor binding polynucleotide of embodiment 3, wherein the transcription factor is expressed more highly in a target cell than in a non-target cell. 5. The recombinant transcription factor binding polynucleotide of embodiment 4, wherein the target cell is a mutant cell having a disease phenotype associated with a genetic mutation in a gene, and wherein the non-target cell is a wild type cell having a wild type phenotype associated with a wild type allele in the gene. 6. The recombinant transcription factor binding polynucleotide of embodiment 5, wherein the mutant cell expresses a mutant MeCP2 protein, and wherein the wild type cell expresses a wild type MeCP2 protein. 7. The recombinant transcription factor binding polynucleotide of embodiment 5 or embodiment 6, wherein the target cell expresses a mutant MeCP2 protein, and wherein the non-target cell expresses a wild type MeCP2 protein. 8. A recombinant polynucleotide comprising a promoter and a payload, wherein the promoter comprises: a transcription factor binding polynucleotide capable of binding to a transcription factor, wherein the transcription factor binding polynucleotide comprises the recombinant transcription factor binding polynucleotide of any one of embodiments 1-6, and a core promoter capable of recruiting a polymerase; wherein the payload comprises a coding sequence. 9. The recombinant polynucleotide of embodiment 8, wherein the promoter comprises a sequence having at least 80%, at least 85%, at least 90%, or at least 95% sequence identity to any one of SEQ ID NO: 113-SEQ ID NO: 140. 10. The recombinant polynucleotide of embodiment 8 or embodiment 9, wherein the promoter comprises a sequence having at least 80%, at least 85%, at least 90%, or at least 95% sequence identity to SEQ ID NO: 115. 11. The recombinant polynucleotide of any one of embodiments 8-10, wherein the promoter comprises a sequence of any one of SEQ ID NO: 113-SEQ ID NO: 140. 12. The recombinant polynucleotide of any one of embodiments 8-11, wherein the promoter comprises a sequence of SEQ ID NO: 115. 13. A recombinant polynucleotide comprising a promoter and a payload, wherein the promoter comprises: a transcription factor binding polynucleotide capable of binding to a transcription factor, wherein the transcription factor binding polynucleotide comprises at least three transcription factor binding motifs, and a core promoter capable of recruiting a polymerase; wherein the payload comprises a coding sequence. 14. The recombinant polynucleotide of embodiment 13, wherein the transcription factor is selected from ESRRG, RORB, NFIC, NFIA, NEUROD2, TBR1, or ZNF436. 15. The recombinant polynucleotide of embodiment 13 or embodiment 14, wherein the transcription factor is a transcription factor provided in TABLE 1. 16. The recombinant polynucleotide of any one of embodiments 13-15, wherein the at least three transcription factor binding motifs comprise a first transcription factor binding motif capable of binding the transcription factor. 17. The recombinant polynucleotide of embodiment 16, wherein the first transcription factor binding motif is a consensus transcription factor binding motif. 18. The recombinant polynucleotide of embodiment 16, wherein the first transcription factor binding motif is a variant transcription factor binding motif. 19. The recombinant polynucleotide of any one of embodiments 16-18, wherein the first transcription factor binding motif has at least 80%, at least 85%, at least 90%, or at least 95% sequence identity to a transcription factor binding motif provided in TABLE 2. 20. The recombinant polynucleotide of any one of embodiments 16-19, wherein the first transcription factor binding motif is selected from a transcription factor binding motif provided in TABLE 2. 21. The recombinant polynucleotide of any one of embodiments 16-20, wherein the at least three transcription factor binding motifs comprise a second transcription factor binding motif capable of binding a second transcription factor. 22. The recombinant polynucleotide of embodiment 21, wherein the second transcription factor binding motif is the same as the first transcription factor binding motif. 23. The recombinant polynucleotide of embodiment 21, wherein the second transcription factor binding motif is different than the first transcription factor binding motif. 24. The recombinant polynucleotide of any one of embodiments 21-23, wherein the second transcription factor binding motif has at least 80%, at least 85%, at least 90%, or at least 95% sequence identity to a transcription factor binding motif provided in TABLE 2. 25. The recombinant polynucleotide of any one of embodiments 21-24, wherein the second transcription factor binding motif is selected from a transcription factor binding motif provided in TABLE 2. 26. The recombinant polynucleotide of any one of embodiments 16-25, wherein the at least three transcription factor binding motifs comprise a third transcription factor binding motif capable of binding a third transcription factor. 27. The recombinant polynucleotide of embodiment 26, wherein the third transcription factor binding motif is the same as the first transcription factor binding motif, the second transcription factor binding motif, or both. 28. The recombinant polynucleotide of embodiment 26, wherein the third transcription factor binding motif is different than the first transcription factor binding motif, the second transcription factor binding motif, or both. 29. The recombinant polynucleotide of any one of embodiments 26-28, wherein the third transcription factor binding motif has at least 80%, at least 85%, at least 90%, or at least 95% sequence identity to a transcription factor binding motif provided in TABLE 2. 30. The recombinant polynucleotide of any one of embodiments 26-29, wherein the third transcription factor binding motif is selected from a transcription factor binding motif provided in TABLE 2. 31. The recombinant polynucleotide of any one of embodiments 13-30, wherein the transcription factor binding polynucleotide comprises 3, 4, 5, or 6 transcription factor binding motifs. 32. The recombinant polynucleotide of any one of embodiments 13-31, wherein the transcription factor binding polynucleotide is capable of binding 1, 2, 3, 4, 5, or 6 transcription factors. 33. The recombinant polynucleotide of any one of embodiments 13-32, wherein the transcription factor binding polynucleotide comprises at least four transcription factor binding motifs. 34. The recombinant polynucleotide of embodiment 33, wherein the at least four transcription factor binding motifs comprise a first transcription factor binding motif, a second transcription factor binding motif, a third transcription factor binding motif, and a fourth transcription factor binding motif. 35. The recombinant polynucleotide of embodiment 34, wherein the first transcription factor binding motif is the same as the third transcription factor binding motif 36. The recombinant polynucleotide of embodiment 34 or embodiment 35, wherein the second transcription factor binding motif is the same as the fourth transcription factor binding motif. 37. The recombinant polynucleotide of any one of embodiments 34-36, wherein the first transcription factor binding motif, the second transcription factor binding motif, the third transcription factor binding motif, and the fourth transcription factor binding motif are the same. 38. The recombinant polynucleotide of any one of embodiments 13-37, wherein the transcription factor binding polynucleotide has at least 80%, at least 85%, at least 90%, or at least 95% sequence identity to a transcription factor binding sequence provided in TABLE 3. 39. The recombinant polynucleotide of any one of embodiments 13-38, wherein the transcription factor binding polynucleotide is selected from a transcription factor binding sequence provided in TABLE 3. 40. The recombinant polynucleotide of any one of embodiments 8-39, wherein the core promoter comprises a TATA box, an initiator sequence, an RNA polymerase binding sequence, a B recognition element, a CCAAT box, a Pribnow box, a sequence provided in TABLE 4, or combinations thereof. 41. The recombinant polynucleotide of any one of embodiments 8-40, wherein the polymerase is an RNA polymerase II. 42. The recombinant polynucleotide of any one of embodiments 8-41, wherein the promoter has at least 80%, at least 85%, at least 90%, or at least 95% sequence identity to a promoter sequence provided in TABLE 5. 43. The recombinant polynucleotide of any one of embodiments 8-42, wherein the promoter is selected from a promoter sequence provided in TABLE 5. 44. The recombinant polynucleotide of any one of embodiments 8-43, wherein the coding sequence is capable of being transcribed by the polymerase upon binding of the transcription factor to the transcription factor binding polynucleotide and recruitment of the polymerase to the core promoter. 45. The recombinant polynucleotide of any one of embodiments 8-44, wherein the coding sequence encodes a protein. 46. The recombinant polynucleotide of embodiment 45, wherein the protein is a neuronal protein, a retinal protein, a muscle protein, or an apoptosis-inducing protein. 47. The recombinant polynucleotide of embodiment 45 or embodiment 46, wherein the protein is associated with a genetic disorder, a neuronal disorder, an eye disorder, a muscular disorder, or a cancer. 48. The recombinant polynucleotide of any one of embodiments 45-47, wherein the protein is MeCP2, progranulin, dystrophin, or peripherin 2. 49. The recombinant polynucleotide of any one of embodiments 45-48, wherein the protein is encoded by any one of the genes provided in TABLE 6. 50. The recombinant polynucleotide of any one of embodiments 8-44, wherein the coding sequence encodes a therapeutic polynucleotide. 51. The recombinant polynucleotide of embodiment 50, wherein the therapeutic polynucleotide is a gRNA or a tRNA. 52. The recombinant polynucleotide of embodiment 50 or embodiment 51, wherein the therapeutic polynucleotide targets a gene associated with a genetic disorder, a neuronal disorder, an eye disorder, a muscular disorder, or a cancer. 53. The recombinant polynucleotide of any one of embodiments 50-52, wherein the therapeutic polynucleotide targets a gene provided in TABLE 6. 54. The recombinant polynucleotide of any one of embodiments 8-53, wherein the promoter is engineered to control a transcription level of the payload. 55. The recombinant polynucleotide of embodiment 54, wherein the transcription level is cell state-specific. 56. The recombinant polynucleotide of embodiment 54 or embodiment 55, wherein the transcription level is cell type-specific. 57. The recombinant polynucleotide of any one of embodiments 54-56, wherein the transcription level is cell genotype-specific. 58. The recombinant polynucleotide of any one of embodiments 54-57, wherein a transcriptional level in a target cell is at least 1-fold, at least 1.1-fold, at least 1.2-fold, at least 1.3-fold, at least 1.4-fold, at least 1.5-fold, at least 2-fold, at least 2.5-fold, at least 3-fold, or at least 4-fold a transcriptional level in a non-target cell. 59. An engineered viral vector comprising the recombinant polynucleotide of any one of embodiments 8-58 in a viral vector. 60. The engineered viral vector of embodiment 59, wherein the viral vector is an adenoviral vector, an adeno-associated viral vector, or a lentivector. 61. The engineered viral vector of embodiment 60, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68. 62. The engineered viral vector of any one of embodiments 59-61, wherein a viral capsid of the viral vector is from a first viral vector and a viral inverted terminal repeat sequence of the viral vector is from a second viral vector. 63. The engineered viral vector of embodiment 62, wherein the first viral vector, the second viral vector, or both is an adeno-associated viral vector. 64. The engineered viral vector of embodiment 62 or embodiment 63, wherein the first viral vector, the second viral vector, or both is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68. 65. A pharmaceutical composition comprising the recombinant polynucleotide of any one of embodiments 16-57, or the viral vector of any one of embodiments 59-64 and a pharmaceutically acceptable carrier. 66. A method of treating a disorder in a subject in need thereof, the method comprising administering to the subject a composition comprising the recombinant polynucleotide of any one of embodiments 16-58, the viral vector of any one of embodiments 59-64, or the pharmaceutical composition of embodiment 65, thereby treating the disorder. 67. The method of embodiment 66, wherein the coding sequence is transcribed upon binding of the transcription factor to the transcription factor binding site and recruitment of the polymerase to the core promoter. 68. The method of embodiment 66 or embodiment 67, wherein a level of transcription of the coding sequence is higher in the target cell than in a non-target cell of the subject. 69. The method of embodiment 68, wherein the target cell is a diseased cell having a disease phenotype associated with expression of a mutant MeCP2 protein, and the non-target cell is a healthy cell having a wild type phenotype associated with expression of a wild type MeCP2 protein. 70. The method of embodiment 68 or embodiment 69, wherein the transcription factor is present at a higher level in the target cell than in the non-target cell. 71. The method of embodiment 70, wherein the transcription factor is more active in the target cell than in the non-target cell. 72. The method of any one of embodiments 68-71, wherein the non-target cell is a healthy cell. 73. The method of any one of embodiments 66-72, wherein the target cell is a neuron, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 74. The method of any one of embodiments 66-73, wherein the target cell is a diseased cell. 75. The method of embodiment 74, wherein the diseased cell comprises a genetic mutation associated with the disorder and has a disease phenotype associated with the genetic mutation. 76. The method of embodiment 74 or embodiment 75, wherein the diseased cell comprises a mutation in MECP2, GRN, PRPH2, or DMX. 77. The method of any one of embodiments 74-76, wherein the diseased cell comprises a mutation in any one of the genes provided in TABLE 6. 78. The method of embodiment 74 or embodiment 75, wherein the diseased cell is a cancer cell. 79. The method of any one of embodiments 66-78, wherein the disorder is a genetic disorder, a neuronal disorder, an eye disorder, a muscular disorder, or a cancer. 80. The method of embodiment 79, wherein the neuronal disorder is Rett syndrome or frontotemporal dementia. 81. The method of any one of embodiments 66-80, wherein the disorder is any one of the disorders provided in TABLE 6. 82. The method of any one of embodiments 66-81, further comprising expressing a protein encoded by the coding sequence in the target cell. 83. The method of embodiment 82, wherein the protein is a neuronal protein, a retinal protein, a muscle protein, or an apoptosis-inducing protein. 84. The method of embodiment 82 or embodiment 83, wherein the protein is associated with a genetic disorder, a neuronal disorder, an eye disorder, a muscular disorder, or a cancer. 85. The method of any one of embodiments 82-84, wherein the protein is MeCP2, progranulin, dystrophin, or peripherin 2. 86. The method of any one of embodiments 82-85, wherein the protein is encoded by any one of the genes provided in TABLE 6. 87. The method of any one of embodiments 66-81, further comprising expressing a therapeutic polynucleotide encoded by the coding sequence in the target cell. 88. The method of embodiment 87, wherein the therapeutic polynucleotide is a gRNA or a tRNA. 89. The method of embodiment 87 or embodiment 88, wherein the therapeutic polynucleotide targets a gene associated with a genetic disorder, a neuronal disorder, an eye disorder, a muscular disorder, or a cancer. 90. The method of any one of embodiments 87-89, wherein the therapeutic polynucleotide targets a gene provided in TABLE 6. 91. A method of expressing a coding sequence in a target cell, the method comprising administering to the subject a composition comprising the recombinant polynucleotide of any one of embodiments 16-58, the viral vector of any one of embodiments 59-64, or the pharmaceutical composition of embodiment 65, thereby expressing the coding sequence in the target cell. 92. The method of embodiment 91, wherein the coding sequence is transcribed upon binding of the transcription factor to the transcription factor binding site and recruitment of the polymerase to the core promoter. 93. The method of embodiment 92, wherein the transcription factor is present at a higher level in the target cell than in the non-target cell. 94. The method of embodiment 93, wherein the target cell is a diseased cell having a disease phenotype associated with expression of a mutant MeCP2 protein, and the non-target cell is a healthy cell having a wild type phenotype associated with expression of a wild type MeCP2 protein. 95. The method of any one of embodiments 91-94, wherein the target cell is a neuron, a retinal cell, a hepatocyte, an epithelial cell, a muscle cell, an erythrocyte, a platelet, a bone marrow cell, an endothelial cell, an epidermal cell, a lymphocyte, a glial cell, an interstitial cell, an adipocyte, or a fibroblast. 96. The method of any one of embodiments 91-95, wherein a level of transcription of the coding sequence is higher in the target cell than in a non-target cell. 97. The method of any one of embodiments 91-96, further comprising expressing a protein encoded by the coding sequence in the target cell. 98. The method of embodiment 97, wherein a level of expression of the protein is higher in the target cell than in the non-target cell. 99. The method of embodiment 97 or embodiment 98, wherein the protein is a neuronal protein, a retinal protein, a muscle protein, or an apoptosis-inducing protein. 100. The method of any one of embodiments 97-99, wherein the protein is associated with a genetic disorder, a neuronal disorder, an eye disorder, a muscular disorder, or a cancer. 101. The method of any one of embodiments 97-100, wherein the protein is MeCP2, progranulin, dystrophin, or peripherin 2. 102. The method of any one of embodiments 97-101, wherein the protein is encoded by any one of the genes provided in TABLE 6. 103. The method of any one of embodiments 91-96, further comprising expressing a therapeutic polynucleotide encoded by the coding sequence in the target cell. 104. The method of embodiment 103, wherein a level of expression of the therapeutic polynucleotide is higher in the target cell than in the non-target cell. 105. The method of embodiment 103 or embodiment 104, wherein the therapeutic polynucleotide is a gRNA or a tRNA. 106. The method of any one of embodiments 103-105, wherein the therapeutic polynucleotide targets a gene associated with a genetic disorder, a neuronal disorder, an eye disorder, a muscular disorder, or a cancer. 107. The method of any one of embodiments 103-106, wherein the therapeutic polynucleotide targets a gene provided in TABLE 6. 108. A method of identifying a cell-specific promoter, the method comprising: introducing a promoter library to a population of target cells; wherein the promoter library comprises a plurality of candidate promoter sequences, wherein a candidate promoter sequence of the plurality of candidate promoter sequences comprises one or more transcription factor binding sequences and a core promoter sequence, and wherein the candidate promoter is linked to a unique barcode sequence; introducing the promoter library to a population of non-target cells; and identifying a cell-specific promoter as the candidate promoter sequence that promotes higher transcription of the unique barcode in the population of target cells than in the population of non-target cells. 109. The method of embodiment 108, wherein the population of target cells comprises a target cell type, and wherein the population of non-target cells comprises a non-target cell type. 110. The method of embodiment 109, wherein the cell-specific promoter is a cell-type specific promoter. 111. The method of embodiment 108, wherein the population of target cells comprises a target cell state, and wherein the population of non-target cells comprises a non-target cell state. 112. The method of embodiment 111, wherein the cell-specific promoter is a cell-state specific promoter. 113. The method of embodiment 108, wherein the population of target cells comprises diseased neurons comprising a MECP2 mutant gene and having a disease phenotype associated with protein expression from the MECP2 mutant gene, and wherein the population of non-target cells comprises healthy neurons comprising a wildtype MECP2 gene and having wild type phenotype associated with protein expression from the wild type MECP2 gene. 114. The method of embodiment 113, wherein the cell-specific promoter is specific for neurons expressing mutant MeCP2 protein. 115. The method of any one of embodiments 108-114, wherein the one or more transcription factor binding sequences are selected from endogenous transcription factor binding sequences, variant transcription factor binding sequences, engineered transcription factor binding sequences, and combinations thereof. 116. A recombinant core promoter comprising at least about 40%, at least about 50%, at least about 60%, at least about 70%, at least about 80%, at least about 90%, at least about 95%, or at least about 98% sequence identity to any one of SEQ ID NO: 8, SEQ ID NO: 10-SEQ ID NO: 22, SEQ ID NO: 25, or SEQ ID NO: 42. 117. The recombinant core promoter of embodiment 116 comprising any one of SEQ ID NO: 8, SEQ ID NO: 10-SEQ ID NO: 22, SEQ ID NO: 25, or SEQ ID NO: 42. 118. The recombinant core promoter of embodiment 116 or 117, consisting of any one of SEQ ID NO: 8, SEQ ID NO: 10-SEQ ID NO: 22, SEQ ID NO: 25, or SEQ ID NO: 42. 119. The recombinant core promoter of any of embodiments 116-118, wherein the recombinant core promoter is downstream of a transcription factor binding motif.

### EXAMPLES

The invention is further illustrated by the following non-limiting examples.

### Example 1

### Identification of Differentially Expressed Transcription Factors in a Rett Syndrome Model

This example describes identification of differentially expressed transcription factors in a Rett syndrome model, which were used to identify candidate transcription factors for MECP2 mutant cell-specific expression.

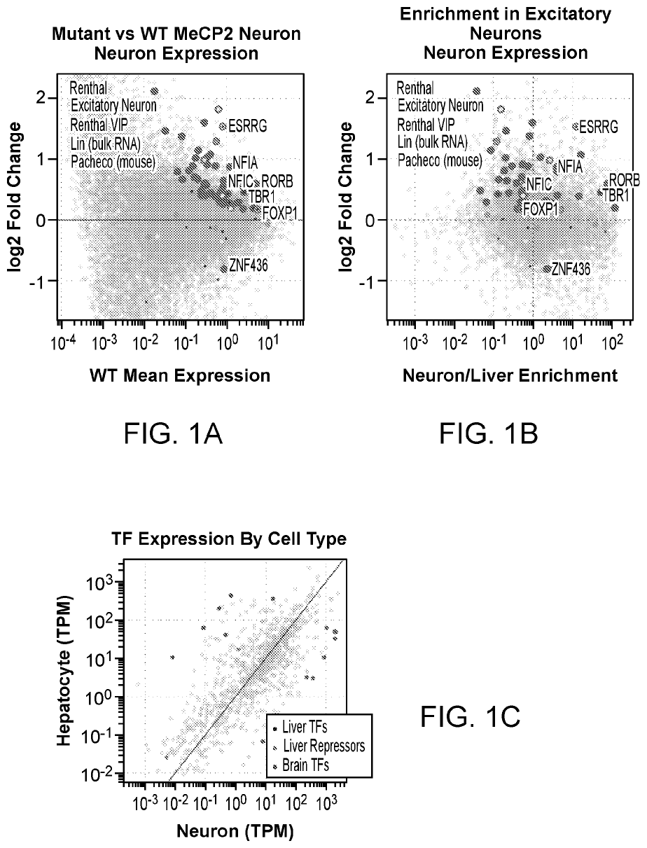

Transcription factor expression was compared in neurons expressing mutant MeCP2 protein and containing a mutation in the MECP2 gene (“mutant MeCP2 neurons”), a cause of Rett syndrome, and neurons expressing wild type MeCP2 protein and containing wild type MECP2 (“WT MeCP2 neurons”). FIG. 1A shows the fold-change in transcription factor expression in mutant MeCP2 neurons relative to WT MeCP2 neurons. Points in FIG. 1A corresponding to candidate transcription factors are shown as larger or darker grey points. Transcription levels of each transcription factor were determined using RNA-seq data from databases: the “Renthal Excitatory Neuron” and “Renthal VIP” are from excitatory neurons or VIP expressing neurons single-cell RNA sequencing data, respectively, from Renthalet al (Characterization of human mosaic Rett syndrome brain tissue by single-nucleus RNA sequencing. Nature neuroscience 21, 12 (2018): 1670-1679); “Lin (bulk RNA)” is from single-cell RNA sequencing data from Lin et al (Transcriptome analysis of human brain tissue identifies reduced expression of complement complex C1Q Genes in Rett syndrome. BMC Genomics 17, 427 (2016)); and “Pachecho (mouse)” is from single-cell RNA sequencing data from Pacheco et al (RNA sequencing and proteomics approaches reveal novel deficits in the cortex of Mecp2-deficient mice, a model for Rett syndrome. Molecular Autism 8, 56 (2017)), which are each incorporated by reference in their entirety. Candidate transcription factors points are shaded according to the database from which they were identified. FIG. 1B shows the fold-change in expression in excitatory neurons relative to hepatocytes. Points corresponding to candidate transcription factors shown as larger or darker grey points in FIG. 1A are also shown as larger or darker grey points in FIG. 1B. FIG. 1C shows transcription factor (TF) expression in hepatocytes, in transcripts per kilobase million (TPM), relative to neurons.

Eighty-nine candidate transcription factors were identified, representing transcription factors that are both expressed in neurons and are differentially expressed in neurons with mutant versus wild type MeCP2. The 89 candidate transcription factors and corresponding neuron to liver expression ratios are provided in TABLE 1. Of these 89 candidates, 51 were identified from single cell neuron data, 9 from human bulk RNA-seq data, 15 from mouse bulk RNA-seq data, 5 were liver specific, and 9 are brain specific.

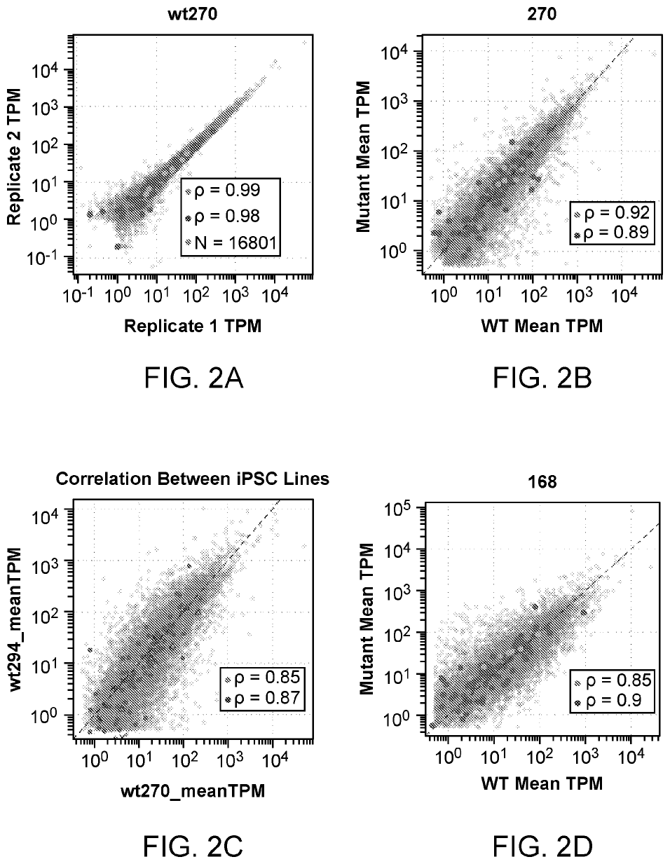

Transcription factor transcript levels were further analyzed in Rett patient induced pluripotent stem cell (iPSC) lines. FIG. 2A, FIG. 2B, FIG. 2C, and FIG. 2D show RNA-seq data of transcript levels for candidate transcription factors in transcripts per million (TPM). Points corresponding to all evaluated transcription factors are shown. Transcription factor transcript level of the 89 candidate MECP2 mutant cell-specific transcription factors are shown as darker grey points. The top ten transcription factor candidates of these are shown as lighter grey points. FIG. 2A shows transcript levels of transcription factors between two wild type MeCP2 neuronal cell replicates derived from a Rett patient iPSC line. The strong linear correlation between the repeats demonstrates that RNA-seq produced consistent results. FIG. 2B shows correlation of transcript levels of transcription factors between wild type MeCP2 and mutant MeCP2 neuronal cells derived from a Rett patient iPSC line. FIG. 2C shows correlation of transcript levels of transcription factors between a wild type MeCP2 neuronal cell derived from a first Rett patient iPSC line and a wild type MeCP2 neuronal cell derived from a second Rett patient iPSC line. FIG. 2D shows correlation of expression levels between wild type MeCP2 and mutant MeCP2 neurons in neuronal cells derived from a third Rett patient iPSC line.

### Example 2

### Engineering Promotors to Tune Payload Expression

This example describes engineering promoters to tune payload (e.g., transgene) expression. Promoters containing a core promoter and one or more transcription factor binding sites are engineered to alter expression levels of a payload under control of the promoter. Expression levels are tuned for a specific cell state of interest, such as a differentiated cell type, a cell morphology, a cell phenotype, or a cell genotype. FIG. 3 illustrates approaches to engineering promoters with an inducible core promoter scaffold and a transcription factor binding sequence. Potential transcription factor binding sequences can be identified based on cell state specific transcription factor expression levels, such as candidate transcription factors identified by RNA-seq. Promoter libraries can be introduced into neurons expressing wild type MeCP2 (“WT MeCP2 neuron”) or into neurons expressing mutant MeCP2 (“mutant MeCP2 neuron”). RNA transcripts in WT MeCP2 neurons, DNA in WT MeCP2 neurons, RNA transcripts in mutant MeCP2 neurons, and DNA in mutant MeCP2 neurons can be sequenced and quantified to determine a transcription ratio between the two cell types. Promoters showing increased transcription in mutant MeCP2 neurons relative to WT MeCP2 neurons may contain binding sites for one or more transcription factors with enhanced expression in diseased neurons (e.g., mutant MeCP2 neurons). Transcription levels of each promoter may be determined using RNA sequencing (RNA-seq). Binding motifs for candidate transcription factors identified in EXAMPLE 1 are inserted into the inducible core promoter scaffold. The scaffold contains a background sequence that separates the transcription factor binding sequences and the core promoter sequence. Background sequences are screened, and the selected background sequence does not introduce transcriptional noise and/or does not affect transcriptional activation. Single transcription factor binding motifs, such as a motif that binds to the ESRRG transcription factor, are inserted into the promoter. Promoters containing two, three, or four duplicates of the transcription factor are screened, along with different combinations of transcription factor binding motifs. Different combinations and duplications of transcription factor binding motifs may result in different levels of transgene expression. Binding sites for transcriptional repressors, such as ZNF436, are added to further tune expression levels. Introducing mutations in the core promoter may alter expression levels by affecting transcription initiation by RNA polymerase II. Payload (e.g., transgene) expression levels are cell state dependent.

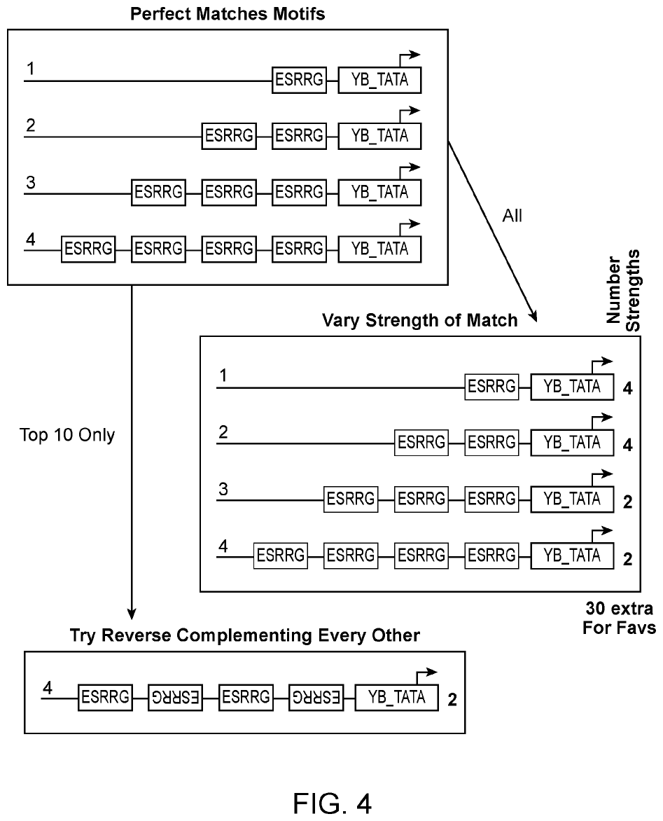

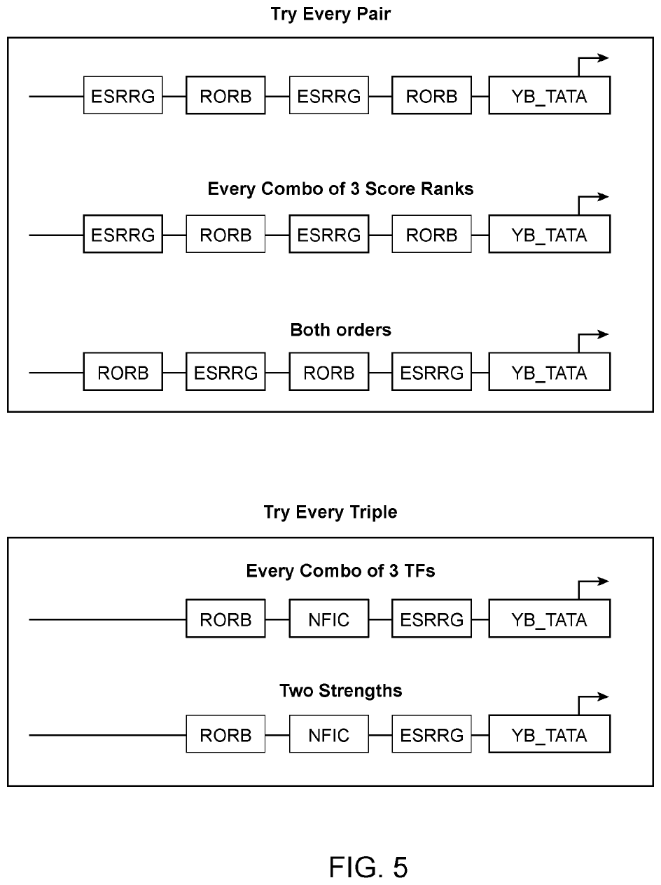

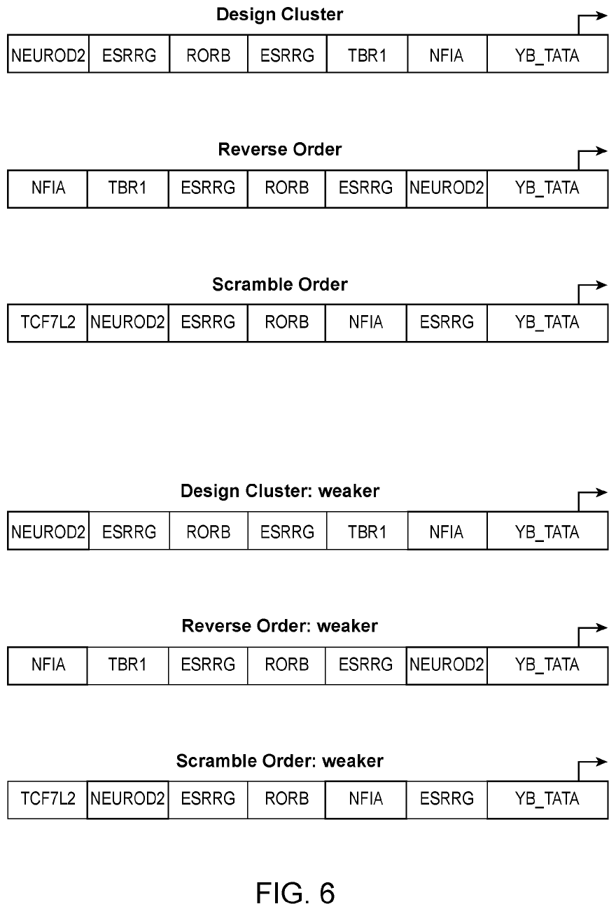

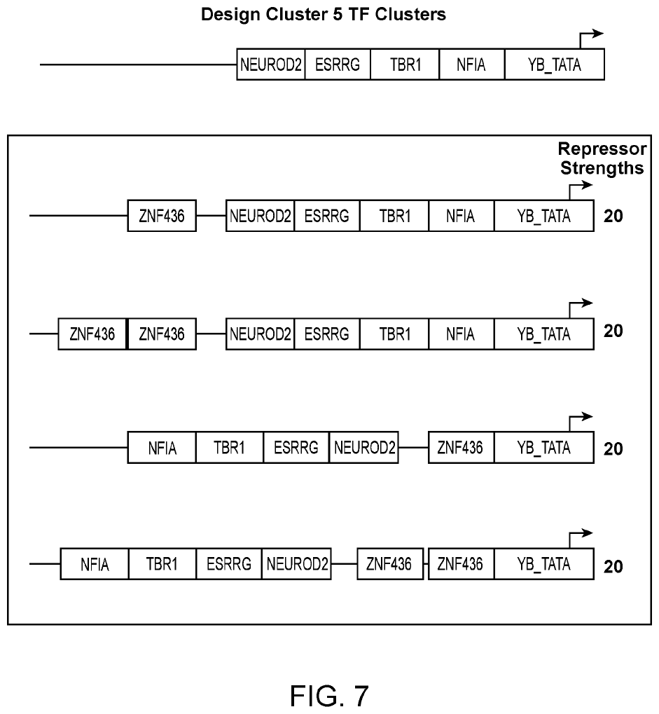

FIG. 4 illustrates a screening strategy to tune payload (e.g., transgene) expression. A screening strategy can be a massively parallel reporting assay (MPRA), e.g., using the different combinations of transcription factor binding motifs as disclosed in this EXAMPLE in a library of candidate promoters as described EXAMPLE 3. Transcription factor binding motifs with perfect sequence matches to the consensus transcription factor binding motif are screened with different numbers of duplications. Match strength is varied to alter the binding affinity of the motif for the transcription factor by mutating the motif sequence relative to the consensus sequence. Reverse complements of transcription factor binding motifs are also screened. As illustrated in FIG. 5, pair-wise and triple combinations of transcription factor binding motifs are screened. The order of the motifs is rearranged and scrambled for perfect match and varied strength motifs, as shown in FIG. 6. The effect of repressors on expression levels is tested, as shown in FIG. 7. Core promoter sequences that are screened to tune transgene expression levels are shown in FIG. 8.

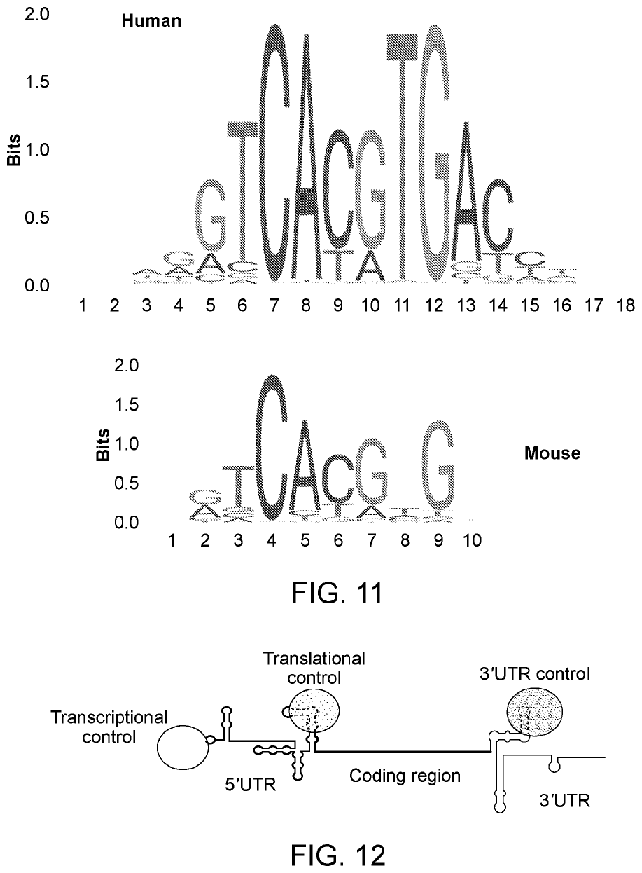

Match strength of a transcription factor binding motif is related to the sequence preference of a transcription factor. A transcription factor binding motif consensus sequence is determined by screening nucleotide sequences for transcription factor binding and identifying the most enriched nucleotides in each position. The consensus sequence represents a sequence preferred by the corresponding transcription factor. FIG. 9 illustrates nucleotide enrichment in motifs that bind to ESRRG, RORA, or RORB transcription factors. The largest nucleotide letters at each position represent the consensus motif. FIG. 10 illustrates nucleotide enrichment in motifs that bind to NFIA, NFIC, NFIC, or NFYC transcription factors. Preferred transcription factor binding motifs are species-dependent for some transcription factors. FIG. 11 illustrates sequence enrichment for ESRRG in human neurons and mouse neurons. ESRRG binds the same preferred motif in human and mouse neurons.

### Example 3

### Generation of Candidate Promoter Libraries for Massively Parallel Reporting Assay Screens

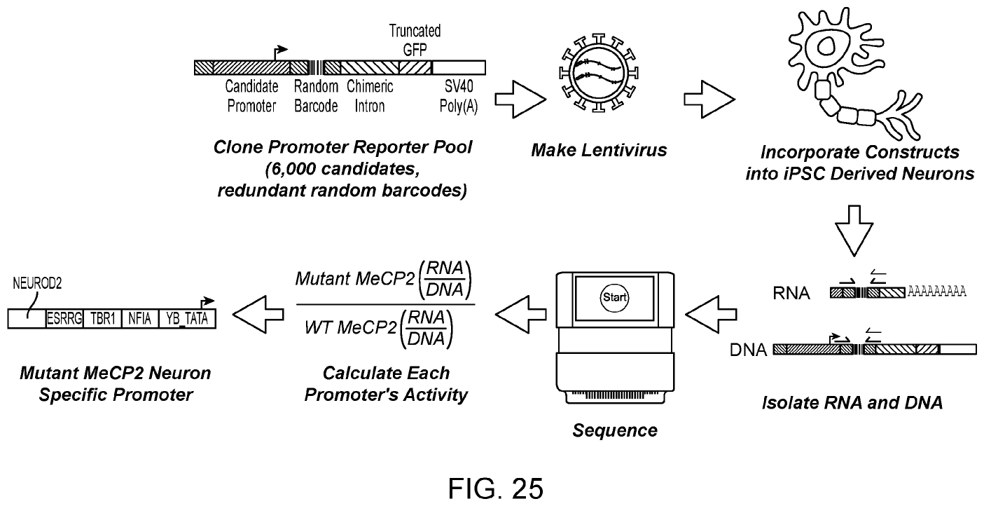

This example describes generation of candidate promoter libraries for use in a massively parallel reporting assay (MPRA) to identify cell type- or cell state-specific promoters. A workflow for performing an MPRA to identify cell type- or cell state-specific promoters is shown in FIG. 25. A library of candidate promoters is synthesized and redundant random barcodes are attached, which are then inserted into lentiviral vector reporter constructs. The candidate promoter library lentiviral reporter constructs are packaged into lentivirus and introduced into one or more cell populations having a particular cell type or cell state. Transcriptional activation is measured by sequencing barcoded RNA transcripts under control of the candidate promoters and determining the activity of each candidate promoter. To determine cell type- or cell state-specificity of a promoter, activity is compared across different cell populations having a different cell type or cell state.

### Example 4

### Identification of Cell Type- or Cell State-Specific Promoters Using a Massively Parallel Reporting Assay

This example describes identification of cell type- or cell state-specific promoters using a massively parallel reporting assay (MPRA). To identify mutant MeCP2-specific neuronal promoters, the MPRA was performed with the candidate promoters for the candidate transcription factors identified in EXAMPLE 1 in wild type MeCP2 neurons and in mutant MeCP2 neurons. Briefly, candidate promoter libraries were generated as described in EXAMPLE 3. The library included promoters containing transcription factor binding motifs identified from human and mouse date and promoters containing de novo designed transcription factor binding motifs. Motifs were combined in varying number and combination and combined with core promoters (e.g., core promoters from TABLE 4), for example, as illustrated in FIG. 3-FIG. 5. Promoter sequences were engineered to remove inverted motifs that may form hairpins that could interfere with sequence amplification. Candidate promoters were tested with two different background sequences.

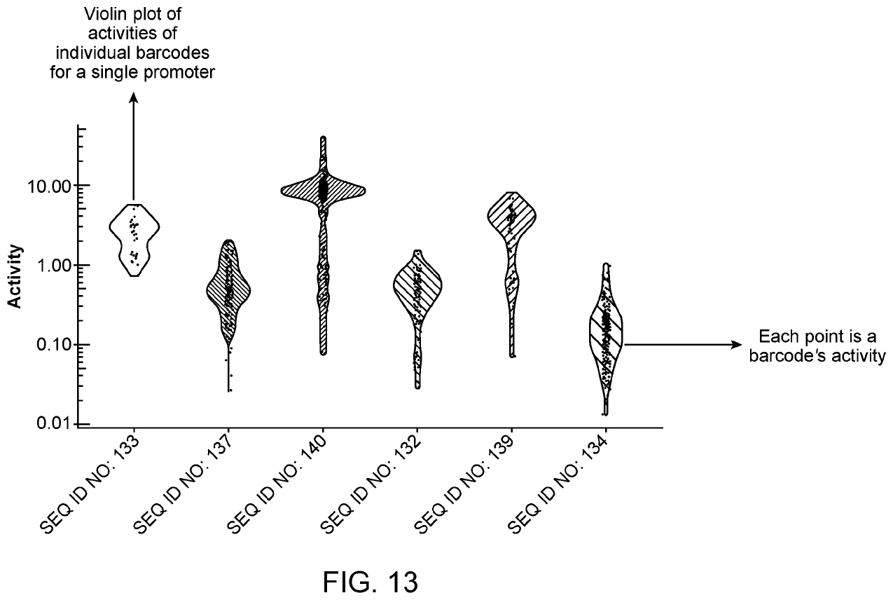

As illustrated in FIG. 25, transcription ratios from each candidate promoter were determined in wild type MeCP2 neurons relative to mutant MeCP2 neurons. Candidate promoters with higher transcriptional activity in mutant MeCP2 neurons compared to in wild type MeCP2 neurons were identified as mutant MeCP2-specific neuronal promoters. More specifically, transcription activation was measured in induced pluripotent stem cells having a mutation in MeCP2. Transcriptional activation was validated by comparing activation across all redundant barcodes for a selection of promoters. As shown in FIG. 13, similar transcriptional activity was seen for each promoter sequence across redundant barcodes, demonstrating that they assay provided reliable quantification of transcriptional activation.

### Example 5

### Effect of Motif Duplication and Pairing on Transcriptional Activation

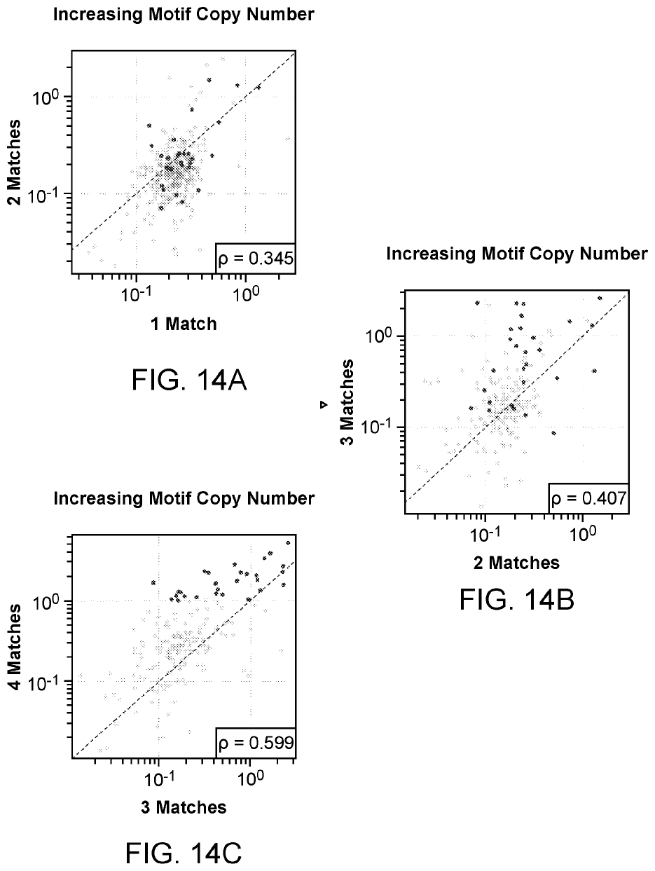

This example describes the effect of motif duplication and pairing on transcriptional activation of engineered promoter sequences. To test the effect of motif duplication on transcriptional activation, promoters containing one motif match (such as the “Single Motif Match” shown in FIG. 3), two motif matches (such as the “Two Matches” shown in FIG. 3), three motif matches (such as the “Three” shown in FIG. 3), or four motif matches (such as the “Four” shown in FIG. 3) were compared. Pairwise comparisons of the same transcription factor binding motif present in a promoter with either one copy or two copies is shown in FIG. 14A; pairwise comparisons of the same transcription factor binding motif present in a promoter with either two copies or three copies is shown in FIG. 14B; and pairwise comparisons of the same transcription factor binding motif present in a promoter with either three copies or four copies is shown in FIG. 14C. In each of FIG. 14A-FIG. 14C, the transcription factor binding motifs showing the highest activation when present at four copies are marked with darker grey dots. These data indicated that the differences in effects of transcription factor binding motifs on transcriptional activation were most prominent with the motif was present at four copies as compared to fewer copies.

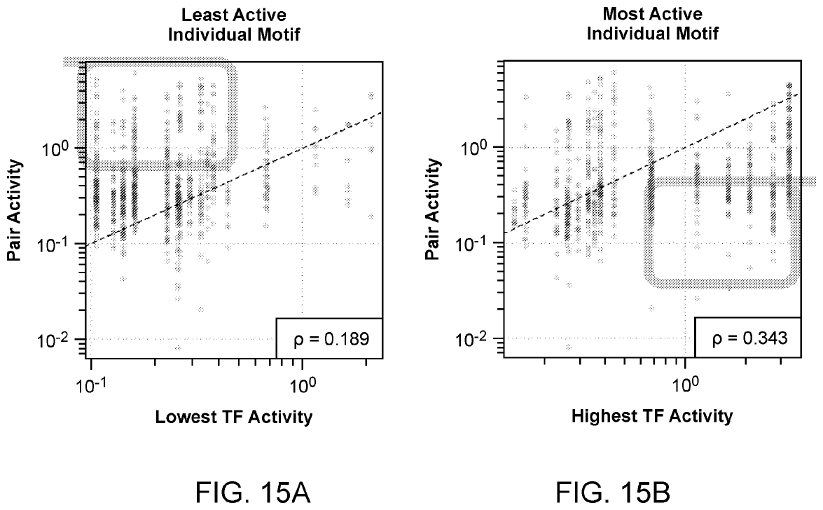

To test synergy of different motif pairs on transcriptional activation, promoters containing all possible combinations of duplicated pairs for the top the transcription factor binding motifs (such as the “Every Pair of Favorite 10” shown in FIG. 3) were tested. Transcriptional activation of each duplicated pair was plotted as a function of the activity of lowest activity transcription factor binding motif in each pair (FIG. 15A) or the highest activity transcription factor binding motif in each pair (FIG. 15B) when present as four of the same motif. In FIG. 15A, the box denotes synergistic transcription factor binding motif pairs that exhibited higher activity than the individual motifs. In FIG. 15B, the box denotes “lone wolf” transcription factor binding motifs that exhibited higher activity as individual motifs than when paired.

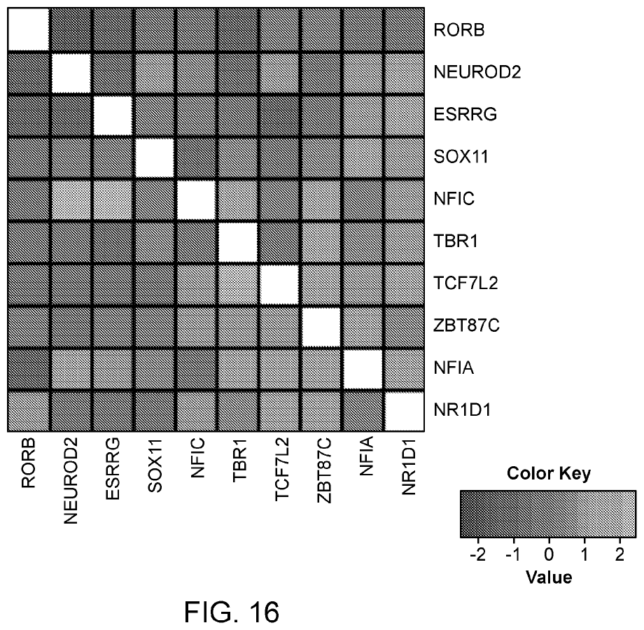

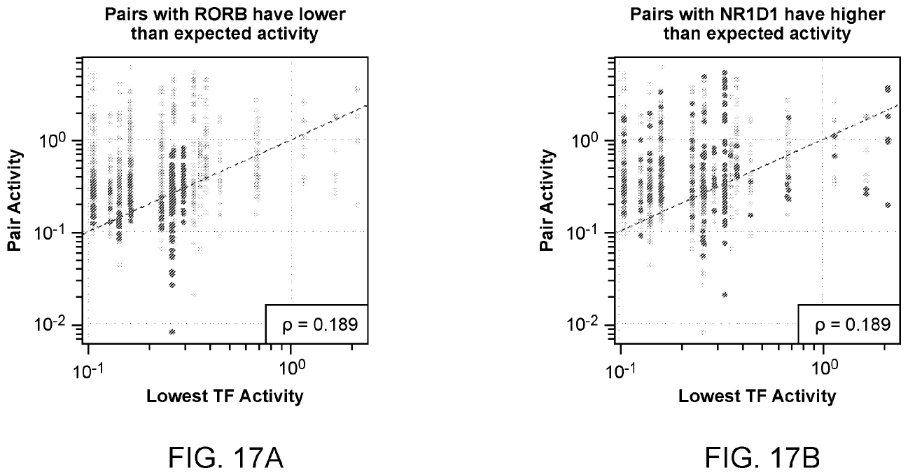

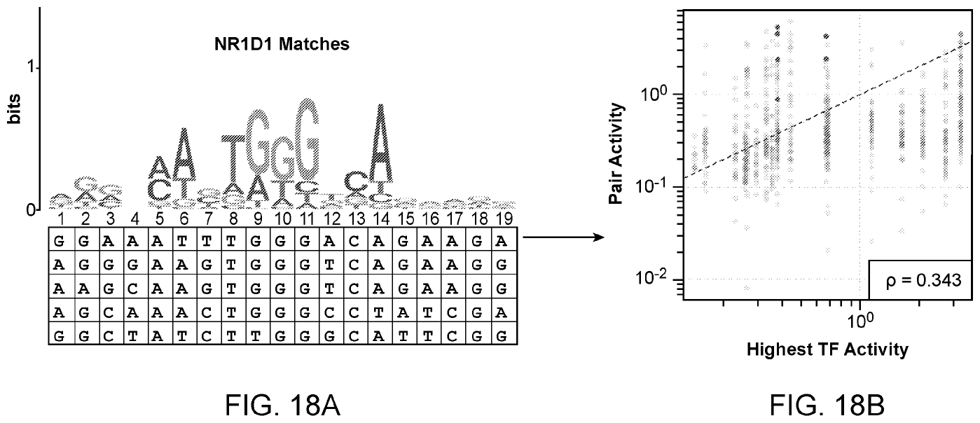

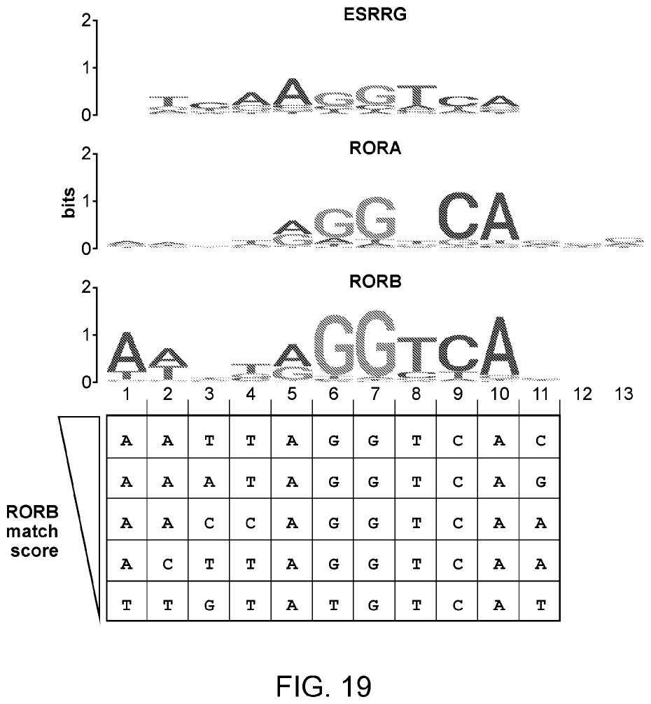

A heatmap showing transcriptional activation of specific transcription factor binding motif pairs is shown in FIG. 16. The first element of the pair, from 5′ to 3′, is shown on the x-axis, and the second element of the pair is shown on the y-axis. Promoters containing combinations of RORB motifs (e.g., SEQ ID NO: 87-SEQ ID NO: 92), NEUROD2 motifs (e.g., SEQ ID NO: 65, SEQ ID NO: 66, or SEQ ID NO: 110), ESRRG motifs (e.g., SEQ ID NO: 47, SEQ ID NO: 48, or SEQ ID NO: 112), SOX11 motifs (e.g., SEQ ID NO: 106), NFIC motifs (e.g., SEQ ID NO: 69, SEQ ID NO: 70, or SEQ ID NO: 104), TBR1 motifs (e.g., SEQ ID NO: 93 or SEQ ID NO: 94,), TCF7L2 motifs (e.g., SEQ ID NO: 95, SEQ ID NO: 96, SEQ ID NO: 105, or SEQ ID NO: 109), ZBTB7C motifs (e.g., SEQ ID NO: 97 or SEQ ID NO: 98), NFIA motifs (e.g., SEQ ID NO: 67 or SEQ ID NO: 68), and NR1D1 motifs (e.g., SEQ ID NO: 71-SEQ ID NO: 76) were compared. The lack of symmetry in the heatmap indicates that the order of the motifs affected activation. Of the motifs tested, RORB-binding motifs (e.g., SEQ ID NO: 87-SEQ ID NO: 92), exhibited lower transcriptional activation when paired than individually, as shown in FIG. 17A, which shows motif pairs containing the RORB-binding motif denoted with darker grey dots. Conversely, NR1D1-binding motifs (e.g., SEQ ID NO: 71-SEQ ID NO: 76), exhibited higher transcriptional activation when paired than individually, as shown in FIG. 17B, which shows motif pairs containing an NR1D1-binding motif denoted with darker grey dots. Together these data indicate that certain transcription factor binding motifs exhibit higher activity individually than in pairs, and that other transcription factor binding motifs exhibit higher activity when paired with a different motif than individually. One NR1D1-binding motif (SEQ ID NO: 72), shown in FIG. 18A along with other NR1D1-binding motifs, was present in many of the highly synergistic pairs, marked with darker grey dots in FIG. 18B. Additional RORB-binding motifs are shown in FIG. 19, along with the relative match score for RORB-binding.

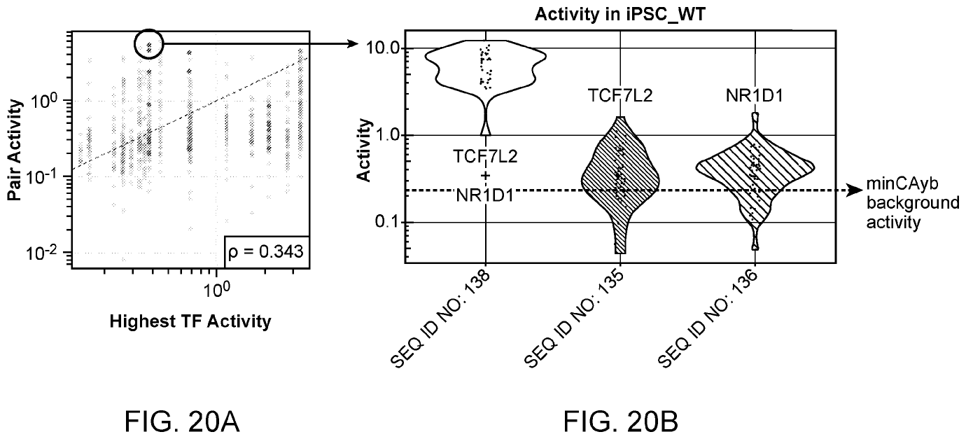

One synergistic motif containing a duplicated TCF7L2-binding motif and NR1D1-binding motif pair, indicated with a circle in FIG. 20A, was selected for further analysis. A promoter containing the motif pair (SEQ ID NO: 138) exhibited about 50-fold higher transcriptional activity in wild type iPSCs than a promoter containing four TCF7L2-binding motifs (SEQ ID NO: 135) or a promoter containing four NR1D1-binding motifs (SEQ ID NO: 136), as shown in FIG. 20B.

### Example 6

### Screening Rationally and De Novo Designed Promoters for Mutant MeCP2-Specific Activity

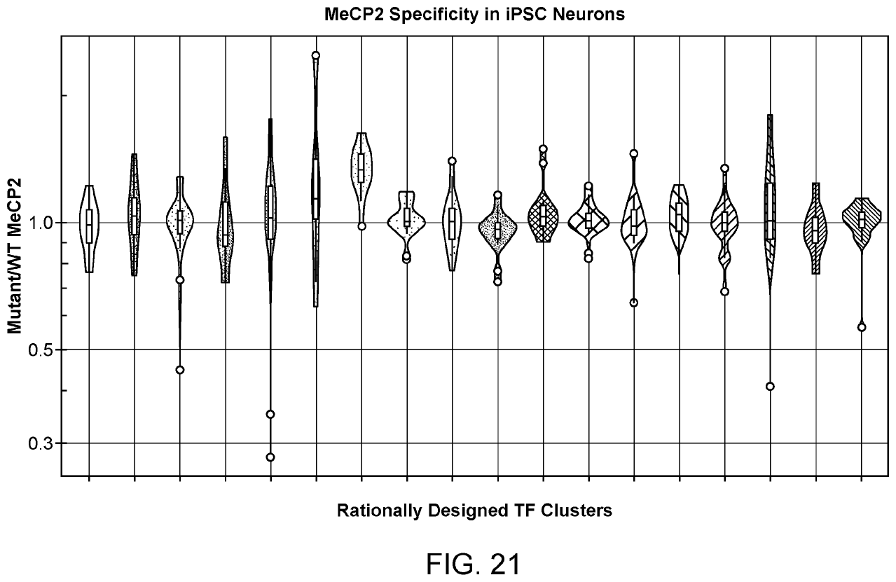

This example describes screening for rationally and de novo designed promoters that exhibit mutant MeCP2-specific transcriptional activation. Candidate transcription factor binding sequences were either rationally designed or designed de novo. Rationally designed candidate transcription factor binding sequences were designed based on the transcription factor binding motif screens described in EXAMPLE 11. Candidate sequences were screened based on their ability to promote differential transcriptional activation in neurons derived from iSPCs with either mutant or wild type MeCP2, as shown in FIG. 21. FIG. 21 shows transcriptional activation of, from left to right, SEQ ID NO: 39, SEQ ID NO: 31, SEQ ID NO: 36, SEQ ID NO: 29, SEQ ID NO: 30, SEQ ID NO: 28, SEQ ID NO: 26, SEQ ID NO: 38, SEQ ID NO: 33, SEQ ID NO: 27, SEQ ID NO: 44 (AGAAGAACAACCGTACGCCACTAACGATCGAAGCTTGATCAATTGAAGAATAATA GTGGACCAGCCGGTATCCACAGTCTCAAGAGAGAGGACAGGCCGGTATCGACTCAA GCGACAGGACCTACTTAATTGAGGTAATATTCGTTGTCGAGTAGAATTATTCCTATA CC), SEQ ID NO: 141 (AAGGTAGCTTCCAGTACGCCTCGTTACTTCGGAGTTACGTATACTCACGCGTAAGT TGCCGAATAGGTGCACTATGACTGGAGTGCTTAGCGCGTGATTACTGCTGGAGGAT TGGAATTGGCGATTCTTACGCGGAACCACGATAACGAGATAACGTTAAGTCGCTAG AC), SEQ ID NO: 32, SEQ ID NO: 35, SEQ ID NO: 41, SEQ ID NO: 34, SEQ ID NO: 40, and SEQ ID NO: 37. Promising candidates, such as SEQ ID NO: 26-SEQ ID NO: 41, exhibited higher transcriptional activation in cells with mutated MeCP2 than in cells with wild type MeCP2.

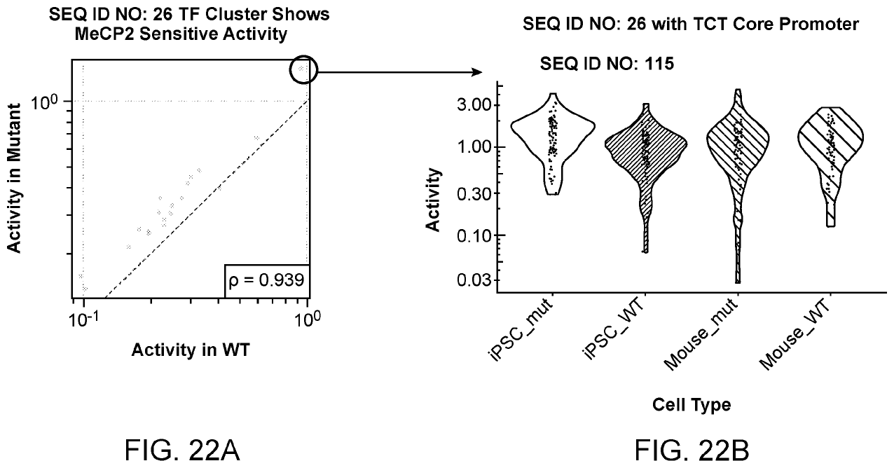

De novo sequences were designed based on sequence motifs that were enriched near promoters in genes differentially expressed in patient cells. The de novo sequences were screened for higher transcriptional activity in cells expressing mutated MeCP2 than in cells expressing wild type MeCP2, as shown in FIG. 22A. Promoters with selective activity in cells expressing mutated MeCP2 appear above the dotted line corresponding to y=x in FIG. 22A. One candidate promoter, SEQ ID NO: 115 containing a de novo transcription factor binding sequence of SEQ ID NO: 26 and a core promoter of SEQ ID NO: 9, showed high selectivity for cells expressing mutated MeCP2. The activity of SEQ ID NO: 26 in cells expressing mutated MeCP2 (“iPSC_mut”), cells expressing wild type MeCP2 (“iPSC_WT”), mouse cells expressing mutated MeCP2 (“mouse_mut”), or mouse cells expressing wild type MeCP2 (“mouse_WT”) is shown in FIG. 22B.

### Example 7

Identification of Promoters with Mutant MeCP2-Specific Activity

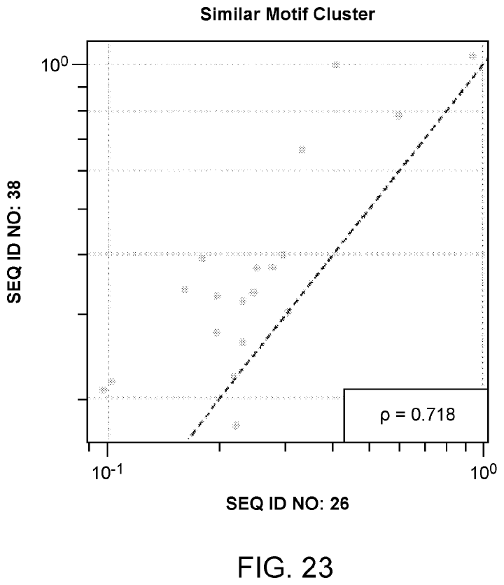

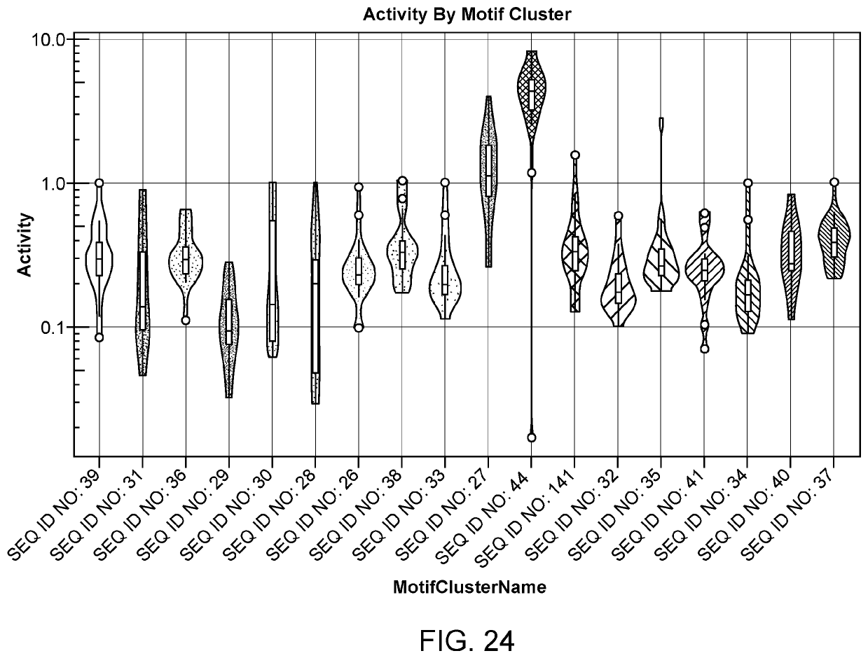

This example describes identification of promoters that exhibit mutant MeCP2-specific transcriptional activation. Twenty core promoter sequences were tested in combination with 18 rationally designed transcription factor binding sequences (SEQ ID NO: 27-SEQ ID NO: 37 and SEQ ID NO: 39-SEQ ID NO: 41) and two de novo designed transcription factor binding sequences (SEQ ID NO: 26 and SEQ ID NO: 38) for mutant MeCP2-specific transcriptional activation. Each combination of a core promoter and a transcription factor binding sequence was tested. As shown in FIG. 23, the two de novo designed transcription factor binding motifs SEQ ID NO: 26 and SEQ ID NO: 38) exhibited activity that was well correlated across all core promoter combinations. The promoter sequence of 115 containing a core promoter of 9 and a de novo designed transcription factor binding motif of SEQ ID NO: 26 was identified as a candidate for specific transcriptional activity in mutant MeCP2 neurons. Activity transcription factor binding sequence with various core promoters is shown in FIG. 24.

### Example 8

### Cell State Specific Transgene Delivery to a Subject

This example describes cell state specific transgene delivery to a subject. A viral vector containing a polynucleotide encoding a viral inverted terminal repeat sequence, a promoter sequence, and a transgene sequence encapsulated in a viral capsid is generated. The transgene sequence encodes a protein to be expressed in a target cell state. The target cell state is a disease phenotype, disease genotype, and/or a cell type. The promoter sequence comprises a core promoter sequence and one or more transcription factor binding motifs and is engineered to promote transcription of the transgene in a cell state specific manner, as described in EXAMPLE 1, EXAMPLE 2, and EXAMPLE 3. The promoter sequence is engineered to promote increased transcription of the transgene in the target cell state relative to cells not in the target cell state. Additionally, the promoter sequence is engineered to tune the transcription level of the transgene to a desired level, preventing over-expression or under-expression of the protein encoded by the transgene.

The viral vector is administered to a subject. Cell state specific transcription of the transgene results in increased transcription levels in cells in the target cell state relative to cells not in the target cell state. The protein encoded by the transgene is expressed in cells in the target cell state at the desired level. Tuning the protein expression level in the target cell state reduces adverse effects in the subject relative to systemic expression of the protein.

### Example 9

### Treating Rett Syndrome in a Subject Using Cell State Specific Transgene Delivery

This example describes treating Rett syndrome in a subject using cell state specific transgene delivery. Healthy neurons (e.g., neurons expressing wild-type MeCP2) and diseased neurons (e.g., neurons expressing mutant MeCP2) from a subject with Rett syndrome are screened for phenotype-specific transcription factor expression. Briefly, neuronal cell lines are generated from induced pluripotent stem cells collected from the subject with either wild type MeCP2 protein expression from a wild type MECP2 gene expression or mutant MeCP2 protein expression from a mutated MECP2 gene expression. A library of engineered promoters is screened for differential transcription in the mutant versus wild type MeCP2 neurons. Additionally, the promoter library is screened for desired transcription levels in the mutant MeCP2 neurons. Transcription levels are determined using RNA-seq. A promoter that selectively promotes transcription in mutant MeCP2 neurons at desired levels is selected.

A viral vector containing a polynucleotide encoding a viral inverted terminal repeat sequence, the selected promoter sequence, and a wild type MECP2 sequence encapsulated in a viral capsid is generated. The promoter sequence contains a core promoter sequence and one or more transcription factor binding motifs. The viral vector is administered to the subject. Upon administration, the MECP2 sequence from the viral vector is selectively transcribed in neurons having a disease phenotype associated with expression of mutant MeCP2, resulting in cell state specific expression of MeCP2 protein in diseased neurons. Expression levels of the exogenous MeCP2 protein (e.g., the MeCP2 expressed from the transgene) are tuned to prevent adverse effects due to over-expression of MeCP2, such as seizures, or under-expression of MeCP2, such as neurological impairment. Cell state specific expression of the exogenous MeCP2 protein reduces one or more symptoms of Rett syndrome, thereby treating the Rett syndrome in the subject.

### Example 10

### Treating Frontotemporal Dementia in a Subject Using Cell Type Specific Transgene Delivery

This example describes treating frontotemporal dementia (FTD) in a subject using cell type specific transgene delivery. A viral vector containing a polynucleotide encoding a viral inverted terminal repeat sequence, a neuron-specific promoter sequence, and a wild type progranulin sequence encapsulated in a viral capsid is generated. The promoter sequence contains a core promoter sequence and one or more transcription factor binding motifs that bind to neuron-specific transcription factors. The viral vector is administered to the subject. Upon administration, the progranulin sequence from the viral vector is selectively transcribed in neurons, resulting in cell type specific expression of exogenous progranulin protein in neurons. Cell type specific expression of the exogenous progranulin reduces one or more symptoms associated with, prevents, or slows the progression of the frontotemporal dementia, thereby treating the frontotemporal dementia in the subject.

### Example 11

### Treating Cancer in a Subject Using Cell State Specific Transgene Delivery

This example describes treating cancer in a subject using cell state specific transgene delivery. Healthy and cancerous cells from a subject with cancer are screened for genotype-specific transcription factor expression. Briefly, healthy and cancerous cell lines are generated from the cells collected from the subject. A library of engineered promoters is screened for differential transcription in the healthy versus cancerous cells. Transcription levels are determined using RNA-seq. A promoter that selectively promotes transcription in the cancer cells is selected.

A viral vector containing a polynucleotide encoding a viral inverted terminal repeat sequence, the selected promoter sequence, and a pro-apoptotic sequence encapsulated in a viral capsid is generated. The promoter sequence contains a core promoter sequence and one or more transcription factor binding motifs. The viral vector is administered to the subject. Upon administration, the pro-apoptotic sequence from the viral vector is selectively transcribed in cancer cells, resulting in cell state specific expression of a pro-apoptotic protein in cancer cells. Expression of the pro-apoptotic protein in the cancer cells induces apoptosis of the cancer cells. Cell state specific expression of the pro-apoptotic protein kills or slows the progression of the cancer cells, thereby treating the cancer in the subject.

### Example 12

### Treating Macular Degeneration in a Subject Using Cell Type Specific Transgene Delivery

This example describes treating macular degeneration in a subject using cell type specific transgene delivery. A viral vector containing a polynucleotide encoding a viral inverted terminal repeat sequence, a retinal-specific promoter sequence, and a wild type PRPH2 sequence encapsulated in a viral capsid is generated. The promoter sequence contains a core promoter sequence and one or more transcription factor binding motifs that bind to retinal-specific transcription factors. The viral vector is administered to the subject. Upon administration, the PRPH2 sequence from the viral vector is selectively transcribed in retinal cells, resulting in cell type specific expression of exogenous peripherin 2 protein in retinal cells. Cell type specific expression of the exogenous peripherin 2 reduces symptoms associated with, prevents, or slows the progression of the macular degeneration, thereby treating the macular degeneration in the subject.

### Example 13

### Treating Duchenne's Muscular Dystrophy in a Subject Using Cell Type Specific Transgene Delivery

This example describes treating Duchenne's muscular dystrophy in a subject using cell type specific transgene delivery. A viral vector containing a polynucleotide encoding a viral inverted terminal repeat sequence, a muscle-specific promoter sequence, and a wild type DMD sequence encapsulated in a viral capsid is generated. The promoter sequence contains a core promoter sequence and one or more transcription factor binding motifs that bind to muscle-specific transcription factors. The viral vector is administered to the subject. Upon administration, the DMD sequence from the viral vector is selectively transcribed in muscle cells, resulting in cell type specific expression of exogenous dystrophin protein in muscle cells. Cell type specific expression of the exogenous dystrophin reduces symptoms associated with, prevents, or slows the progression of Duchenne's muscular dystrophy, thereby treating the Duchenne's muscular dystrophy in the subject.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A recombinant transcription factor binding polynucleotide comprising a sequence having at least 95% sequence identity to SEQ ID NO: 26.

2. The recombinant transcription factor binding polynucleotide of claim 1, comprising the sequence of SEQ ID NO: 26.

3. The recombinant transcription factor binding polynucleotide of claim 1 or claim 2, wherein the recombinant transcription factor binding polynucleotide is capable of binding to a transcription factor, optionally, wherein the transcription factor is expressed more highly in a target cell than in a non-target cell.

4. The recombinant transcription factor binding polynucleotide of claim 3, wherein the target cell is a cell expressing a mutant protein, and wherein the non-target cell is a cell expressing a wild type protein.

5. The recombinant transcription factor binding polynucleotide of claim 3 or claim 4, wherein the target cell expresses a mutant MeCP2 protein, and wherein the non-target cell expresses a wild type MeCP2 protein.

6. The recombinant transcription factor binding polynucleotide of any one of claims 1-5 comprising DNA; optionally, wherein the recombinant transcription factor binding polynucleotide consists of DNA.

7. A recombinant polynucleotide comprising a promoter and a payload,
wherein the promoter comprises:
a transcription factor binding polynucleotide capable of binding to a transcription factor, wherein the transcription factor binding polynucleotide comprises the recombinant transcription factor binding polynucleotide of any one of claims 1-6, and
a core promoter capable of recruiting a polymerase;
wherein the payload comprises a coding sequence.

8. The recombinant polynucleotide of claim 7, wherein the promoter comprises:
a) a sequence having at least 90% sequence identity to any one of SEQ ID NO: 113-SEQ ID NO: 131;
b) a sequence having at least 95% sequence identity to any one of SEQ ID NO: 113-SEQ ID NO: 131;
c) a sequence of any one of SEQ ID NO: 113-SEQ ID NO: 131;
d) a sequence having at least 90% sequence identity to SEQ ID NO: 115;
e) a sequence having at least 95% sequence identity to SEQ ID NO: 115; or
f) a sequence of SEQ ID NO: 115.

9. The recombinant polynucleotide of claim 7 or claim 8, wherein the core promoter comprises a TATA box, an initiator sequence, an RNA polymerase binding sequence, a B recognition element, a CCAAT box, a Pribnow box, a sequence provided in TABLE 4, or combinations thereof.

10. The recombinant polynucleotide of any one of claims 7-9, wherein the coding sequence is capable of being transcribed by the polymerase upon binding of the transcription factor to the transcription factor binding polynucleotide and recruitment of the polymerase to the core promoter; optionally, wherein the polymerase is an RNA polymerase II.

11. The recombinant polynucleotide of any one of claims 7-10, wherein the coding sequence encodes a protein.

12. The recombinant polynucleotide of claim 11, wherein the protein is a neuronal protein; optionally, wherein the protein is associated with a genetic disorder, a neuronal disorder, or both.

13. The recombinant polynucleotide of any one of claims 7-12, wherein the protein is MeCP2.

14. The recombinant polynucleotide of any one of claims 7-10, wherein the coding sequence encodes a therapeutic polynucleotide; optionally, wherein the therapeutic polynucleotide is a gRNA or a tRNA.

15. The recombinant polynucleotide of claim 14, wherein the therapeutic polynucleotide targets a gene associated with a genetic disorder, a neuronal disorder, or both.

16. The recombinant polynucleotide of claim 14 or claim 15, wherein the therapeutic polynucleotide targets MECP2.

17. The recombinant polynucleotide of any one of claims 7-16, wherein the promoter is engineered to control a transcription level of the payload.

18. The recombinant polynucleotide of claim 17, wherein the transcriptional level is cell state-specific, cell type-specific, cell genotype-specific, or any combination thereof.

19. The recombinant polynucleotide of claim 17 or claim 18, wherein a transcriptional level in a target cell is at least 1.3-fold a transcriptional level in a non-target cell.

20. An engineered viral vector comprising the recombinant polynucleotide of any one of claims 7-19 in a viral vector; optionally, wherein the viral vector is an adenoviral vector, an adeno-associated viral vector, or a lentivector; optionally, wherein the recombinant polynucleotide comprises DNA.

21. The engineered viral vector of claim 20, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68.

22. The engineered viral vector of claim 20 or claim 21, wherein a viral capsid of the viral vector is from a first viral vector and a viral inverted terminal repeat sequence of the viral vector is from a second viral vector; optionally, wherein the first viral vector, the second viral vector, or both is an adeno-associated viral vector.

23. A pharmaceutical composition comprising the recombinant polynucleotide of any one of claims 7-19 or the engineered viral vector of any one of claims 20-22, and a pharmaceutically acceptable carrier.

24. A composition comprising the recombinant polynucleotide of any one of claims 7-19, the engineered viral vector of any one of claims 20-22, or the pharmaceutical composition of claim 23 for use in a method of treating a disorder in a subject in need thereof, the method comprising administering to the subject the composition, thereby treating the disorder.

25. The composition of claim 24, wherein a level of transcription of the coding sequence is higher in the target cell than in a non-target cell of the subject.

26. The composition of claim 24 or claim 25, wherein the disorder is a genetic disorder, a neuronal disorder, or both; optionally, wherein the disorder is Rett syndrome.

27. A composition comprising the recombinant polynucleotide of any one of claims 7-19, the engineered viral vector of any one of claims 20-22, or the pharmaceutical composition of claim 23 for use in a method of expressing a coding sequence in a target cell, the method comprising administering to the subject the composition, thereby expressing the coding sequence in the target cell.

28. The composition of any one of claims 25-27, wherein the transcription factor is present at a higher level in the target cell than in the non-target cell; optionally, wherein the transcription factor is more active in the target cell than in the non-target cell.

29. The composition of any one of claims 25-28, wherein the non-target cell is a healthy cell.

30. The composition of any one of claims 25-29, wherein the target cell is a neuron.

31. The composition of any one of claims 25-30, wherein the target cell is a diseased cell; optionally, wherein the diseased cell comprises a genetic mutation associated with the disorder and has a disease phenotype associated with the genetic mutation.

32. The composition of claim 31, wherein the diseased cell comprises a mutation in MECP2 and expresses a mutant MeCP2 protein.

33. The composition of any one of claims 25-32, wherein a level of transcription of the coding sequence is higher in the target cell than in a non-target cell; optionally, wherein the target cell is a mutant MeCP2 cell, and the non-target cell is a wild type MeCP2 cell.

34. The composition of any one of claims 25-33, wherein the method further comprises expressing a protein encoded by the coding sequence in the target cell; optionally, wherein a level of expression of the protein is higher in the target cell than in the non-target cell.

35. The composition of claim 34, wherein the protein is a neuronal protein.

36. The composition of claim 34 or claim 35, wherein the protein is associated with a genetic disorder, a neuronal disorder, or both; optionally, wherein the protein is MeCP2.

37. The composition of any one of claims 25-33, wherein the method further comprises expressing a therapeutic polynucleotide encoded by the coding sequence in the target cell; optionally, wherein the therapeutic polynucleotide is a gRNA or a tRNA.

38. The composition of claim 37, wherein a level of expression of the therapeutic polynucleotide is higher in the target cell than in the non-target cell.

39. The composition of claim 37 or claim 38, wherein the therapeutic polynucleotide targets a gene associated with a genetic disorder, a neuronal disorder, or both; optionally wherein the therapeutic polynucleotide targets MECP2.

40. The composition of any one of claims 37-39, wherein the therapeutic polynucleotide targets MECP2.

41. The composition of any one of claims 24-40, wherein the coding sequence is transcribed upon binding of the transcription factor to the transcription factor binding site and recruitment of the polymerase to the core promoter sequence.
